(19) United States
(12) Patent Application Publication
(10) Pub. No.: US 2011/0195090 A1
(43) Pub. Date: Aug. 11, 2011

(54) METHOD OF MAKING A VACCINE

(75) Inventor: Dimiter S. Dimitrov, Frederick (MD)

(73) Assignees: THE UNITED STATES OF AMERICA, AS REPRESENTED BY THE SECRETARY, DEPARTMENT OF HEALTH AND HUMAN; SERVICES, Rockville, MD (US)

(21) Appl. No.: 13/123,659

(22) PCT Filed: Oct. 11, 2009
(86) PCT No.: PCT/US2009/060303

§ 371 Date: Apr. 11, 2011

Related U.S. Application Data
(60) Provisional application No. 61/104,706, filed on Oct. 11, 2008.

(51) Int. Cl. A61K 39/21; A61K 39/00; A61P 35/00; A61P 31/18

(52) U.S. Cl. 424/208.1; 424/277.1; 424/184.1

(57) ABSTRACT

The present invention provides a vaccine and method for making same which is effective to elicit a desired antibody against a target antigen comprising a primary immunogen and a secondary immunogen, wherein the primary immunogen is effective to elicit B cell receptors (BCRs) that are on the maturational pathway of the desired antibody and have an intermediate degree of somatic mutational diversity, and the secondary immunogen comprises an epitope of the desired target antibody and is effective to further diversify the BCRs sufficient to form mature BCRs having the identical or substantially identical sequence as the desired antibody.

### INCORPORATION BY REFERENCE

This application claims priority from U.S. Provisional Application Ser. No. 61/104,706 filed Oct. 11, 2008 which is incorporated herein by reference in its entirety. Any and all references cited in the text of this patent application, including any U.S. or foreign patents or published patent applications, International patent applications, as well as, any non-patent literature references, including any manufacturer's instructions, are hereby expressly incorporated by reference.

### BACKGROUND OF THE INVENTION

1. Field of the Invention

This invention relates generally to methods of preparing vaccines, and to the use of such vaccines in the vaccination and treatment of human disease, e.g., immunodeficiency virus (HIV) infections and cancer.

2. Background

The development of a vaccine against human immunodeficiency virus (HIV) remains an unachieved goal more than two decades after its discovery. Vaccine development has been elusive and made difficult due to the fact that the virus rapidly mutates and “hides” conserved epitopes of its envelope glycoprotein by using variable loops, heavy glycosylation, oligomerization and conformational masking.

Enveloped viruses, such as HIV, enter cells by a two-step process. The first step involves the binding of a viral surface protein to receptors on the plasma membrane of a host cell. After receptor binding, a membrane fusion reaction takes place between the lipid bilayer of the viral envelope and host cell membranes. Viral proteins embedded in the lipid bilayer of the viral envelope catalyze receptor binding and membrane fusion reactions.

In HIV, the envelope (Env) glycoprotein performs the functions of viral entry. Env is synthesized as a polyprotein precursor molecule which is proteolytically processed by a host protease to generate the surface (gp120) and transmembrane subunits (gp41) of the mature Env glycoprotein complex. The unprocessed Env precursor is known as gp160, reflecting is apparent molecular mass, which is further processed to form the gp41 subunit and the gp120 subunit.

The initial step in HIV infection involves the binding of gp120 to the cell surface molecule CD4, which serves as the major receptor for HIV-1 and HIV-2. The membrane fusion process is initiated by the interaction of gp120 with a G protein-coupled co-receptor, either the CCR5 or the CXCR4 chemokine receptor, generally after prior contact of gp120 with CD4. Gp41 is involved in the fusion process. The exact role of gp41 in membrane fusion is not fully understood. In one theory, gp41 first engages contact with the target cell membrane by its amino-terminal hydrophobic domain, termed the fusion peptide, and then undergoes conformation changes in order to bring the viral and cellular lipid bilayers in proximity, allowing their external leaflets to merge, thereby forming a hemifusion intermediate. Next, an aqueous connection, termed a fusion pore, must open across the internal leaflets of the merged membranes and expand to leave open passage to the nucleocapsid.

An important goal in the quest for identifying an effective HIV vaccine has been the search for a vaccine immunogen that is capable of eliciting broadly cross-reactive HIV neutralizing antibodies (bcrnAbs) (equivalently as broadly neutralizing antibodies (bnAbs)). Such antibodies are rarely elicited in HIV-infected humans, and only several such monoclonal bcrnAbs are known, which include IgG b12 (Burton et al., 1994; Roben et al., 1994), IgG 2G12 (Trkola et al., 1996; Sanders et al., 2002; Scanlan et al., 2002), m14 (Zhang et al., 2004c), m18 (Zhang et al., 2003), 447-52D (Gorny et al., 1992), IgG 2F5 (Muster et al., 1993), IgG 4E10 (Stiegler et al., 2001; Zwick et al., 2001), IgG m46 (Choudhry et al., 2007), IgG m48 (Zhang et al., 2006), Fab X5 (Moulard et al., 2002) and Fab Z13 (Zwick et al., 2001), each of which are incorporated herein by reference in their entireties.

The existence of these human monoclonal antibodies has fueled the hope that it is possible to develop an effective appropriate vaccine immunogen containing the epitopes recognized by these bcrnAbs. However, in spite of the tremendous amount of research and money spent on this approach, the development of an HIV vaccine has thus far failed. The continued failures in the identification of a suitable immunogen capable of eliciting potent bcrnAbs in humans strongly suggests that there are still unknown fundamental immunological mechanisms that allow HIV to evade elicitation of such antibodies. Understanding these mechanisms could provide novel tools for development of efficacious vaccines.

Given the continued lack of an effective HIV vaccine despite the enormous costs and efforts expended since the first discovery of HIV/AIDS, the development of novel approaches for the identification of broadly cross-reactive neutralizing anti-HIV antibodies and HIV immunogens which are capable of eliciting such bcrnAbs are in dire need. Such methods advantageously should not be limited to the development of anti-HIV vaccines, but also should be applicable to the development of similarly effective vaccines against other human diseases, such as other viruses, cancer, and infectious micoorganisms, including bacteria, yeast, and protists.

### SUMMARY OF THE INVENTION

The present invention provides a new method for obtaining novel vaccines that are capable of eliciting antibodies, e.g., broadly cross reactive neutralizing antibodies (bcrnAbs) (or equivalently broadly neutralizing antibodies (bnAbs), which can be used as vaccines to combat a variety of human diseases and infectious agents which overcomes the various problems in the art. In a particular aspect, the present invention provides a new method for the development and preparation of anti-HIV vaccines which are capable of eliciting broadly cross reactive neutralizing antibodies (bcrnAbs) against HIV infections. In another aspect, the method of the invention can be utilized to produce vaccines against other human diseases, such as other viruses, cancer, and infectious micoorganisms, including bacteria, yeast, and protists, wherein the vaccines are capable of eliciting antibodies against the desired target.

The method is based on the observation that the amino acid sequences of known anti-HIV bcrnAbs bear a high extent of somatic mutational diversification (SMD) (about 20% difference) as compared to their germline immunoglobulin sequences, whereas antibodies against acute-infection viruses (e.g., SARS CoV and henipavirus) have a much lower SMD (about 1-6% difference) as compared to their germline counterpart sequences. Given the high degree of SMD in anti-HIV bcrnAbs and in view of the complexities of the mechanisms involved in B cell development, the elicitation of antibodies by HIV immunogens wherein the antibodies have sequences that are the same or similar to bcrnAbs could take years or longer, making it practically impossible to rely on such immunogens as vaccines, except perhaps in those individuals with already appropriately diversified B cells. Importantly, if the HIV or HIV-derived immunogens do not bind germline BCRs to begin with, such bcrnAbs might never even be elicited.

To overcome this hurdle, the method of the invention, in one aspect, provides a novel approach to bring about the eliciting of a desired antibody, e.g., a bcrnAb, against a target of interest, e.g., an HIV or cancer antigen target, by initially challenging the immune system with an immunogen (a “primary” immunogen) which elicits an “intermediate” antibody which bears only an intermediate degree of SMD and which only weakly binds the target of interest, e.g., HIV, and subsequently challenging the immune system with an immunogen of the target of interest, e.g, an HIV immunogen, e.g., gp160, gp140, gp120 or gp41 or fragments thereof. More in particular, the primary immunogen is introduced to elicit B cell receptors (BCRs) having an intermediate extent of SMD in the maturational pathway of an antibody of interest, e.g., a bcrnAb. The BCRs are then further diversified by the presence of a desired target immunogen, e.g., HIV immunogen, which elicits an immune response leading to the further mutation of the intermediate BCRs to generate BCRs having a specific or at least similar sequence of the antibody of interest, e.g., the bcrnAb of interest, e.g., an anti-HIV bcrnAb. Thus, the method of the invention advantageously provides a novel mechanism to elicit desirable antibodies (e.g., bcrnAbs) through the use a primary immunogen to elicit a population of BCRs along the maturational pathway of a desired antibody (e.g., a bcrnAb) which have an intermediate degree of somatic mutational diversification, which are then further mutated in the presence of a subsequent target immunogen, e.g., an HIV immunogen, to form BCRs having sequences that are the same or similar to a desired antibody (e.g., a bcrnAb, such as, an HIV-specific bcrnAb).

The present invention also provides methods for obtaining any of the vaccines, antigens, immunogens and/or antibodies required to make and use the present invention, as well as to the vaccines, antigens, immunogens and/or antibodies themselves. Moreover, the present invention provides methods for the treating of and/or the vaccinating against a human disease, e.g., a cancer, or infectious agent, e.g., HIV, by administering a therapeutically effective amount of a vaccine of the invention.

Thus, in one aspect, the present invention provides a vaccine effective to elicit a desired antibody, e.g., a broadly cross reactive neutralizing antibody (bcrnAb), against a target antigen comprising a primary immunogen and a secondary immunogen, wherein the primary immunogen is effective to elicit B cell receptors (BCRs) that are on the maturational pathway of the desired antibody and have an intermediate degree of somatic mutational diversity, and the secondary immunogen comprises an epitope of the desired antibody and is effective to further diversify the BCRs sufficient to form mature BCRs having the identical or substantially identical sequence as the desired antibody.

In another aspect, the present invention provides a method for vaccinating a subject against a disease comprising a target antigen, the method comprising co-administering a primary immunogen and a secondary immunogen, wherein the primary immunogen is effective to elicit B cell receptors (BCRs) that are on the maturational pathway of a desired antibody, e.g., a bcrnAb, specific for the target antigen and which have an intermediate degree of somatic mutational diversity, and the secondary immunogen contains an epitope of the desired antibody and is effective to further diversify the BCRs to form mature BCRs having the identical or substantially identical sequence as the desired antibody.

In still another aspect, the present invention provides a method for eliciting a desired antibody, e.g., a broadly cross reactive neutralizing antibody (bcrnAb), against a desired target antigen comprising co-administering a primary immunogen and a secondary immunogen, wherein the primary immunogen is effective to elicit B cell receptors (BCRs) that are on the maturational pathway of the desired antibody and have an intermediate degree of somatic mutational diversity, and the secondary immunogen contains an epitope of the desired antibody and is effective to further diversify the BCRs to form mature BCRs having the identical or substantially identical sequence as the desired antibody.

The desired target antigen of the invention can be an HIV antigen. The target antigen can also be an antigen of another disease, such as cancer.

The desired bcrnAb can be a known HIV-specific bcrnAb, or a bcrnAb specific to another disease agent such as a cancer cell or antigen.

The known HIV-specific bcrnAb can be b12, 2F5, 4E10, 2G12, m14, m18, m43, m44, m46, m47 or m48.

The BCRs with an intermediate degree of somatic mutational diversification can have between 1 and 5 mutations relative to the corresponding germline immunoglobulin amino acid sequence.

The BCRs with an intermediate degree of somatic mutational diversification can also have between 5 and 10 mutations relative to the corresponding germline immunoglobulin sequence.

The BCRs with an intermediate degree of somatic mutational diversification can have between 10 and 20 mutations relative to the corresponding germline immunoglobulin sequence.

The amino acid sequences of the mature BCRs can be at least 90% identical to the amino acid sequence of the desired bcrnAb.

The secondary immunogen can be an HIV-specific immunogen, such as Env, gp160, gp140, gp120, gp41 or fragments thereof.

The secondary immunogen can be an cancer-specific immunogen.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 provides a schematic representation of the maturational pathway of an antibody starting from the binding of an immunogen to the germline B cell receptor (BCR) to matured antibody through an intermediate antibody. The figure illustrates the activation of B cells containing germline BCR by the binding of a primary immunogen that leads to somatic hypermutation or somatic mutational diversification, which forms BCRs having several or an intermediate number of mutations and which are on the maturational pathway of the desired antibodies. The intermediate BCRs cross-react with a secondary immunogen (e.g., HIV gp160, gp140, gp120 or gp41) leading to further diversification of the B cells and BCRs until the final desired matured antibody is elicited.

FIG. 2 is a graphical depiction demonstrating that germline 2G12 antibody does not bind Env gp140.

FIG. 3 is a graphical depiction demonstrating that germline 2F5 antibody does not bind to HIV-1 89.6 gp41.

FIG. 4 is a graphical depiction demonstrating that germline b 12 (in both (A) scFv and (B) IgG formats) does not bind Env antigen from different HIV strains. Mature b12 in scFv format, germline b12 in both scFv and IgG formats, and an irrelevant IgG were tested for their bindings to four different HIV Envs as indicated on X axis. BSA was included as a negative control. The single concentration used for each antibody was 2.5 μM.

FIG. 5 provides a chart comparing the binding of relative binding strength of cross-reactive antibodies (Abs) and their corresponding germline antibodies to their cognate antigens.

FIG. 6 provides a chart showing the number of mutations in known HIV-1 broadly cross-reactive neutralizing antibodies and the estimated number of years it is predicted to take to elicit the antibodies.

FIG. 7 shows various features of corresponding germ-like antibodies, including germ line-like V(D)J gene usage, CDR3 sequence, and variable gene mutation.

FIG. 8 shows detectable bindings of germline-like X5, m44, and m46 antibodies in scFv format to Env. Bal gp120-CD4 fusion protein was coated on a 96 well ELISA plate for detection of scFv X5 binding, whereas 89.6 gp140 was coated for detection of scFv m44 and m46 bindings at indicated concentrations (“concentration (nM)”). Mature (diamond symbols) and germline-like (square symbols) antibodies were compared. See Example 9 for details.

FIG. 9 depicts lack of binding of germline-like b12, 2G12, and 2F5 antibodies in scFv format. Bal gp120 was coated for detection of b12 binding and 89.6 gp140 was coated for detection of binding by both scFv 2G12 and 2F5. Mature (diamond symbols) and germline-like (square symbols) formats were compared. See Example 9 for details.

FIG. 10 demonstrates a lack of binding of germline-like b12, 2G12, and 2F5 antibodies in Fc fusion protein format to Env. Bal gp120 was coated for detection of mature and germline-like scFv-Fc b12 binding and 89.6 gp140 was coated for detection of binding by mature scFv and germline-like scFv-Fc 2G12 and 2F5. See Example 9 for details.

FIG. 11 shows detectable bindings of germline-like m44 and m46 antibodies in Fc fusion protein format to Env. Env 89.6 gp140 was coated for detection of binding by scFv-Fc m44 and m46 fusion proteins. See Example 9 for details.

FIG. 12 depicts binding characteristics of the scFv X5 in various forms including mature, germline, and hybrids between various heavy and light chains. (a) and (c) depict gel analysis of purified mature, germline and hybrid scFv X5. In (a), M, molecular weight marker, 1 and 2 are mature and germline scFv X5 respectively. In (c), M, molecular weight marker, 1 is the hybrid scFv between mature X5 heavy chain and germline X5 light chain, and 2 is the hybrid between matured X5 heavy chain and matured b12 heavy chain. (b) and (d) show bindings of purified proteins shown in (a) and (c), respectively, to bal gp120-CD4. Abbreviations in this and subsequent figures are as following: “math,” matured heavy chain; “germl,” germline light chain; “matl,” matured light chain; and “germh,” germline heavy chain.

FIG. 13 shows inhibition of pseudovirus infection by mature and germline scFv X5. Nine HIV Env-pseudotyped viruses were tested with a single concentration of both X5 original and germline as described in Materials and Methods of Example 10. The concentration of scFv used is 600 nM. The names of the Envs used are shown on the X-axis and the numbers on the Y-axis represent the percentages of the pseudovirus activities. For each isolate, the bars represent the percentage of activities of the viruses treated with PBS only (left columns), scFv X5 original (middle columns), and germline (right columns).

FIG. 14 depicts the determination of the IC50s of the mature and germline scFv X5 against representative HIV isolates (IIIB (A.), GXC-44 (B.), and Bal (C.)). Viruses pseudotyped with Envs from M and T tropic viruses from B Glade as well as one from A Glade were used in neutralization assay.

FIG. 15 shows the identification of b12 intermediate binders. Point mutations were introduced back to the H2 and adjacent frame work of germline b12. The resultant mutants were expressed and purified both as scFv and scFv-Fc. (a) provides the gel analysis of the purified scFv and scFv-Fc. The gel is defined as: M, molecular weight marker. Samples 1-9 are (1) original, (2) germline, (3) A52P/G53Y, (4) G53Y, (5) math/germl, (6) germh/matl, (7) G53D, (8) A52P/T57K, and (9) A52P. Samples 10-15 are (10) mature b12-Fc, (11) germline b12-Fc, (12) A52P/G53Y-Fc, (13) G53Y-Fc, (14) math/germl-Fc, and (15) germh/matl-Fc. (b) shows the bindings by the selected scFv analyzed against bal gp120 in an ELISA. Two concentrations, including 8 (bar closer to Y-axis) and 2.7 μM of each scFv, were used. (c) shows the control, BSA, as an antigen which was included as a specificity control. The maximum value of Y-axis was set at 0.5 to reflect the weak bindings. Bindings of both mature b12 and A52P/G53Y b12 reached saturation at both concentrations and were indicated.

FIG. 16 depicts a determination of the strength of binding by various formats of b12. ELISAs were performed using bal gp120 as the antigen. Various scFv (a) and scFv-Fc (b) b12 as indicated were analyzed for their bindings. (c) shows the competition ELISA between various scFv b12 and the original scFv-Fc b12. Fixed amount of various scFv b12 at 20 μg was pre-mixed with increasing amount of original scFv-Fc b12 in 100 μl of blocking buffer and applied to ELISA plate coated with bal gp120. The amount of bound scFv was measured using anti-his-HRP. (d) shows the specific competition between sCD4 and various forms of b12 in binding to bal gp120. Fixed amount of sCD4 at 2 μg was mixed with increasing amount of various b12-Fc fusion protein in 100 μl of blocking buffer and added to ELISA plate coated with bal gp120. The bound sCD4 was detected with anti-his-HRP.

FIG. 17 shows the inhibition of pseudovirus infection by various scFv-Fc b12. Nine HIV Env-pseudotyped viruses were tested with a panel of scFv-Fc b12 variants. The original-Fc b12 was used at a concentration of 0.3 μM, while all the other b12-Fc fusion variants including the germline-Fc b12 were used at a concentration of 2 μM. For each isolate, the bars represent the percentage of activities of the viruses treated with PBS only, b12-Fc, math/germl-Fc, A52P/G53Y-Fc, germh/matl-Fc, G53Y-Fc, and germline-Fc sequentially, with the PBS treated sample closet to the Y-axis.

FIG. 18 depicts the binding of various b12-Fc proteins to the surface antigens of three human cell lines. (a) Germline-Fc, G53Y-Fc, A52P/G53Y-Fc and mature-Fc b12 proteins were used at a concentration of 1 μM in the flow cytometry assay as described in cell lines (1) 293T, (2) SK-N-AS and (3) HOS. (b) Germline-Fc, math/germl-Fc, germh/matl-Fc and original-Fc were further compared in the flow cytometry assay in cell line SK-N-AS. The concentration used remained at 1 μM. The numbers on the X-axis represent the binding intensity and the numbers on the Y-axis represent the number of cells.

### DETAILED DESCRIPTION OF THE INVENTION

It will be appreciated that the quest for an AIDS vaccine has been a major challenge and of high priority for governmental and private institutions and industry alike. A major initiative has been the search for vaccine immunogens that could elicit broadly cross-reactive HIV neutralizing antibodies (bcrnAbs). Such antibodies are rarely elicited in HIV-infected humans, and only several such monoclonal bcrnAbs are known including b12, 2F5, 4E10, 2G12, m14, m18, m43, m44, m46, m47 and m48. The current paradigm is that an appropriate vaccine immunogen containing the epitopes of these bcrnAbs could lead to their elicitation in vivo. However, in spite of the tremendous amount of work and billions of dollars spent to date, this approach has failed.

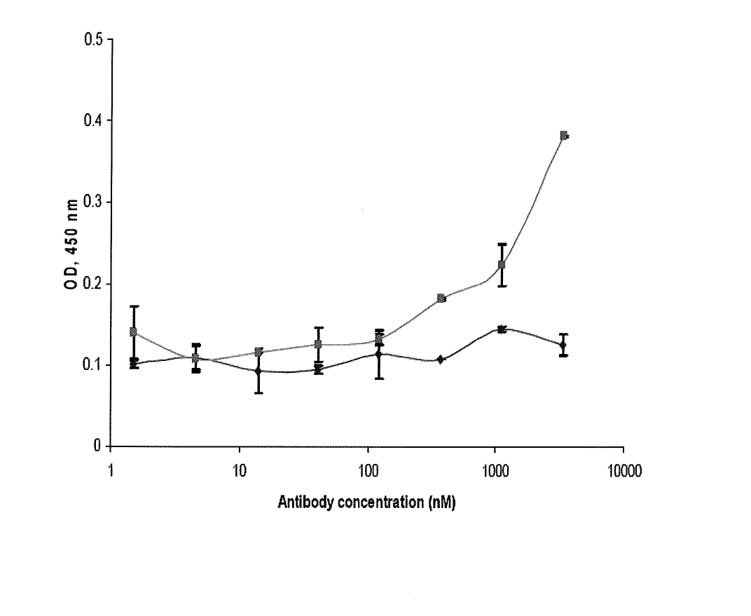

The present inventor has observed by sequence analysis of HIV-specific bcrnAbs and antibodies against other viruses (e.g., viruses causing acute infection, such as henipaviruses and the SARS CoV) against their corresponding germline immunoglobulin sequences, that the HIV-specific bcrnAbs contained a substantially higher degree of somatic mutational diversification (SMD) (i.e., the number of mutations as compared to the corresponding closest germline immunoglobulin sequence) than the counterpart antibodies elicited against the other viruses. The SMD of the HIV bcrnAbs was up to about 20%, whereas the SMD of antibodies elicited against the other viruses was only between 1-6%. It was also observed that the corresponding germline antibodies of known bcrnAbs, e.g., 2G12, 2F5 and b12, did not bind to the HIV Env (see e.g., FIGS. 2-4).

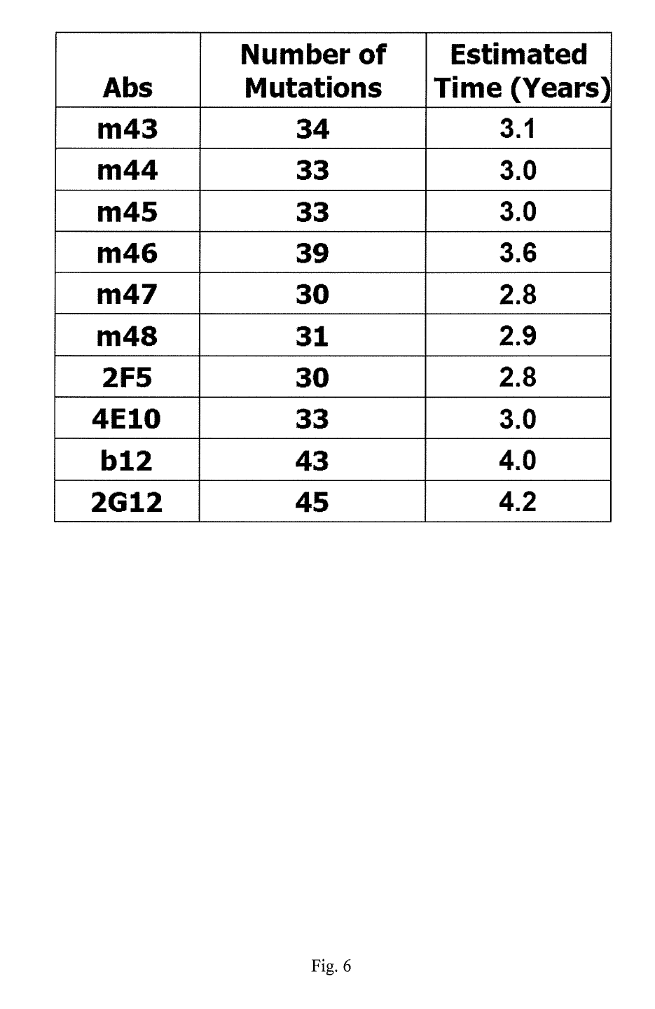

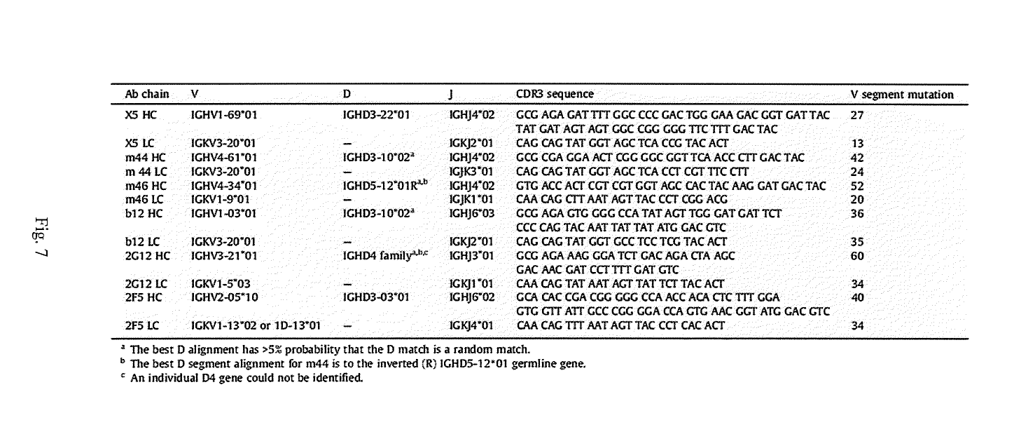

Given the high degree of SMD in anti-HIV bcrnAbs (see e.g., FIG. 6) and in view of the complexities of the mechanisms involved in B cell development, the elicitation of antibodies by HIV immunogens wherein the antibodies have sequences that are the same or similar to bcrnAbs could take years or longer, making it practically impossible to rely on such immunogens as vaccines, except perhaps in those individuals with already appropriately diversified B cells. Such complexities involved in B cell development can include the B cell activation and maturation processes (e.g., development of mature naïve B cells, B cell activation, clonal proliferation, differentiation and affinity maturation), the limiting number of B cells in humans (about 10¹⁰ total and about 10³ per germinal center where the hypermutation/affinity maturation occurs) and the enormous space of possible antibody sequences (>10¹⁰⁰ for 20% mutated amino acid residues) which may increase exponentially with the number of mutations.

Importantly, the corresponding germline (or close to germline) antibodies of some of the known bcrnAbs may not bind vaccine immunogens based on the HIV Env, or B cells expressing such germline antibodies could be deleted or anergized during the B cell development; thus, precluding the initiation of an immune response leading to their elicitation. This lack of germline antibodies capable of binding those epitopes (i.e., the source of the “holes” in our germline repertoire) is likely to be used by HIV to evade immune responses against functionally important epitopes.

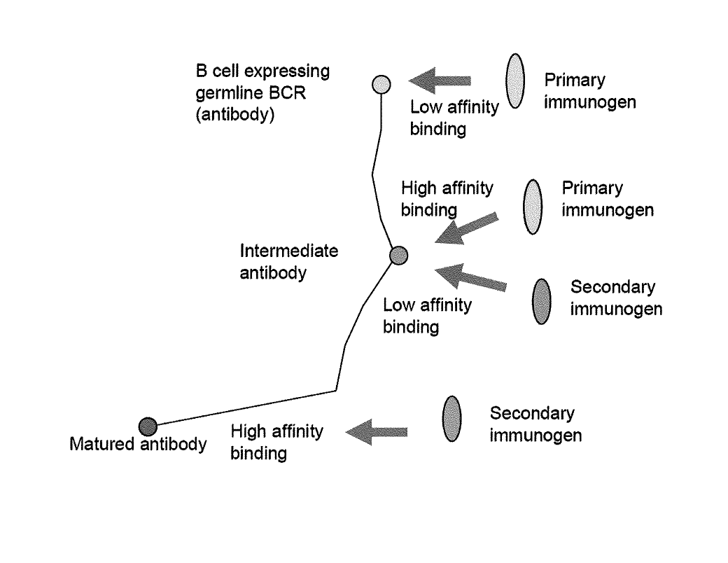

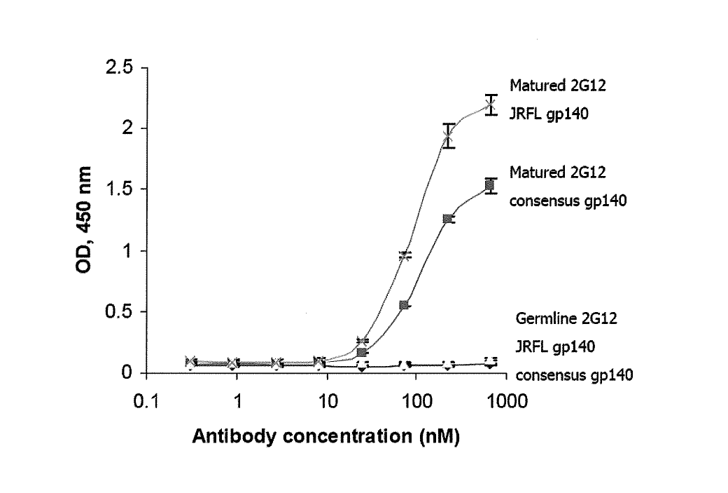

The method of the present invention provides, in one aspect, it is believed for the first time, a novel method to achieve the generation of highly diversified antibodies with sequences identical or close to those of known bcrnAbs, e.g., HIV-specific bcrnAbs, that cannot be elicited by currently used methods and which are rarely elicited in some individuals even after prolonged periods of time. The method generally involves eliciting one or more B cell associated antibodies (receptors) (BCRs) by challenging with a first immunogen (termed here primary immunogen, FIG. 1), wherein the BCRs are characterized as having an intermediate degree of SMD and which are on a maturational pathway of a desired bcrnAb. The intermediate BCRs are further mutated by challenging with a second (termed here secondary) immunogen (see FIG. 1), preferably an immunogen, e.g., an HIV-specific immunogen, that contains epitopes of a desired bcrnAb, e.g., an HIV-specific bcrnAb. Thus the intermediate BCRs are cross-reactive to both the primary and the secondary antigens. The affinity of the intermediate BCRs to the primary antigens is generally high, whereas the affinity of the intermediate BCRs to the secondary antigen is low. It will be appreciated that the BCRs are membrane-associated and therefore polyvalent, and an effective affinity (avidity) is measured by characterizing the strength of their binding to oligomeric antigens.

Thus, in one aspect, the invention provides a new vaccine (e.g., an HIV-specific or cancer-specific vaccine) that comprises two or more vaccine immunogens that are used simultaneously or sequentially, wherein a first immunogen (primary immunogen) is administered to elicit BCRs with an intermediate extent of SMD on the maturational pathway of a bcrnAb. A second immunogen (secondary immunogen), which contains epitopes of a desired bcrnAb (e.g., for HIV, an immunogen based on Env or a fragment thereof), is administered simultaneously or sequentially (shortly) after the first immunogen, which acts to further diversify the intermediate BCRs until the sequence of the BCR and its secreted antibodies is the same as or is closely similar to the desired HIV-specific bcrnAb. Because the second immunogen acts at a point at which an intermediate diversification of the BCRs has already been attained, less time is required to obtain antibodies with sequences identical or close to those of the desired bcrnAbs. Importantly, in the case of HIV, if the Env-based immunogens do not bind germline BCRs corresponding to those of HIV-specific bcrnAbs, the use of a primary immunogen makes possible elicitation of such antibodies which otherwise would be impossible to elicit as numerous experiments have failed to demonstrate such elicitation for the past decades.

Without wishing to be bound by theory, an important concept underlying the invention is that by providing additional information to the immune system (i.e., by virtue of the first immunogen), the generation of the immune response is funneled through a relatively restricted number of BCR mutational pathways, thereby significantly reducing the combinatorial explosion which the immune system faces attempting to stochastically identify possible pathways leading to the desired bcrnAbs in a huge space of possible combinations (e.g., estimated at more than 10¹⁰⁰ possible sequences for 20% mutations of 200 positions of 20 different amino acid residues although not all those sequences are productive; however if only 20-30 positions corresponding to the CDRs are mutated still the space of possible antibodies is enormous).

In general, the first immunogen can be designed based on a knowledge of the pathways of SMD. By identifying one or several antibodies with intermediate SMD (e.g., which cross-react with the Env containing the epitope of the desired bcrnAb being on the maturational pathway of that antibody), primary immunogens (i.e., the first, primary, immunogens) can be obtained based on structures that contain the epitopes recognized by the intermediate Abs. The second (secondary) immunogen can be based on an a target immunogen, e.g., an HIV-specific immunogen, which contains epitopes recognized by a known bcrnAb, such as an immunogen based on the HIV-1 envelope glycoprotein or fragment thereof, e.g., gp120 or gp41 and fragments or derivatives thereof.

The invention further provides methods and compositions for vaccinating against and/or treating a subject with an HIV infection or other disease, e.g., cancer, by administering a therapeutically effective amount of the vaccine (e.g., primary and secondary immunogens) of the invention or a composition thereof. Methods and guidance are provided herein to obtain each of the components required to carry out the vaccination/treatment methods of the invention, including, obtaining the first and second immunogens and antibodies of the invention.

It is to be understood that present invention as described herein is not to be limited to the particular details set forth herein regarding any aspect of the present invention. It is also to be understood that the terminology used herein is for the purpose of describing particular embodiments only, and is not intended to limit the scope of the present invention.

### DEFINITIONS

Unless defined otherwise, all technical and scientific terms used herein have the meaning commonly understood by one of ordinary skill in the art to which this invention belongs.

As used herein, the term “antibody” is meant to refer to immunoglobulin molecules (e.g., any type, including IgG, IgE, IgM, IgD, IgA and IgY, and/or any class, including, IgG1, IgG2, IgG3, IgG4, IgA1 and IgA2) isolated from nature or prepared by recombinant means or chemically synthesized. The terms “antibody” and “immunoglobubin” can be used interchangeably throughout the specification, unless indicated otherwise.

As used herein, the term “antibody fragment” is meant to refer to a portion of a whole antibody which retains the ability to exhibit antigen binding activity or immunogenicity. Examples include, but are not limited to, Fv, disulphide-linked Fv, single-chain Fv, Fab, variable heavy region (V_{H}), variable light region (V_{L}), and fragments of any of the above antibody fragments which retain the ability to exhibit antigen binding activity, e.g., a fragment of the variable heavy region V_{H} retains its ability to bind its antigen.

For preparation of monoclonal or polyclonal antibodies, any technique known in the art can be used (see, e.g., Kohler and Milstein, Nature 256:495497 (1975); Kozbor et al., Immunology Today 4:72 (1983); Cole et al., pp. 77-96 in Monoclonal Antibodies and Cancer Therapy, Alan R. Liss, Inc. (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

The term “immunoassay” is meant to refer to an assay that uses an antibody to specifically bind an antigen. The immunoassay is characterized by the use of specific binding properties of a particular antibody to isolate, target, and/or quantify the antigen.

As used herein, the terms “biological sample” or “patient sample” as used herein, is meant to refer to a sample obtained from an organism or from components (e.g., cells) of an organism. The sample can be of any biological tissue or fluid. The sample may be a clinical sample which is a sample derived from a patient. Such samples include, but are not limited to, sputum, blood, serum, plasma, blood cells (e.g., white cells), tissue samples, biopsy samples, urine, peritoneal fluid, and pleural fluid, saliva, semen, breast exudate, cerebrospinal fluid, tears, mucous, lymph, cytosols, ascites, amniotic fluid, bladder washes, and bronchioalveolar lavages or cells therefrom, among other body fluid samples. The patient samples may be fresh or frozen, and may be treated, e.g. with heparin, citrate, or EDTA, or other suitable treatment known in the art. Biological samples may also include sections of tissues such as frozen sections taken for histological purposes. Samples can be infected with HIV.

As used in this invention, the term “epitope” is meant to refer to any antigenic determinant on an immunogen, e.g., an primary immunogen, or a gp120 or gp41 protein, to which an antibody binds through an antigenic binding site. Determinants or antigenic determinants on an antigen usually consist of chemically active surface groupings of molecules such as amino acids or sugar side chains and usually have specific three dimensional structural characteristics, as well as specific charge characteristics. The epitope can be a CD4-inducible epitope, i.e., an epitope that becomes available or accessible only upon CD4 binding or during fusion.

As used herein, the term antibody that “specifically (or selectively) binds to” or is “specific for” or is “specifically (or selectively) immunoreactive with” a particular polypeptide or an epitope on a particular polypeptide is one that binds to that particular polypeptide or epitope on a particular polypeptide without substantially binding to any other polypeptide or polypeptide epitope. Antibody affinity for antigens can be measured by enzyme linked immunosorbent assay (ELISA). Alternatively, an antibody that specifically binds to an oligomeric antigen, in accordance with this invention, refers to the binding of an antigen by an antibody or fragment thereof with a dissociation constant (K_{d}) of 1 μM or lower, as measured by surface plasmon resonance analysis using, for example, a BIACORE surface plasmon resonance system and BIACORE kinetic evaluation software (e.g., version 2.1). The affinity or dissociation constant (K_{d}) for a specific binding interaction is preferably about 500 nM or lower, more preferably about 300 nM or lower and preferably at least 300 nM to 50 pM, 200 nM to 50 pM, and more preferably at least 100 nM to 50 pM, 75 nM to 50 pM, 10 nM to 50 pM.

The term “gp160” refers to the human immunodeficiency virus-1 envelope glycoprotein gp160 kDa (or its corresponding gene), which is processed to form the 120 kDa (gp120) subunit and the 41 kDa (gp41) subunit.

The terms “gp120” or “gp120 subunit”, as used herein, is meant to refer to the human immunodeficiency virus-1 envelope glycoprotein gp120. The terms “gp120 variant”, “gp120 mutant”, or “gp120 derivative” refers to a protein which is characterized by: (1) having an amino acid subsequence that has greater than about 60% amino acid sequence identity, 65%, 70%, 75%, 80%, 85%, 90%, preferably 95%, 96%, 97%, 98%, 99% or greater amino acid sequence identity, to the sequence of HIV-1 gp120. The nucleic acid and amino acid sequences of HIV gp-120 are readily available to the public through the HIV sequence database on the world wide web at hiv.lanl.gov/content/sequence/HIV/mainpage.html; (2) binding to antibodies, e.g., polyclonal antibodies, raised against an immunogen comprising an amino acid sequence of HIV-1 gp120; (3) specifically hybridizing under stringent hybridization conditions to a nucleic acid sequence encoding HIV-1 gp120 and (4) having a nucleic acid sequence that has greater than about 85%, preferably greater than about 90%, 95%, 98%, 99%, or higher nucleotide sequence identity to the nucleic acid sequence encoding HIV-1 gp120.

The terms “gp41” or “gp41 subunit”, as used herein is meant to refer to the human immunodeficiency virus-1 envelope glycoprotein gp41. The terms “gp41 variant”, “gp41 mutant”, or “gp41 derivative” refers to a protein which is characterized by: (1) having an amino acid subsequence that has greater than about 60% amino acid sequence identity, 65%, 70%, 75%, 80%, 85%, 90%, preferably 95%, 96%, 97%, 98%, 99% or greater amino acid sequence identity, to the sequence of HIV-1 gp41. The nucleic acid and amino acid sequences of HIV gp-41 are readily available to the public through the HIV sequence database on the world wide web at hiv.lanl.gov/content/sequence/HIV/mainpage.html; (2) binding to antibodies, e.g., polyclonal antibodies, raised against an immunogen comprising an amino acid sequence of HIV-1 gp41; (3) specifically hybridizing under stringent hybridization conditions to a nucleic acid sequence encoding HIV-1 gp41 and (4) having a nucleic acid sequence that has greater than about 85%, preferably greater than about 90%, 95%, 98%, 99%, or higher nucleotide sequence identity to the nucleic acid sequence encoding HIV-1 gp41.

As used herein, the term “regulatory sequences” refers to those sequences, both 5′ and 3′ to a structural gene, that are required for the transcription and translation of the structural gene in the target host organism. Regulatory sequences include a promoter, ribosome binding site, optional inducible elements and sequence elements required for efficient 3′ processing, including polyadenylation. When the structural gene has been isolated from genomic DNA, the regulatory sequences also include those intronic sequences required for splicing of the introns as part of mRNA formation in the target host.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2′-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. The probes can be directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

By “host cell” is meant a cell that contains an expression vector and supports the replication or expression of the expression vector. Host cells can be mammalian cells such as CHO, HeLa and the like, e.g., cultured cells, explants, and cells in vivo, or bacterial host cells.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, 65%, 70%, 75%, 80%, preferably 85%, 90%, 91%, 92%, 93%, 94%, 95%, 96%, 97%, 98%, 99% or higher identity to a reference sequence when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of the antibodies and antigens of the invention, BLAST and BLAST 2.0 algorithms and the default parameters discussed below can be used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

BLAST and BLAST 2.0 algorithms are described in Altschul et al., Nuc. Acids Res. 25:3389-3402 (1977) and Altschul et al., J. Mol. Biol. 215:403410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nln.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915 (1989)) alignments (B) of 50, expectation (E) of 10, M=5, N4, and a comparison of both strands.

The BLAST algorithm also performs a statistical analysis of the similarity between two sequences (see, e.g., Karlin and Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tm for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tₘ 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

As used herein, the term “vaccine” is meant to encompass any immunogenic composition that is capable of inducing an immune response in a subject. The vaccine can include one or more immunogens, e.g., an primary immunogen together with a secondary immunogen, e.g., HIV antigen immunogen. By immune response is meant to include responses that result in at least some level of immunity in the treated subject, where the subject was treated with a composition of the present invention.

As used herein, the term “immunogen” is any substance or organism that provokes an immune response (produces immunity) when introduced into the body. The modern definition encompasses all substances that can be recognized by the adaptive immune system. Immunogens are those substances that elicit a response from the immune system. By contrast, “antigens” are defined as substances that bind to specific antibodies and can cause an immunogenic response. Not all antigens produce an immunogenic response, but all immunogens are antigens (Immunobiology, Janeway and Travers, 1994).

As used herein, the term “intermediate antibodies” define antibodies (including B cell associated antibodies, i.e., BCRs) with intermediate somatic mutational diversification on the maturational pathway of an antibody from a germline antibody to a maturated antibody (see FIG. 1). An intermediate antibody can have one or more mutated amino acid residues compared to the germline antibody but has fewer mutated residues compared to the mature antibody. Preferably, the intermediate antibody has between 1% to 90%, or between 10% to 80%, or between 20% to 70% or about 40% to 60% or even about 50% of the mutations of the corresponding mature antibody.

The term “somatic mutational diversification (SMD)” is a measure of the number of mutated amino acid residues compared to the germline and is a consequence of the natural B cell diversification processes, including affinity maturational processes in which the B cell undergoes hypermutation in a germinal center in the presence of an antigen. It is expressed as the percentage of that number compared to the total number of amino acid residues in the sequences. Typically the number of amino acids encoded by the VH gene is used to measure the SMD because usually the heavy chain variable region is a major determinant of the antibody specificity and the VH gene encodes most of the amino acid residues of the heavy chain variable region.

A primary (intermediate) immunogen is an antigen that binds to an intermediate antibody (or BCR) and can elicit that antibody in vivo.

A secondary immunogen is an antigen that comprises an epitope of a desired bcrnAb or other desired antibody that is sought to be elicited by the vaccine of the invention.

As used herein and in the appended claims, the singular forms “a,” “and,” and “the” include plural reference unless the context clearly dictates otherwise. Thus, for example, reference to “a gene” is a reference to one or more genes and includes equivalents thereof known to those skilled in the art, and so forth.

### B Cell Development

Without wishing to be bound by theory, the basic principles of B cell development are useful to understand in the context of the present invention. B cells are lymphocytes that play a large role in the humoral immune response. The principal functions of B cells are to make antibodies against antigens, perform the role of Antigen Presenting Cells (APCs) and eventually develop into memory B cells after activation by antigen interaction. B cells are an essential component of the adaptive immune system.

B cell development begins with the formation of immature B cells in the bone marrow. Immature B cells are produced in the bone marrow of most mammals. After reaching the IgM+immature stage in the bone marrow, these immature B cells migrate to the spleen, where they are called transitional B cells, and some of these cells differentiate into mature B lymphocytes. B cell development occurs through several stages, each stage representing a change in the genome content at the antibody loci. An antibody is composed of two identical light (L) and two identical heavy (H) chains, and the genes specifying them are found in the ‘V’ (Variable) region and the ‘C’ (Constant) region. In the heavy-chain ‘V’ region there are three segments; V, D and J, which recombine randomly, in a process called VDJ recombination, to produce a unique variable domain in the immunoglobulin of each individual B cell. Similar rearrangements occur for light-chain ‘V’ region, which include only V and J sections.

Various types of B cells are recognized during the developmental pathway. The progenitor B cells contain germline H (heavy chains) genes and germline L (light chains) genes. The early pro-B cells undergo D-J rearrangement on the H chain genes. The late pro-B cells undergo V-DJ rearrangement on the H chain genes. The large pre-B cells contain a VDJ rearranged H chain genes and germline L genes. The small pre-B cells undergo V-J rearrangement on the genes encoding the L chains. The immature B cells have VJ rearranged L chain genes and VDJ rearranged H chain genes. In addition, IgM receptors begin to be expressed in the immature B cells. The mature B cells express both IgM and IgD.

When the B cell fails in any step of the maturation process, the cell undergoes apoptosis, i.e., clonal deletion. If the B cell recognizes self-antigen during the maturation process, the B cell will become suppressed (known as anergy) or will undergo apoptosis (also termed negative selection). B cells are continuously produced in the bone marrow. When a B cell receptor (BCR) (i.e., IgM) on the surface of the cell matches the detected antigens present in the body, the B cell proliferates and secretes a free form of those receptors (antibodies) with identical binding sites as the ones on the original cell surface. After activation by antigen or by T-cell activation, the B cell proliferates and forms B memory cells.

It will be appreciated that B cells exist as clones. Thus, the antibodies produced by their differentiated progenies can recognize and/or bind the same components (epitope) of a given antigen. This has important consequences, most notably, the phenomenon of immunogenic memory relies on this clonality of B cells. The great diversity in immune response comes about because there are up to 10⁹-10¹⁹ clones with that many specificities for recognizing antigens. A single B cell or a clone of cells with shared specificity upon encountering its specific antigen (rather, the epitope), divides many times to produce many B cells, most of which differentiate into plasma cells that can secrete antibodies into blood that bind the same epitope that initialized the proliferation, while a very small minority survive as memory cells that can again recognize only the same epitope, and when that happens would divide further to produce more plasma and memory cells. However, with each such cycle, the number of surviving memory cells increases. This also is accompanied by the process of affinity maturation, which increases the sum total of epitopes that can be recognized by the related clones through random mutations in the epitope binding (and recognizing) portions of these cell-associated antibodies. With repeated exposures to the same antigen, a host will produce antibodies of successively greater affinities. Such a secondary response can elicit antibodies with several log-fold greater affinity than in a primary response.

Affinity maturation is thought to involve two interrelated processes, occurring in the germinal centers of the secondary lymphoid organs. In the first process, somatic hypermutation (SHM), polymorphisms in the variable, antigen-binding coding sequences (known as complementarity-determining regions) of the immunoglobulin genes clonally accumulate with repeated stimuli. These polymorphisms stochastically alter the binding specificity and binding affinities of the resultant antibodies produced by progeny. In the second process, clonal selection, B cells that have undergone SHM must compete for limiting growth resources, including the availability of antigen. The follicular dendritic cells (FCDs) of the germinal centers present antigen to the B cells, and only the B cell progeny with the highest affinities for antigen will be selected to survive. B cell progeny that have undergone SHM, but bind antigen with lower affinity will be outcompeted and deleted.

In some cases when the germline BCR can bind the immunogen with relatively high affinity only few mutations and rounds of selections are required to reach a high-affinity matured antibodies, e.g., immunogens (typically envelope glycoproteins) of some viruses which cause acute infections. However, for other immunogens e.g. structures containing the epitopes of known HIV-1-specific bcrnAbs, many more mutations (high degree of somatic mutational diversification (SMD)) and rounds of selection are required to elicit high-affinity matured antibodies. If the immunogen (e.g. HIV Env) does not bind to the germline BCR corresponding to the matured antibody then stochastically irrelevant immunogens that do bind that germline could cause partial SMD to an intermediate BCR that could bind that immunogen (the HIV Env) cross-reactively (and typically with low affinity) and be further mutated (diversified) to a high-affinity matured antibody.

### Discovery

Not wishing to be bound by theory, the present invention is based on the discovery that bcrnAbs against HIV contain a significantly higher number of mutations over the germline immunoglobulin sequences as compared to bcrnAbs against various other viruses. In more detail, during the last several years, the present inventor and his associates had identified and characterized many hmAbs (human monoclonal antibodies) against HIV-1, some of which exhibit potent cross-reactive neutralizing activity against primary HIV-1 isolates from different clades (Zhang et al., 2003; Moulard et al., 2003; Zhang et al., 2004a; Zhang et al., 2004b; Zhang et al., 2004c; Choudhry et al., 2006; Zhang et al., 2006; Choudhry et al., 2007; Zhang & Dimitrov, 2007) (Zhang et al., AIDS Vaccine 2007, Late Breaker presentation). Recently, the inventor and his associates have also identified and characterized a number of hmAbs against the SARS CoV (Prabakaran et al., 2006a; Zhu et al., 2007), Hendra and Nipah viruses (Zhu et al., 2006a), and several other microbes causing acute infections. Some of these antibodies exhibit potent cross-reactive neutralization of SARS CoV isolates from humans and animals (Zhu et al., 2007) and to both henipaviruses, Nipah and Hendra (Zhu et al., 2006a).

The identification of many hmAbs against various infectious agents has provided a unique opportunity to analyze and compare their antibody sequences.

It was discovered that there existed a significant difference in the extent of immunoglobulin gene maturation between bcrnAbs against HIV and bcrnAbs against viruses causing acute viral infections, including SARS CoV, Hendra and Nipah viruses. The large extent of maturation of known HIV-specific bcrnAbs contrasted to the only few amino acid residue changes from the germline of bcrnAbs against viruses causing acute infections. The potent bcrnAbs against SARS CoV and henipaviruses were selected by screening of a large non-immune IgM antibody library (derived from ten healthy volunteers) against the respective Envs, thus mimicking to a certain extent in vivo immunization (screening of phage display libraries has been previously proposed as an in vitro method mimicking in vivo immunization (Parren et al., 1996)). Using the same library and screening methodology against the HIV Env resulted in weakly neutralizing non-cross reactive antibodies. Previous attempts to select HIV-specific antibodies by use of non-immune libraries have also resulted in antibodies with modest neutralizing activity and limited breadth of neutralization (Louis et al., 2005; Miller et al., 2005).

These findings indicate that during lengthy chronic infections, HIV has evolved to protect its most vulnerable but functionally important conserved structure, including the CD4 binding site and gp41 membrane proximal external region (MPER) by decreasing the probability to encounter B cell receptors (BCRs) that can bind to those structures. One possibility is that HIV has evolved strategies to use epitopes important for its function e.g. for its entry into cells, that do not bind to germline antibodies i.e. the virus utilizes existing “holes” in the human germline BCR repertoire (although the human germline BCR repertoire is large it is still much smaller than all antibodies that could bind existing epitopes).

Another possible scenario is that the HIV conserved epitopes mimic self proteins, and during the B cell development deletion or suppression of such B cells or BCR editing occurs in most subjects leads to lack or limited availability of B cells expressing BCR that can bind to some or all conserved HIV epitopes. In this scenario, one can hypothesize that such mature B cells expressing Ig antigen receptors capable of binding conserved epitopes are deleted or suppressed or their BCR edited. Yet another possible scenario is that HIV has evolved mechanisms to suppress the function of such B cells without mimicking self proteins, but by rather directly inducing mechanisms of tolerance control of these antibodies. In any case such B cells would be lacking or functionally inactive. In some rare cases of individuals with high titers of bnAbs the long period of infection likely has allowed extensive somatic hypermutation of some of the B cell clones to mature to cells producing bnAbs and/or has allowed such clones to escape tolerance because of chronic HIV infection inducing dysregulated immune function or already having autoimmune disease.

According to the new concept it is also possible that such individuals could have already had immunogens corresponding to the primary immunogen in this invention which would elicit intermediate BCR which could be further mutated to the bcrnAbs. Such individuals with high levels of bnAbs, though not having clinical autoimmune disease, could be predisposed by genetic or epigenetic mechanisms to having the ability to make polyspecific antibodies that others cannot make. It is also possible that both scenarios are operating and HIV uses both fundamentally different ways for protection of its precious conserved vitally important structures: some—by hiding them and others—by mimicking self proteins; some epitopes could be protected by both mechanisms. An example, based on preliminary studies described below, for hiding epitopes but not mimicking self proteins is likely to be the case of so-called CD4-induced epitopes. An example for not hiding but mimicking self proteins could be the gp41MPER. Thus in either case the probability of making bnAbs against HIV would be low. According to this invention another strategy that HIV may use is to find “holes” in the human BCR germline repertoire, i.e., lack of germline BCR that could bind to functionally important epitopes on the Env and initiate immune response leading to elicitation of bcrnAbs.

In contrast, viruses causing acute infection, such as SARS CoV, have not evolved such protective mechanisms, the antibodies that can potently neutralize them are similar to those with germline sequences and they can be quickly and easily elicited without the need for extensive somatic mutations which could contribute to the quick and efficient elicitation of neutralizing antibodies in humans and animals, and the success of candidate vaccines against SARS in various animal models (Zhu et al., 2006b). Note that similar to HIV, the SARS CoV is an RNA virus, that can easily mutate and could escape neutralization if provided with sufficient time for evolution of appropriate escape mechanisms. In spite of the short period of the epidemic and the acute nature of the infection significant number of isolates have mutations (up to 30% at some amino acid residue positions) in the receptor-binding domain which is the major neutralization determinant of the SARS CoV Env. However, human antibodies identified from non-immune IgM libraries could efficiently neutralized all SARS CoV isolates with known sequences in contrast to antibodies derived from such libraries against HIV-1.

### Method of Preparing Anti-HIV Vaccine

The following method of preparing a vaccine of the invention is proposed which could be used against any disease, including AIDS and cancer. The preparation of the vaccine is presented with respect to an HIV-specific vaccine, but can be used to prepare vaccines to other diseases, including cancer.

Step 1. Identification of maturational pathway for one or more specific anti-HIV bcrnAb by using a source library. One approach to identify possible maturational pathways is to explore the source library and by using PCR with primers against the CDR3 of the heavy chain to identify a panel of antibodies with almost identical CDR3s; similarly for the light chain with primers against the CDR3. The resulting sequences are analyzed and plausible pathways are reconstituted. Note that multiple pathways are possible; increasing the number of possible pathways increases the immunogenicity of the antigen that leads to maturation of the antibody. Second approach is based on the generation of a library of all possible mutants from the corresponding germline. The library is screened against the HIV Env and the resulting antibodies are arranged according to their increasing affinity. Those antibodies with intermediate number of mutations that show some binding to the Env will be selected and used as intermediate antibodies. Good intermediate antibodies are those which are as close to the germline as possible and still show some (typically small) affinity (avidity) for the epitope of the bcrnAb to be elicited. Yet another approach is to analyze antibodies elicited in HIV infected or immunized humans by using sequential samples and identifying antibodies that bind the Env; an analysis of the sequences of such antibodies especially those corresponding to the same germline as the known bcrnAbs could yield information for the identification of such intermediate antibodies. Similarly samples from non-infected and non-immunized humans could be analyzed for antibodies that are close in sequence to those germline sequences that correspond to the sequences of the known bcrnAbs. Such antibodies are expressed, purified and characterized in terms of their binding ability which typically should be very low even to oligomeric Env.

Step 2. Production of panel of intermediate antibodies. The intermediate antibodies identified as described above will be further characterized and produced in quantities sufficient for the next step.

Step 3. Screening and identification of candidate primary antigens/immunogens. The intermediate antibody(ies) are used for screening of libraries of proteins, antibodies and peptides for identification of candidate antigens that bind with high affinity to the intermediate antibody and with lower affinity to the corresponding germline antibody.

Step 5. Identification of the epitopes of intermediate antigens/immunogens. The epitopes of the intermediate antibodies will be identified by using standard methodologies including competition with already known antibodies with known epitopes, determination of the crystal or NMR structures of the antigen or antigen fragments in complex with the antibody or antibody binding fragments, e.g. scFvs or Fabs. It is likely that these epitopes or portions of them mimic to some extent but not completely the epitope of the bcrnAb that is being elicited.

Step 6. Construction of primary immunogen sufficient to elicit the intermediate antibody. Once the primary antigen is identified and the intermediate antibody epitope characterized standard methods are used for construction of immunogens able to elicit the intermediate antibody. Fragments of the primary antigen that contain the epitope of the intermediate antibody could be used as immunogens. Also fusion proteins of such fragments or the whole antigens with Env-based immunogens (secondary immunogens) could be also used which will obviate the need to use a mixture of primary and secondary immunogens.

Step 7. Obtain suitable HIV antigen as secondary immunogen (e.g., gp41, gp120). Any of the many candidates that can bind with high affinity the desired bcrnAb could serve as secondary immunogen. One such immunogen is the trimeric gp140 from the R2 isolate constructed by C. Broder and G. Quinnan. This construct already elicited bcrnAbs in rabbits and could be successful in combination with the primary immunogen to elicit known bcrnAb against HIV in humans.

Step 8. Administer combination of primary immunogen and secondary immunogen (i.e., the HIV antigen) to elicit bcrnAb. A mixture of the primary and secondary immunogens can be administered as a vaccine immunogen. It is also possible to first administer the primary immunogen and later the secondary immunogen. Another possibility is to make a fusion protein of the primary and secondary immunogen and administer as one entity.

### Nucleic Acids Encoding Immunogens/Antigens

In another aspect, the present invention provides the nucleic acids encoding any of the immunogens of the invention, e.g. the primary or secondary immunogens of the invention. The nucleic acid sequences encoding any of the immunogens of the invention may be obtained by recombinant DNA methods, such as screening reverse transcripts of mRNA, or screening genomic libraries from any HIV-infected cell or HIV isolate. The DNA may also be obtained by synthesizing the DNA from published sequences using commonly available techniques such as solid phase phosphoramidite triester method first described by Beaucage and Caruthers, Tetrahedron Letts. 22:1859-1862 (1981), using an automated synthesizer, as described in Van Devanter et al., Nucleic Acids Res. 12:6159-6168 (1984). Synthesis may be advantageous because unique restriction sites may be introduced at the time of preparing the DNA, thereby facilitating the use of the gene in vectors containing restriction sites not otherwise present in the native source. Furthermore, any desired site modification in the DNA may be introduced by synthesis, without the need to further modify the DNA by mutagenesis.

Purification of oligonucleotides is by either native acrylamide gel electrophoresis, agarose electrophoresis or by anion-exchange HPLC as described in Pearson and Reanier, J. Chrom. 255:137-149 (1983), depending upon the size of the oligonucleotide and other characteristics of the preparation. The sequence of cloned genes and synthetic oligonucleotides can be verified using, e.g., the chain termination method for sequencing double-stranded templates as described by Wallace et al., Gene 16:21-26 (1981).

Processes for producing recombinant immunogens for purification by the methods of the present invention will employ, unless otherwise indicated, conventional molecular biology, microbiology, and recombinant DNA techniques within the skill of the art. Such techniques are explained fully in the literature. See e.g., Maniatis, Fritsch and Sambrook, Molecular Cloning: A Laboratory Manual, 2nd Ed. (1989); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. 1985); Transcription And Translation (B. D. Hames and S. J. Higgins eds. 1984); Animal Cell Culture (R. I. Freshney ed. 1986); Immobilized Cells And Enzymes (IRL Press, 1986); B. Perbal, A Practical Guide To Molecular Cloning (1984); Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

In general, DNA encoding an HIV or primary/secondary immunogen described herein can be obtained by constructing a cDNA library from mRNA recovered from a sample and (1) screening with labeled DNA probes encoding portions of the immunogen of interest in order to detect clones in the cDNA library that contain homologous sequences or (2) amplifying the cDNA using polymerase chain-reaction (PCR) and subcloning and screening with labeled DNA probes. Clones can then be analyzed by restriction enzyme analysis, agarose gel electrophoresis sizing and nucleic acid sequencing so as to identify full-length clones and, if full-length clones are not present in the library, recovering appropriate fragments from the various clones and ligating them at restriction sites common to the clones to assemble a clone encoding a full-length molecule. Any sequences missing from the 5′ end of the cDNA may be obtained by the 3′ extension of the synthetic oligonucleotides complementary to sequences encoding the protein using mRNA as a template (so-called primer extension), or homologous sequences may be supplied from known cDNAs. Polynucleic acid sizes are given in either kilobases (Kb) or base pairs (bp). These sizes are estimates derived from agarose or acrylamide gel electrophoresis, from sequenced nucleic acids, or from published DNA sequences.

Amplification techniques using primers can also be used to isolate HIV envelope glycoproteins from DNA or RNA. Suitable primers are commonly available in the art, which can be synthesized by conventional solid-phase techniques common in the art. Primers can be used, e.g., to amplify either the full length sequence or a probe of one to several hundred nucleotides, which is then used to screen a library for full-length HIV envelope glycoproteins.

Nucleic acids encoding HIV or primary/secondary immunogens of the invention can also be isolated from expression libraries using antibodies as probes.

Immunogen variants or orthologs can be isolated using corresponding nucleic acid probes known in the art to screen libraries under stringent hybridization conditions. Alternatively, expression libraries can be used to clone sequences encoding HIV or primary/secondary immunogens of the invention by detecting expressed proteins immunologically with commercially available antisera or antibodies, or portions thereof, which also recognize and selectively bind to the HIV and primary immunogens.

To make a cDNA library, one should choose a source that is rich in the immunogen of interest, such as the primary R5X4 HIV-1 isolate 89.6 described in Collman, R, et al. “An infectious molecular clone of an unusual macrophage-tropic and highly cytopathic strain of human immunodeficiency virus type 1”, J. Viol., 66, 7517-7521 (1992). The mRNA is then made into cDNA using reverse transcriptase, ligated into a recombinant vector, and transfected into a recombinant host for propagation, screening and cloning. Methods for making and screening cDNA libraries are well known (see, e.g., Gubler and Hoffman, Gene 25:263-269 (1983); Sambrook et al., supra; Ausubel et al., supra).

An alternative method of isolating nucleic acids encoding HIV and primary/secondary immunogens combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify the nucleic acid sequences encoding the glycoproteins directly from mRNA, from cDNA present in genomic libraries or cDNA libraries. Degenerate oligonucleotides can be designed to amplify HIV and intermediate using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of HIV envelope glycoprotein-encoding mRNA in physiological samples, for nucleic acid sequencing, or for other purposes. Genes amplified by the PCR reaction can be purified from agarose gels and cloned into an appropriate vector.

Gene expression of the intermediates of the invention can also be analyzed by techniques known in the art, e.g., reverse transcription and amplification of mRNA, isolation of total RNA or poly A′ RNA, northern blotting, dot blotting, in situ hybridization, RNase protection, high density polynucleotide array technology and the like.

Synthetic oligonucleotides can be used to construct recombinant HIV immunogen genes for use as probes or for expression of protein. This method is performed using a series of overlapping oligonucleotides usually 40-120 by in length, representing both the sense and non-sense (antisense) strands of the gene. These DNA fragments are then annealed, ligated and cloned. Alternatively, amplification techniques can be used with precise primers to amplify a specific gene subsequences for HIV and/or primary immunogens. The specific subsequence is then ligated into a suitable eukaryotic expression vector.

General texts describing additional molecular biological techniques useful herein, including the preparation of antibodies include Berger and Kimmel (Guide to Molecular Cloning Techniques, Methods in Enzymology, Vol. 152, Academic Press, Inc.); Sambrook, et al., (Molecular Cloning: A Laboratory Manual, (Second Edition, Cold Spring Harbor Laboratory Press; Cold Spring Harbor, N.Y.; 1989) Vol. 1-3); Current Protocols in Molecular Biology, (F. M. Ausabel et al. [Eds.], Current Protocols, a joint venture between Green Publishing Associates, Inc. and John Wiley & Sons, Inc. (supplemented through 2000)); Harlow et al., (Monoclonal Antibodies: A Laboratory Manual, Cold Spring Harbor Laboratory Press (1988), Paul [Ed.]); Fundamental Immunology, (Lippincott Williams & Wilkins (1998)); and Harlow, et al., (Using Antibodies: A Laboratory Manual, Cold Spring Harbor Laboratory Press (1998)), all of which are incorporated herein by reference.

### Antibodies and Modifications Thereto

The invention provides methods to generate the antibodies of the invention, e.g., the HIV-specific bcrnAbs, using the primary and secondary immunogens of the invention (e.g., HIV immunogens). The present invention further contemplates additional methods for screening and identifying and obtaining the antibodies of the invention.

In certain examples, a cell line/pseudovirus assay is used as a neutralization assay. Such assays are well-known in the art and easily performed by the skilled practitioner. In Curr Protoc Immunol. 2005 January; Chapter 12: Unit 12.11 18432938, incorporated by reference in its entirety herein, Montefiore et al. describe neutralizing antibody assays as tools for assessing humoral immunity in AIDS virus infection and vaccine development. This reference describes two assays utilizing a genetically engineered cell lines that are susceptible to infection by most strains of HIV-1, SIV, and SHIV. One assay is designed for optimal performance with uncloned viruses produced in either PBMC or CD4(+) T cell lines. A second assay is designed for single-cycle infection with molecularly cloned pseudoviruses produced by transfection in 293T cells. Both assays are performed in a 96-well format and use tat-responsive luciferase reporter gene expression as readout.

Kim et al. (Research and Human Retroviruses. Dec. 10, 2001, 17(18): 1715-1724), incorporated by reference in its entirety herein, describe development of a safe and rapid neutralization assay using murine leukemia virus pseudotyped with HIV Type 1 envelope glycoprotein lacking the cytoplasmic domain.

In other certain examples, a peripheral blood mononuclear cells (PBMC)/primary isolates-based assay can be used as a neutralization assay. Such assays are well-known in the art and easily performed by the skilled practitioner. For example, Montefiore et al. (J. Virol., 03 1997, 2512-2517, Vol 71, No. 3), incorporated by reference in its entirety herein, describe antibody-mediated neutralization of human immunodeficiency virus type 1 (HIV-1) with primary isolates and sera from infected individuals, using human peripheral blood mononuclear cells (PBMC).

For the production of antibodies, various host animals may be immunized by injection with a protein, or a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a cystatin gene product, or an antigenic functional derivative thereof.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein (1975) Nature 256:495-497; and U.S. Pat. No. 4,376,110, the human B-cell hybridoma technique (Kosbor et al. (1983) Immunology Today 4:72; Cole et al. (1983) Proc. Natl. Acad. Sci. USA 80:2026-2030, and the EBV-hybridoma technique (Cole et al. (1985) Monoclonal Antibodies And Cancer Therapy, Alan R. Liss, Inc., pp. 77-96). Such antibodies may be of any immunoglobulin class including IgG, IgM, IgE, IgA, IgD and any subclass thereof.

In addition, techniques developed for the production of “chimeric antibodies” or “humanized antibodies” may be utilized to modify the antibodies of the invention to reduce immunogenicity of non-human antibodies. Morrison et al. (1984) Proc. Natl. Acad. Sci. 81:6851-6855; Neuberger et al. (1984) Nature, 312:604-608; Takeda et al. (1985) Nature, 314:452-454. Such antibodies are generated by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird (1988) Science 242:423-426; Huston et al. (1988) Proc. Natl. Acad. Sci. USA 85:5879-5883; and Ward et al. (1989) Nature 334:544-546) can be adapted to produce single chain antibodies. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments may include but are not limited to: the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed (Huse et al. (1989) Science 246:1275-1281) to allow rapid and easy identification of monoclonal Fab fragments with the desired specificity.

Thus, the method according to an embodiment of the present invention may further comprise screening an antibody library for neutralization antibodies that are specifically immunoreactive against the gp41-based antigens of the invention, e.g., gp41Fc which is the subject of another invention, and which are broadly cross reactive against a wide spectrum of HIV isolates.

The antibodies of the invention can be further modified by methods known in the art. The modifications may be genetic modifications to the nucleic acid encoding the antibodies of the invention or they may be chemical, structural, or physical modifications made directly to an isolated antibody of the invention to impart additional advantageous properties to an antibody of the invention regarding, for example, the level of expression, stability, solubility, epitope affinity, antigen neutralization activity, or penetration characteristics, etc.

In one aspect, the present invention contemplates introducing genetic modifications into one or more CDRs or to the framework sequence of the antibodies of the invention which are identified by methods described herein. Such genetic modifications can confer additional advantageous characteristics, i.e. genetic optimization, of the antibodies identified from library screening, including, for example, enhanced solubility, enhanced affinity, and enhanced stability. Any type of genetic modification is contemplated by the present invention, including, for example, site-directed mutagenesis, random mutagenesis, insertions, deletions, and CDR grafting (i.e. genetic replacement of one CDR for another CDR). All of these techniques are well known to those skilled in the art. See Ausubel et al., Current Protocols in Molecular Biology, John Wiley & Sons, New York, 2000, incorporated herein by reference. Reference to CDR grafting can be made to Nicaise, et al., Protein Science 13:1882-1891, 2004. The effect of any genetic modification can be tested or screened without undue experimentation using any of the methods described herein or other methods already known to one of ordinary skill in the art. For example, affinity of an antibody to a target antigen can be assessed using the herein described BIA procedure.

In another aspect, other modifications contemplated by the present invention relate to chemical modifications of the antibodies of the invention to confer additional advantageous features, such as enhanced stability and/or solubility and/or half-life.

In one particular aspect, the antibodies of the present invention can be PEGylated, or coupled to polymers of similar structure, function and purpose (“PEG or PEG-like polymers”), to confer enhanced stability and half-life. PEGylation can provide increased half-life and resistance to degradation without a loss in activity (e.g. binding affinity) relative to non-PEGylated antibody polypeptides. The skilled artisan will appreciate, however, that PEGylation may not be advantageous with respect to some targets, in particular, those epitopes which are sterically-obstructed. Thus, in cases where the inventive antibodies targets a size-restricted epitope, the antibody should be minimally PEGylated so as not to negatively impact the accessibility of the antibody to the size-restricted antigen. The skilled artisan will appreciate that this general principle should be applied to any modifications made to the antibodies of the invention.

Any method known in the art to couple the antibodies of the invention to PEG or PEG-like polymers is contemplated by the present invention. PEG or PEG-like moieties which can be utilized in the invention can be synthetic or naturally occurring and include, but are not limited to, straight or branched chain polyalkylene, polyalkenylene or polyoxyalkylene polymers, or a branched or unbranched polysaccharide, such as a homo- or heteropolysaccharide. Preferred examples of synthetic polymers which can be used in the invention include straight or branched chain poly(ethylene glycol) (PEG), poly(propylene glycol), or poly(vinyl alcohol) and derivatives or substituted forms thereof. Substituted polymers for linkage to the antibodies of the invention can also particularly include substituted PEG, including methoxy(polyethylene glycol). Naturally occurring polymer moieties which can be used in addition to or in place of PEG include, for example, lactose, amylose, dextran, or glycogen, as well as derivatives thereof which would be recognized by persons skilled in the art.

PEGylation of the antibodies of the invention may be accomplished by any number of means (see for example Kozlowski-A & Harris-J M (2001) Journal of Controlled Release 72:217). PEG may be attached to an antibody construct either directly or by an intervening linker. Linkerless systems for attaching polyethylene glycol to proteins is described in Delgado et al., (1992), Crit. Rev. Thera. Drug Carrier Sys. 9:249-304 Francis et al., (1998), Intern. J. Hematol. 68:1-18; U.S. Pat. No. 4,002,531; U.S. Pat. No. 5,349,052; WO 95/06058; and WO 98/32466, the disclosures each of which are incorporated herein by reference. The first step in the attachment of PEG or other polymer moieties to the antibody construct of the invention typically is the substitution of the hydroxyl end-groups of the PEG polymer by electrophile-containing functional groups. Particularly, PEG polymers are attached to either cysteine or lysine residues present in the antibody construct monomers or multimers. The cysteine and lysine residues can be naturally occurring, or can be engineered into the antibody molecule.

One system for attaching polyethylene glycol directly to amino acid residues of proteins without an intervening linker employs tresylated MPEG, which is produced by the modification of monomethoxy polyethylene glycol (MPEG) using tresylchloride. Following reaction of amino acid residues with tresylated MPEG, polyethylene glycol is directly attached to the amine groups. Thus, the invention includes protein-polyethyleneglycol conjugates produced by reacting proteins of the invention with a polyethylene glycol molecule having a 2,2,2-trifluoreothane sulphonyl group.

Polyethylene glycol can also be attached to proteins using a number of different intervening linkers. For example, U.S. Pat. No. 5,612,460 discloses urethane linkers for connecting polyethylene glycol to proteins. Protein-polyethylene glycol conjugates wherein the polyethylene glycol is attached to the protein by a linker can also be produced by reaction of proteins with compounds such as MPEG-succinimidylsuccinate, MPEG activated with 1,1′-carbonyldiimidazole, MPEG-2,4,5-trichloropenylcarbonate, MPEG-p-nitrophenolcarbonate, and various MPEG-succinate derivatives. A number of additional polyethylene glycol derivatives and reaction chemistries for attaching polyethylene glycol to proteins are described in WO 98/32466, the entire disclosure of which is incorporated herein by reference.

Other derivatized forms of polymer molecules include, for example, derivatives which have additional moieties or reactive groups present therein to permit interaction with amino acid residues of the antibodies described herein. Such derivatives include N-hydroxylsuccinimide (NHS) active esters, succinimidyl propionate polymers, and sulfhydryl-selective reactive agents such as maleimide, vinyl sulfone, and thiol. The reactive group (e.g., MAL, NHS, SPA, VS, or Thiol) may be attached directly to the PEG polymer or may be attached to PEG via a linker molecule.

The size of polymers useful in the invention can be in the range of 500 Da to 60 kDa, for example, between 1000 Da and 60 kDa, 10 kDa and 60 kDa, 20 kDa and 60 kDa, 30 kDa and 60 kDa, 40 kDa and 60 kDa, and up to between 50 kDa and 60 kDa. The polymers used in the invention, particularly PEG, can be straight chain polymers or may possess a branched conformation.

The present invention also contemplates the coupling of adduct molecules, which can be various polypeptides or fragments thereof which occur naturally in vivo and which resist degradation or removal by endogenous mechanisms. Molecules which increase half life may be selected from the following: (a) proteins from the extracellular matrix, eg. collagen, laminin, integrin and fibronectin; (b) proteins found in blood, e.g., serum albumin, fibrinogen A, fibrinogen B, serum amyloid protein A, heptaglobin, protein, ubiquitin, uteroglobulin, B-2 microglobulin, plasminogen, lysozyme, cystatin C, alpha-1-antitrypsin and pancreatic kypsin inhibitor; (c) immune serum proteins, e.g. IgE, IgG, IgM and their fragments e.g. Fc; (d) transport proteins, e.g. retinol binding protein; (e) defensins, e.g. beta-defensin 1, neutrophil defensins 1, 2 and 3; (f) proteins found at the blood brain barrier or in neural tissues, e.g. melanocortin receptor, myelin, ascorbate transporter; (g) transferrin receptor specific ligand-neuropharmaceutical agent fusion proteins, brain capillary endothelial cell receptor, transferrin, transferrin receptor, insulin, insulin-like growth factor 1 (IGF 1) receptor, insulin-like growth factor 2 (IGF 2) receptor, insulin receptor; (h) proteins localised to the kidney, e.g. polycystin, type IV collagen, organic anion transporter Kl, Heymann's antigen; (i) proteins localized to the liver, e.g. alcohol dehydrogenase, G250; (j) blood coagulation factor X; (k) α-1 antitrypsin; (1) HNF 1α.; (m) proteins localised to the lung, e.g. secretory component (binds IgA); (n) proteins localised to the heart, eg. HSP 27; (O) proteins localised to the skin, eg, keratin; (p) bone specific proteins, such as bone morphogenic proteins (BMPs) e.g. BMP-2, -4, -5, -6, -7 (also referred to as osteogenic protein (OP-1) and -8 (OP-2); (q) tumour specific proteins, eg. human trophoblast antigen, herceptin receptor, oestrogen receptor, cathepsins eg cathepsin B (found in liver and spleen); (r) disease-specific proteins, eg. antigens expressed only on activated T-cells: including LAG-3 (lymphocyte activation gene); osteoprotegerin ligand (OPGL) see Kong Y Y et al Nature (1999) 402, 304-309; OX40 (a member of the TNF receptor family, expressed on activated T cells and the only costimulatory T cell molecule known to be specifically up-regulated in human T cell leukaemia virus type-I (HTLV-I)-producing cells—see Pankow R et al J. Immunol. (2000) Jul. 1; 165(1):263-70; metalloproteases (associated with arthritis/cancers), including CG6512 Drosophila, human paraplegin, human FtsH, human AFG3L2, murine ftsH; angiogenic growth factors, including acidic fibroblast growth factor (FGF-1), basic fibroblast growth factor (FGF-2), Vascular endothelial growth factor/vascular permeability factor (VEGF/VPF), transforming growth factor-α (TGF-α), tumor necrosis factor-alpha (TNF-α), angiogenin, interleukin-3 (IL-3), interleukin-8 (IL-8), platelet derived endothelial growth factor (PD-ECGF), placental growth factor (P1GF), midkine platelet-derived growth factor-BB (PDGF), fractalkine; (s) stress proteins (heat shock proteins); and (t) proteins involved in Fc transport.

In another aspect, the antibodies of the invention may be multimerized, as for example, hetero- or homodimers, hetero- or homotrimers, hetero- or homotetramers, or higher order hetero- or homomultimers. Multimerisation can increase the strength of antigen binding, wherein the strength of binding is related to the sum of the binding affinities of the multiple binding sites. The antibodies can be multimerized in another aspect by binding to an additional one, two, three or more polypeptide which function to stabilize the dAb against degradation. Such polypeptides may include common blood proteins, such as, albumin, or fragments thereof.

In yet another aspect, modifications relating to enhancing or modifying antibody activity are contemplated by the present invention. For example, it may be desirable to modify the antibody of the invention with respect to effector function, so as to enhance the effectiveness of the antibody in treating a condition, infection or disorder. For example cysteine residue(s) may be introduced in the antibody polypeptide, thereby allowing interchain disulfide bond formation in a multimerized form of the inventive antibodies. The homodimeric or heterodimeric (or multimeric) antibodies may include combinations of the same antibody polypeptide chains or different antibody polypeptide chains, such that more than one epitope is targeted at a time by the same construct. Such epitopes can be proximally located in the target (e.g. on the HIV target) such that the binding of one epitope facilitates the binding of the multmeric antibody of the invention to the second or more epitopes. The epitopes targeted by multimeric antibodies can also be distally situated.

The invention also contemplates modifying the antibodies of the invention to form immunoconjugates comprising the antibodies of the invention conjugated to cytotoxic agents, such as a chemotherapeutic agents, toxin (e.g., an enzymatically active toxin of bacterial, fungal, plant or animal origin, or fragments thereof), radioactive isotopes (i.e., a radioconjugate), or antiviral compounds (e.g. anti-HIV compounds).

The term “cytotoxic agent” as used herein refers to a substance that inhibits or prevents the function of cells and/or causes destruction of cells. The term can include radioactive isotopes (e.g., I₁₃₁, I₁₂₅, Y₉₀ and Re₁₈₆), chemotherapeutic agents, and toxins such as enzymatically active toxins of bacterial, fungal, plant or animal origin, or fragments thereof.

A “chemotherapeutic agent” is a type of cytotoxic agent useful in the treatment of cancer. Examples of chemotherapeutic agents include Adriamycin, Doxorubicin, 5-Fluorouracil, Cytosine arabinoside (“Ara-C”), Cyclophosphamide, Thiotepa, Taxotere (docetaxel), Busulfan, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Caminomycin, Aminopterin, Dactinomycin, Mitomycins, Esperamicins, Melphalan and other related nitrogen mustards.

The invention also contemplates immunoconjugation with enzymatically active toxins or fragments thereof. Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes.

Where the inventive antibodies are intended to target HIV infections that might also involve infection by other viruses, bacteria or other pathogens, the invention also contemplates immunoconjugation of the antibodies with anti-viral, anti-bacterial or other chemicals and/or compounds that might improve or increase the effectiveness of the antibodies of the invention against intended targets, such as, for example, HIV.

For example, the inventive antibodies can be immunoconjugated, or in the alternative, co-administered with, an antibacterial compound, such as, for example, a macrolide (e.g., tobramycin (TOBI®)), a cephalosporin (e.g., cephalexin (KEFLEX®), cephradine (VELOSEF®), cefuroxime (CEFTIN®), cefprozil (CEFZIL®), cefaclor (CECLOR®), cefixime (SUPRAX®) or cefadroxil (DURICEF®), a clarithromycin (e.g., clarithromycin (BIAXIN®)), an erythromycin (e.g., erythromycin (EMYCIN®)), a penicillin (e.g., penicillin V (V-CILLIN K® or PEN VEE K®)) or a quinolone (e.g., ofloxacin (FLOXINO), ciprofloxacin (CIPRO®) or norfloxacin (NOROXIN®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitro furans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

In another example, the inventive antibodies can be immunoconjugated, or in the alternative, co-administered with, an antiviral compound, such as, for example, a zidovudine, acyclovir, gangcyclovir, vidarabine, idoxuridine, trifluridine, and ribavirin, as well as foscarnet, amantadine, rimantadine, saquinavir, indinavir, amprenavir, lopinavir, ritonavir, adefovir, clevadine, entecavir, and pleconaril.

Methods for modifying the antibodies of the invention with the various cytoxic agents, chemotherapeutic agents, toxins, antibacterial compounds, and antiviral compounds, etc. mentioned above are well known in the art. For example, immunoconjugates of the antibody and cytotoxic agents can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

The antibodies can also be modified with useful detectable agents, such as, for example, fluorescent compounds. Exemplary fluorescent detectable agents include fluorescein, fluorescein isothiocyanate, rhodamine, 5-dimethylamine-1-napthalenesulfonyl chloride, phycoerythrin and the like. The antibody construct may also be derivatized with detectable enzymes such as alkaline phosphatase, horseradish peroxidase, glucose oxidase and the like. When the antibody construct is derivatized with a detectable enzyme, it is detected by adding additional reagents that the enzyme uses to produce a detectable reaction product. The antibody construct may also be derivatized with biotin, and detected through indirect measurement of avidin or streptavidin binding.

The skilled artisan will appreciate it may be advantageous to couple any of the aforementioned molecular entities to the antibodies of the invention through flexible linkers, such as flexible polypeptide chains. Such linkers may be required to avoid a loss in activity of the antibodies, or to avoid sterically restricting the antibodies such that they lose their effectiveness in binding to cognate epitopes, in particular, those epitopes which themselves may be sterically restricted. The linkers can be the same or different as the linkers described herein elsewhere which are used to fuse the gp41 subunit (or fragment or derivative thereof) with the Fc receptor ligand.

Another type of covalent modification contemplated by the present invention involves chemically or enzymatically coupling glycosides to the antibodies of the invention. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published Sep. 11, 1987, and in Aplin and Wriston, CRC Crt. Rev. Biochem., pp. 259-306 (1981).

Removal of any carbohydrate moieties present on the antibodies of the invention may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Hakimuddin, et al. Arch. Biochem. Biophys. 259:52 (1987) and by Edge et al. Anal. Biochem., 118:131 (1981). Enzymatic cleavage of carbohydrate moieties on antibodies can be achieved by the use of a variety of endo- and exo-glycosidases as described by Thotakura et al. Meth. Enzymol. 138:350 (1987).

Analytical/Preparative Methods for Antigens and/or Antibodies of Invention

Once an immunogen or antibody in accordance with the invention is identified or obtained, for example, by any of the methods herein described, it may be preferable to carry out further steps to characterize and/or purify and/or modify the antigen or antibody. For example, it may be desirable to prepare a purified, high-titer composition of the desirable antibody or to test the immunoreactivity of the identified antibody. The present invention contemplates any known and suitable methods for characterizing, purifying, or assaying the antigens and/or antibodies of the present invention and it is expected the any person of ordinary skill in the art to which the invention pertains will have the requisite level of technical know-how and resources, e.g. technical manuals or treatises, to accomplish any further characterization, purification and/or assaying of the antigens and/or antibodies of the invention without undue experimentation.

For example, any useful means to describe the strength of binding (or affinity) between a antibody of the invention and an antigen of the invention (e.g., gp41Fc) can be used. For example, the dissociation constant, K_{d} (K_{d}=k2/k1=[antibody][antigen]/[antibody-antigen complex]) can be determined by standard kinetic analyses that are known in the art. It will be appreciated by those of ordinary skill in the art that the dissociation constant indicates the strength of binding between an antibody and an antigen in terms of how easy it is to separate the complex. If a high concentration of antibody and antigen are required to form the complex, the strength or affinity of binding is low, resulting in a higher IQ. It follows that the smaller the K_{d} (as expressed in concentration units, e.g. molar or nanomolar), the stronger the binding.

Affinity can be assessed and/or measured by a variety of known techniques and immunoassays, including, for example, enzyme-linked immunospecific assay (ELISA), Bimolecular Interaction Analysis (BIA) (e.g., Sjolander and Urbaniczky, Anal. Chem. 63:2338-2345, 1991; Szabo, et al., Curr. Opin. Struct. Biol. 5:699-705, 1995, each incorporated herein by reference), and fluorescence-activated cell sorting (FACS) for quantification of antibody binding to cells that express antigen. BIA is a technology for analyzing biospecific interactions in real time, without labeling any of the interactants (e.g., BIACORE™). BIAcore is based on determining changes in the optical phenomenon surface plasmon resonance (SPR) in real-time reactions between biological molecules, such as, an antibody of the invention and an antigen of interest, e.g. CD4i. References relating to BIAcore technology can be further found in U.S. Published Application Nos: 2006/0223113, 2006/0134800, 2006/0094060, 2006/0072115, 2006/0019313, 2006/0014232, and 2005/0199076, each of which are incorporated herein in their entireties by reference.

The antigens and antibodies of the invention may be assayed for immunospecific binding by any suitable method known in the art. Assays involving an antibody and an antigen are known as “immunoassays,” which can be employed in the present invention to characterize both the antibodies and the antigens of the invention. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety) and can be performed without undue experimentation.

Western blot analysis generally comprises preparing protein samples, electrophoresis of the protein samples in a polyacrylamide gel (e.g., 8% 20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer; blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ₃₂P or ₁₂₅I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, 1994, Current Protocols in Molecular Biology, Vol. 1, John Wiley & Sons, Inc., New York at 10.8.1, which is incorporated herein by reference.

ELISAs typically comprise preparing antigen (e.g., gp140), coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1, which is incorporated herein by reference.

Any suitable method for purifying antigens and/or antibodies of the invention is contemplated herein. For example, chromatographic methods, such as, for example, immuno-affinity chromatography (immobilized ligand to bind and trap antibody of interest), affinity chromatography, protein precipitation, ion exchange chromatography, hydrophobic interaction chromatography, size-exclusion chromatography, as well as electrophoresis, can be found described in the technical literature, for example, in Methods in Enzymology, Volume 182, Guide to Protein Purification, Eds. J. Abelson, M. Simon, Academic Press, 1^{st} Edition, 1990, which is incorporated herein by reference. Thus, suitable materials for performing such purification steps, such as chromatographic steps, are known to those skilled in the art. Such methods are suitable for purification of any of the antibodies, antigens or any fragments thereof that are in accordance with the invention as described herein.

Certain embodiments may require the purification or isolation of expressed antigens or antibodies or fragments thereof from a host cell or a portion thereof. Conventional procedures for isolating recombinant proteins from transformed host cells are contemplated by the present invention. Such methods include, for example, isolation of the protein or fragments of interest by initial extraction from cell pellets or from cell culture medium, followed by salting-out, and one or more chromatography steps, including aqueous ion exchange chromatography, size exclusion chromatography steps, high performance liquid chromatography (HPLC), and affinity chromatography may be used to isolate the recombinant protein or fragment. Guidance in the procedures for protein purification can be found in the technical literature, including, for example, Methods in Enzymology, Volume 182, Guide to Protein Purification, Eds. J. Abelson, M. Simon, Academic Press, 1^{st} Edition, 1990, which is already incorporated by reference.

### Methods of Use

The present invention provides pharmaceutical compositions comprising a therapeutically effective amount of the antigens and/or antibodies of the invention, together with a pharmaceutically acceptable carrier.

In one aspect, the present invention provides a method for vaccinating against an HIV infection by administering a therapeutically effective amount of the vaccine (e.g., the primary and secondary immunogens of the invention) of the invention, together with a pharmaceutically acceptable carrier or diluent. Administration can occur before or after HIV infection.

In another aspect, the present invention provides a method for treating an HIV infection by administering a therapeutically effective amount of an antibody and/or immunogen of the invention, together with a pharmaceutically acceptable carrier or diluent. Administration can occur before or after HIV infection.

Some terms relating to the use of the antigens and/or antibodies of this invention are defined as follows.

The term “treatment” includes any process, action, application, therapy, or the like, wherein a subject (or patient), including a human being, is provided medical aid with the object of improving the subject's condition, directly or indirectly, or slowing the progression of a condition or disorder in the subject, or ameliorating at least one symptom of the disease or disorder under treatment.

The term “combination therapy” or “co-therapy” means the administration of two or more therapeutic agents (e.g., the primary and secondary immunogens) to treat a disease, condition, and/or disorder. Such administration encompasses co-administration of two or more therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner. The order of administration of two or more sequentially co-administered therapeutic agents is not limited.

The phrase “therapeutically effective amount” means the amount of each agent administered that will achieve the goal of improvement in a disease, condition, and/or disorder severity, and/or symptom thereof, while avoiding or minimizing adverse side effects associated with the given therapeutic treatment.

The term “pharmaceutically acceptable” means that the subject item is appropriate for use in a pharmaceutical product.

The antibodies and immunogens of this invention are expected to be valuable as therapeutic agents, e.g. anti-HIV antibody based therapies, due to their high degree of cross-reactivity against HIV isolates and their ability to neutralize a wide spectrum of HIV types. Accordingly, an embodiment of this invention includes a method of treating and/or preventing a particular condition (e.g. HIV infection) in a patient which comprises administering to said patient a composition containing an amount of an antibody of the invention that is effective in treating the target condition, e.g., HIV infection.

The antigens of this invention are expected to be valuable as vaccine immunogens due to their enhanced immunogenicity, enhanced stability and half-life, and their ability to elicit effective neutralizing antibodies that are broadly cross-reactive against a spectrum of HIV isolates and do not react with self-antigens (unlike antibodies elicited by known gp41-based antigens). Accordingly, an embodiment of this invention includes a method of vaccinating against HIV infections in a subject comprising administering to said subject a pharmaceutical composition containing an amount of an antigen of the invention that is effective in immunizing (at least partially) against HIV infection.

The antigens and/or antibodies of the present invention may be administered alone or in combination with one or more additional therapeutic agents. Combination therapy includes administration of a single pharmaceutical dosage formulation which contains an antibody of the present invention and one or more additional therapeutic agents, as well as administration of the antibody of the present invention and each additional therapeutic agents in its own separate pharmaceutical dosage formulation. For example, an antibody of the present invention and a therapeutic agent may be administered to the patient together in a single oral dosage composition or each agent may be administered in separate oral dosage formulations.

Where separate dosage formulations are used, the antibody of the present invention and one or more additional therapeutic agents may be administered at essentially the same time (e.g., concurrently) or at separately staggered times (e.g., sequentially). The order of administration of the agents is not limited.

For example, in one aspect, co-administration of an antibody or antibody fragment of the invention together with one or more anti-HIV agents to potentiate the effect of either the antibody or the anti-HIV agent(s) or both is contemplated for use in treating HIV infections. Examples of anti-HIV agents include, but are not limited to AGENERASE (ampreavir), APTIVUS (tipranavir), ATRIPLA, COMBIVIR, RETROVIR, EPIVIR, CRIXIVAN (indinavir), EMTRIVA (emtricitabine), EPZICOM, FORTOVASE (saquinavir), FUZEON (enfuvirtide), HIVID (ddc/zalcitabine), INTELENCE (Etravirine), ISENTRESS (raltegravir), INVIRASE (saquinavir), KAETRA (lopinavir), LEXIVA (Fosamprenavir), NORVIR (ritonavir), PREZISTA (darunavir), RESCRTIPTOR (delavirdine), RETROVIR (AZT), REYATAZ (atazanavir), SUSTIVA (efavirenz), TRIZIVIR, VIDEX (ddI/didanosine), VIRACEPT (nelfinavir), VIRAMUNE (nevirapine), VIREAD (tenofovir disoproxil fumarate), ZERIT (d4t/stavudine) and ZIAGEN (abacavir).

The one or more anti-cancer agents can include any known and suitable compound in the art, such as, for example, chemoagents, other immunotherapeutics, cancer vaccines, anti-angiogenic agents, cytokines, hormone therapies, gene therapies, and radiotherapies. A chemoagent (or “anti-cancer agent” or “anti-tumor agent” or “cancer therapeutic”) refers to any molecule or compound that assists in the treatment of a cancer. Examples of chemoagents contemplated by the present invention include, but are not limited to, cytosine arabinoside, taxoids (e.g., paclitaxel, docetaxel), anti-tubulin agents (e.g., paclitaxel, docetaxel, epothilone B, or its analogues), macrolides (e.g., rhizoxin) cisplatin, carboplatin, adriamycin, tenoposide, mitozantron, discodermolide, eleutherobine, 2-chlorodeoxyadenosine, alkylating agents (e.g., cyclophosphamide, mechlorethamine, thioepa, chlorambucil, melphalan, carmustine (BSNU), lomustine (CCNU), cyclothosphamide, busulfan, dibromomannitol, streptozotocin, mitomycin C, and cis-dichlorodiamine platinum (II) (DDP) cisplatin, thio-tepa), antibiotics (e.g., dactinomycin (formerly actinomycin), bleomycin, mithramycin, anthramycin), antimetabolites (e.g., methotrexate, 6-mercaptopurine, 6-thioguanine, cytarabine, flavopiridol, 5-fluorouracil, fludarabine, gemcitabine, dacarbazine, temozolamide), asparaginase, Bacillus Calmette and Guerin, diphtheria toxin, hexamethylmelamine, hydroxyurea, LYSODREN, nucleoside analogues, plant alkaloids (e.g., Taxol, paclitaxel, camptothecin, topotecan, irinotecan (CAMPTOSAR, CPT-11), vincristine, vinca alkyloids such as vinblastine), podophyllotoxin (including derivatives such as epipodophyllotoxin, VP-16 (etoposide), VM-26 (teniposide)), cytochalasin B, colchine, gramicidin D, ethidium bromide, emetine, mitomycin, procarbazine, mechlorethamine, anthracyclines (e.g., daunorubicin (formerly daunomycin), doxorubicin, doxorubicin liposomal), dihydroxyanthracindione, mitoxantrone, mithramycin, actinomycin D, procaine, tetracaine, lidocaine, propranolol, puromycin, anti-mitotic agents, abrin, ricin A, pseudomonas exotoxin, nerve growth factor, platelet derived growth factor, tissue plasminogen activator, aldesleukin, allutamine, anastrozle, bicalutamide, biaomycin, busulfan, capecitabine, carboplain, chlorabusil, cladribine, cylarabine, daclinomycin, estramusine, floxuridhe, gamcitabine, gosereine, idarubicin, itosfamide, lauprolide acetate, levamisole, lomusline, mechlorethamine, magestrol, acetate, mercaptopurino, mesna, mitolanc, pegaspergase, pentoslatin, picamycin, riuxlmab, campath-1, straplozocin, thioguanine, tretinoin, vinorelbine, or any fragments, family members, or derivatives thereof, including pharmaceutically acceptable salts thereof. Compositions comprising one or more chemoagents (e.g., FLAG, CHOP) are also contemplated by the present invention. FLAG comprises fludarabine, cytosine arabinoside (Ara-C) and G-CSF. CHOP comprises cyclophosphamide, vincristine, doxorubicin, and prednisone.

The chemoagent can be an anti-angiogenic agent, such as, for example, angiostatin, bevacizumab (Avastin®), sorafenib (Nexavar®), baculostatin, canstatin, maspin, anti-VEGF antibodies or peptides, anti-placental growth factor antibodies or peptides, anti-Flk-1 antibodies, anti-Flt-1 antibodies or peptides, laminin peptides, fibronectin peptides, plasminogen activator inhibitors, tissue metalloproteinase inhibitors, interferons, interleukin 12, IP-10, Gro-β, thrombospondin, 2-methoxyoestradiol, proliferin-related protein, carboxiamidotriazole, CM101, Marimastat, pentosan polysulphate, angiopoietin 2, interferon-alpha, herbimycin A, PNU145156E, 16K prolactin fragment, Linomide, thalidomide, pentoxifylline, genistein, TNP-470, endostatin, paclitaxel, accutin, cidofovir, vincristine, bleomycin, AGM-1470, platelet factor 4 or minocycline. Without being bound by theory, the co-administration of an anti-angiogenic agent advantageously may lead to the increase in MN expression in a tumor, thereby making the tumor more susceptible to the antibodies and antibody conjugates of the invention.

In one aspect, said chemoagent is gemcitabine at a dose ranging from 100 to 1000 mg/m²/cycle. In one embodiment, said chemoagent is dacarbazine at a dose ranging from 200 to 4000 mg/m² cycle. In another aspect, said dose ranges from 700 to 1000 mg/m²/cycle. In yet another aspect, said chemoagent is fludarabine at a dose ranging from 25 to 50 mg/m²/cycle. In another aspect, said chemoagent is cytosine arabinoside (Ara-C) at a dose ranging from 200 to 2000 mg/m²/cycle. In still another aspect, said chemoagent is docetaxel at a dose ranging from 1.5 to 7.5 mg/kg/cycle. In yet another aspect, said chemoagent is paclitaxel at a dose ranging from 5 to 15 mg/kg/cycle. In a further aspect, said chemoagent is cisplatin at a dose ranging from 5 to 20 mg/kg/cycle. In a still further aspect, said chemoagent is 5-fluorouracil at a dose ranging from 5 to 20 mg/kg/cycle. In another aspect, said chemo agent is doxorubicin at a dose ranging from 2 to 8 mg/kg/cycle. In yet a further aspect, said chemoagent is epipodophyllotoxin at a dose ranging from 40 to 160 mg/kg/cycle. In yet another aspect, said chemoagent is cyclophosphamide at a dose ranging from 50 to 200 mg/kg/cycle. In a further aspect, said chemoagent is irinotecan at a dose ranging from 50 to 150 mg/m²/cycle. In a still further aspect, said chemoagent is vinblastine at a dose ranging from 3.7 to 18.5 mg/m²/cycle. In another aspect, said chemoagent is vincristine at a dose ranging from 0.7 to 2 mg/m²/cycle. In one aspect, said chemoagent is methotrexate at a dose ranging from 3.3 to 1000 mg/m²/cycle.

In another aspect, the antigens and/or antibodies of the present invention are administered in combination with one or more immunotherapeutic agents, such as antibodies or immunomodulators, which include, but are not limited to, HERCEPTIN®, RETUXAN®, OvaRex, Panorex, BEC2, IMC-C225, Vitaxin, Campath I/H, Smart MI95, LymphoCide, Smart I D10, and Oncolym, rituxan, rituximab, gemtuzumab, or trastuzumab.

The invention also contemplates administering the antigens and/or antibodies of the present invention with one or more anti-angiogenic agents, which include, but are not limited to, angiostatin, thalidomide, kringle 5, endostatin, Serpin (Serine Protease Inhibitor) anti-thrombin, 29 kDa N-terminal and a 40 kDa C-terminal proteolytic fragments of fibronectin, 16 kDa proteolytic fragment of prolactin, 7.8 kDa proteolytic fragment of platelet factor-4, a β-amino acid peptide corresponding to a fragment of platelet factor-4 (Maione et al., 1990, Cancer Res. 51:2077), a 14-amino acid peptide corresponding to a fragment of collagen I (Tolma et al., 1993, J. Cell Biol. 122:497), a 19 amino acid peptide corresponding to a fragment of Thrombospondin I (Tolsma et al., 1993, J. Cell Biol. 122:497), a 20-amino acid peptide corresponding to a fragment of SPARC (Sage et al., 1995, J. Cell. Biochem. 57:1329-), or any fragments, family members, or derivatives thereof, including pharmaceutically acceptable salts thereof.

Other peptides that inhibit angiogenesis and correspond to fragments of laminin, fibronectin, procollagen, and EGF have also been described (See the review by Cao, 1998, Prog. Mol. Subcell. Biol. 20:161). Monoclonal antibodies and cyclic pentapeptides, which block certain integrins that bind RGD proteins (i.e., possess the peptide motif Arg-Gly-Asp), have been demonstrated to have anti-vascularization activities (Brooks et al., 1994, Science 264:569; Hammes et al., 1996, Nature Medicine 2:529). Moreover, inhibition of the urokinase plasminogen activator receptor by antagonists inhibits angiogenesis, tumor growth and metastasis (Min et al., 1996, Cancer Res. 56:2428-33; Crowley et al., 1993, Proc Natl Acad. Sci. USA 90:5021). Use of such anti-angiogenic agents is also contemplated by the present invention.

The antigens and/or antibodies of the present invention can also be administered in combination with one or more cytokines, which includes, but is not limited to, lymphokines, tumor necrosis factors, tumor necrosis factor-like cytokines, lymphotoxin-α, lymphotoxin-β, interferon-β, macrophage inflammatory proteins, granulocyte monocyte colony stimulating factor, interleukins (including, but not limited to, interleukin-1, interleukin-2, interleukin-6, interleukin-12, interleukin-15, interleukin-18), OX40, CD27, CD30, CD40 or CD137 ligands, Fas-Pas ligand, 4-1BBL, endothelial monocyte activating protein or any fragments, family members, or derivatives thereof, including pharmaceutically acceptable salts thereof.

The antigens and/or antibodies of the present invention can also be administered in combination with a cancer vaccine, examples of which include, but are not limited to, autologous cells or tissues, non-autologous cells or tissues, carcinoembryonic antigen, alpha-fetoprotein, human chorionic gonadotropin, BCG live vaccine, melanocyte lineage proteins (e.g., gp100, MART-1/MelanA, TRP-1 (gp75), tyrosinase, widely shared tumor-associated, including tumor-specific, antigens (e.g., BAGE, GAGE-1, GAGE-2, MAGE-1, MAGE-3, N-acetylglucosaminyltransferase-V, p15), mutated antigens that are tumor-associated (β-catenin, MUM-1, CDK4), nonmelanoma antigens (e.g., HER-2/neu (breast and ovarian carcinoma), human papillomavirus-E6, E7 (cervical carcinoma), MUC-1 (breast, ovarian and pancreatic carcinoma). For human tumor antigens recognized by T-cells, see generally Robbins and Kawakami, 1996, Curr. Opin. Immunol. 8:628. Cancer vaccines may or may not be purified preparations.

In yet another embodiment, the antigens and/or antibodies of the present invention are used in association with a hormonal treatment. Hormonal therapeutic treatments comprise hormonal agonists, hormonal antagonists (e.g., flutamide, tamoxifen, leuprolide acetate (LUPRON), LH-RH antagonists), inhibitors of hormone biosynthesis and processing, and steroids (e.g., dexamethasone, retinoids, betamethasone, cortisol, cortisone, prednisone, dehydrotestosterone, glucocorticoids, mineralocorticoids, estrogen, testosterone, progestins), antigestagens (e.g., mifepristone, onapristone), and antiandrogens (e.g., cyproterone acetate).

The antigens and/or antibodies described herein may be provided in a pharmaceutical composition comprising a pharmaceutically acceptable carrier. The pharmaceutically acceptable carrier may be non-pyrogenic. The compositions may be administered alone or in combination with at least one other agent, such as stabilizing compound, which may be administered in any sterile, biocompatible pharmaceutical carrier including, but not limited to, saline, buffered saline, dextrose, and water. A variety of aqueous carriers may be employed including, but not limited to saline, glycine, or the like. These solutions are sterile and generally free of particulate matter. These solutions may be sterilized by conventional, well-known sterilization techniques (e.g., filtration).

Generally, the phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the antibody compositions of the invention.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The compositions may contain pharmaceutically acceptable auxiliary substances as required to approximate physiological conditions such as pH adjusting and buffering agents, and the like. The concentration of the antibody of the invention in such pharmaceutical formulation may vary widely, and may be selected primarily based on fluid volumes, viscosities, etc., according to the particular mode of administration selected. If desired, more than one type of antibody may be included in a pharmaceutical composition (e.g., an antibody with different K_{d} for MN binding).

The compositions may be administered to a patient alone, or in combination with other agents, drugs or hormones. In addition to the active ingredients, these pharmaceutical compositions may contain suitable pharmaceutically acceptable carriers comprising excipients and auxiliaries that facilitate processing of the active compounds into preparations which may be used pharmaceutically. Pharmaceutical compositions of the invention may be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means.

The compositions of the invention additionally contemplate suitable immunocarriers, such as, proteins, polypeptides or peptides such as albumin, hemocyanin, thyroglobulin and derivatives thereof, particularly bovine serum albumin (BSA) and keyhole limpet hemocyanin (KLH), polysaccharides, carbohydrates, polymers, and solid phases. Other protein-derived or non-protein derived substances are known to those skilled in the art.

Formulations suitable for parenteral, subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000). Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The determination of a therapeutically effective dose is well within the capability of those skilled in the art. A therapeutically effective dose refers to the amount of an antibody that may be used to effectively treat a disease (e.g., cancer) compared with the efficacy that is evident in the absence of the therapeutically effective dose.

The therapeutically effective dose may be estimated initially in animal models (e.g., rats, mice, rabbits, dogs, or pigs). The animal model may also be used to determine the appropriate concentration range and route of administration. Such information may then be used to determine useful doses and routes for administration in humans.

Therapeutic efficacy and toxicity (e.g., ED₅₀—the dose therapeutically effective in 50% of the population and LD₅₀—the dose lethal to 50% of the population) of an antibody may be determined by standard pharmaceutical procedures in cell cultures or experimental animals. The dose ratio of toxic to therapeutic effects is the therapeutic index, and it may be expressed as the ratio, LD₅₀/ED₅₀. The data obtained from animal studies may used in formulating a range of dosage for human use. The dosage contained in such compositions may be within a range of circulating concentrations that include the ED₅₀ with little or no toxicity. The dosage varies within this range depending upon the dosage form employed, sensitivity of the patient, and the route of administration.

The exact dosage may be determined by the practitioner, in light of factors related to the patient who requires treatment. Dosage and administration may be adjusted to provide sufficient levels of the antibody or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy.

The antigens and/or antibodies of the invention may also be administered by introducing genetically engineered bacteria which express and release the expressed antigens and/or antibodies of the invention once the bacteria are present in the patient. This format might be suitable for treating HIV infections. The antigen and/or antibody-expressing bacteria can be introduced into mucus membranes of the throat, for example, or in other mucosal regions in which HIV might be found. Methods for constructing and/or engineering such recombinant bacteria are well known in the art.

Polynucleotides encoding the antigens and/or antibodies of the invention may be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antigen and/or antibody are in the range of about 5 μg to about 500 μg/kg of patient body weight. For administration of polynucleotides encoding the antibodies, effective in vivo dosages are in the range of about 100 ng to about 500 μg of DNA.

The antigens and/or antibodies of the present invention can also be delivered in a microsphere or microsome bodies.

The mode of administration of antigen- and/or antibody-containing pharmaceutical compositions of the present invention may be any suitable route which delivers the antibody to the host. As an example, pharmaceutical compositions of the invention may be useful for parenteral administration (e.g., subcutaneous, intramuscular, intravenous, or intranasal administration, or microsomal or lipid microsome bodies).

The above disclosure generally describes the present invention. A more complete understanding can be obtained by reference to the following specific examples, which are provided for purposes of illustration only and are not intended to limit the scope of the invention.

### EXAMPLES

The structures, materials, compositions, and methods described herein are intended to be representative examples of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

### Example 1

### HIV bcrnAbs Show Extensive Somatic Mutational Diversification in Contrast to bcrnAbs Against Henipaviruses and the SARS CoV which Cause Acute Infections

In spite of the tremendous amount of work on HIV-specific antibodies, there are relatively few articles that provide an analysis of their sequences. Early studies have found relatively extensive antigen-driven maturation and nonrestricted use of the V genes in several HIV-specific antibodies (Felgenhauer et al., 1990; Andris et al., 1991; Marasco et al., 1992; Moran et al., 1993). Other early studies of other infectious agents, e.g., Haemophilus influenzae, demonstrated that the human antibody response to type b polysaccharide of H. influenzae involves restricted VH gene usage (Scott et al., 1989). The VL response to H. influenzae shows two distinct populations, one that has little or no somatic mutation and a second, less frequent population of multiple VL genes with significant mutations, mainly in the CDRs (Scott et al., 1991). Later, an analysis of non-neutralizing HIV gp41-specific human antibodies showed an average mutation extent of about 10% (Binley et al., 1996). A recent study of the gene usage and extent of maturation of CD4-induced (CD4i) antibodies suggested a restricted VH1-69 gene usage for CD4i antibodies with long CDR3 and VH1-24 for antibodies with short CDR3s (Huang et al., 2004). It was noted in this study that two of the best characterized anti-gp120 bnAbs, b12 and 2G12, showed at least 44 and 51 somatic mutations (total for heavy and light chain V and J genes), respectively, which is more than 20% and double the average number of somatic mutations (22+1-6) observed for the other gp120-reactive antibodies analyzed in the study (see Table 1 in (Huang et al., 2004)); it was also noted that this high number of mutations compared to the germline may explain in part their rarity.

TABLE 1
Extent of maturation for HIV-1 specific antibodies

| | Mutation by (VH) | | | Mutation by (VL) | | | Mutation | | | |
|---|---|---|---|---|---|---|---|---|---|---|
| | | Non | | | Non | | as residues | | | % |
| Abs | Silent | silent | Total | Silent | silent | Total | VH | VL | Total | change |

gp120-specific broadly cross-reactive neutralizing antibodies

| | | |
|---|---|---|
| b12 | 45 | 18.3 |
| 2G12 | 51 | 20.7 |
| | Aver. | 19.5 |

gp41-specific cross-reactive antibodies

| | | | | | | | | | | |
|---|---|---|---|---|---|---|---|---|---|---|
| m43 | 5 | 28 | 33 | 3 | 15 | 18 | 20 | 14 | 34 | 15.7 |
| m44 | 13 | 29 | 42 | 8 | 24 | 32 | 21 | 16 | 37 | 17.1 |
| m45 | 11 | 20 | 31 | 13 | 29 | 42 | 17 | 17 | 34 | 15.7 |
| m46 | 14 | 39 | 53 | 5 | 15 | 20 | 27 | 12 | 39 | 18.0 |
| m47 | 2 | 34 | 36 | 1 | 9 | 10 | 20 | 8 | 28 | 12.9 |
| m48 | 8 | 39 | 47 | 1 | 9 | 10 | 24 | 7 | 31 | 14.3 |
| | | | | | | | | | Aver. | 15.6 |

gp120-specific CD4-induced antibodies

| | | | | | | | | | | |
|---|---|---|---|---|---|---|---|---|---|---|
| 412d | 2 | 9 | 11 | 1 | 8 | 9 | 8 | 8 | 16 | 7.4 |
| E51 | 6 | 18 | 24 | 3 | 5 | 8 | 17 | 5 | 22 | 10 |
| 47c | 2 | 7 | 9 | 1 | 1 | 2 | 6 | 1 | 7 | 3.2 |
| 48d | 8 | 12 | 20 | 5 | 5 | 10 | 12 | 5 | 17 | 7.9 |
| 16c | 5 | 9 | 14 | 3 | 4 | 7 | 8 | 4 | 12 | 5.6 |
| 411g | 3 | 7 | 10 | 1 | 9 | 10 | 6 | 9 | 15 | 6.9 |
| 23e | 7 | 13 | 20 | 0 | 4 | 4 | 10 | 3 | 13 | 5.9 |
| 4KG5 | 7 | 24 | 31 | 4 | 9 | 13 | 17 | 6 | 23 | 10.6 |
| X5 | 5 | 14 | 19 | 4 | 7 | 11 | 13 | 6 | 19 | 8.8 |
| m16 | 12 | 33 | 45 | 2 | 3 | 5 | 25 | 3 | 28 | 13.1 |
| 17b | | | | | | | | | 27 | 11 |
| | | | | | | | | | Aver. | 8.2 |

The present inventor has identified several hmAbs which exhibit cross-reactive neutralizing activity clades (Zhang et al., 2003; Moulard et al., 2003; Zhang et al., 2004a; Zhang et al., 2004b; Zhang et al., 2004c; Choudhry et al., 2006; Zhang et al., 2006; Choudhry et al., 2007; Zhang & Dimitrov, 2007) (Zhang, Alam, Haynes, Dimitrov et al., AIDS Vaccine 2007, Late Breaker Presentation). The extent of maturation of the gp41-specific hmAbs m43-m48 were analyzed and compared with that of the two best characterized potent bcrnAbs, 2F5 and 4E10. As shown in the below table, these newly identified antibodies contain numerous mutations compared to the closest germline sequences; the extent of maturation on average is comparable to that of 4E10 and higher than that of 2F5 (note that the number of mutations in the table are for the VH and VL genes, not for the V plus J genes). The extent of maturation for some of the antibodies, e.g. m46 and m44, is comparable to that of b12.

TABLE 2
Extensive somatic mutational diversification of HIV-1-neutralizing
cross-reactive antibodies.

| Abs | VL | VH |
|---|---|---|
| m43 | 14 | 20 |
| m44 | 12 | 21 |
| m45 | 16 | 17 |
| m46 | 12 | 27 |
| m47 | 8 | 22 |
| m48 | 7 | 24 |
| 2F5 | 14 | 16 |
| 4E10 | 13 | 20 |
| b12 | 20 | 23 |
| 2G12 | 13 | 32 |

Similarly, the 120-specific CD4bs antibodies, m14 (Zhang et al., 2004c) and m18 (Zhang et al., 2003), were identified as also being extensively matured. Antibody m18 is comparable to b12 in that aspect, which could be related to its unique structure mimicking the receptor CD4 (Prabakaran et al., 2006b). Note also that for all antibodies in these two groups the heavy chain is significantly more diversified than the light chain.

Importantly, the CD4i antibodies are significantly less matured than the bnAbs targeting gp41 and the CD4 binding site on gp120. On average, their extent of maturation as estimated by the total number of amino acid mutations in the VH and VL genes is about 2-fold lower than for the bcrnAbs. These antibodies in IgG format are weak neutralizers with limited breadth of neutralization likely due to steric restrictions of the access to the coreceptor binding site (Labrijn et al., 2003). Note, however, that although binding of m16 to gp120 is increased when complexed with CD4 (Zhang et al., 2004b), it binds also gp120 alone and neutralizes isolates from different clades even in its IgG1 format (Dimitrov et al., unpublished data); thus, m16 is not a typical CD4i antibody as, e.g., the prototype 17b.

The CD4i antibodies target a highly conserved and immunogenic structure overlapping with the coreceptor binding site; also they are abundant (Decker et al., 2005). A question arises as to why they are not so extensively matured as the bnAbs. One possibility is that indeed their level of maturation is at an average level typical for immune responses to other antigens. HIV has not evolved to develop protective mechanisms for the coreceptor binding site because it is not accessible to IgGs and the antibodies against it are not neutralizing or only weakly neutralizing although exceptions are possible; thus they don't require extreme levels of maturation to overcome the HIV immune response evasion mechanisms. One must emphasize that the 2-fold difference in extent of maturation as measured by number of amino acid residue changes from the closest germline sequence is related in a complex, stochastic way to the time required to reach levels of maturation that differ by 2-fold; it could take much longer than a simply 2-fold increase in time to elicit specific antibody that has 2-fold more mutations than another one.

Because of the exponential combinatorial increase of the space of possible mutations in the pathways to maturation, a 2-fold increase in the number of mutations could require much longer time than a simple 2-fold increase. It could take 10 or 100-fold longer times; it is impossible to calculate theoretically but simple considerations indicate a possibility for very long times on the order of years to reach 30-50 mutations. If this is true, it would suggest that it could take years to elicit bcrnAbs with activity similar to b12, e.g., by using current practices of immunization. Thus it is important to perform systematic study of the extent of maturation and gene usage for large number of HIV-specific antibodies and derive conclusions with high power of statistical significance.

There are two sets of recent data that also seem to support the possible importance of the extent of maturation for HIV neutralization: an analysis of sequences of antibodies selected by screening of nonimmune phage libraries and antibody sequence analysis for viruses causing acute infections. Significantly lower level of maturation compared to that for bnAbs was observed for antibodies of limited cross-reactivity and potency selected from a large (10¹⁰ antibodies) nonimmune library developed in Dimitrov group (Table 3). The average total percentage change for both heavy and light chain was 7% in the range from 0.5 to 12. Note, however, that the heavy chain which is typically determining the antigen specificity is significantly less matured than the light chain—on average 4% in the range from 0 to 8%. It is likely that some of the individuals the library was made were previously infected and had antibodies with extensively matured light chains that could contribute to an increased affinity; the process of library panning indeed leads to selection of such antibodies of highest affinity. An additional factor is that to mimic better an initial response during immunization by in vitro screening of nonimmune libraries, the heavy chains of the antibodies in the library are of M type corresponding to IgMs which typically don't undergo such extensive SHM as IgGs although they do undergo SHM but likely not in the GC (Weller et al., 2004; Weller et al., 2005).

TABLE 3
Relatively low level of maturation of HIV-1 specific antibodies of limited
cross-reactivity and potency selected from a large non-immune library

| Antibodies | VL | VH | Total | % change |
|---|---|---|---|---|
| m1n | 1 | 6 | 7 | 4 |
| m2n | 1 | 0 | 1 | 0.5 |
| m3n | 0 | 4 | 4 | 2 |
| m4n | 15 | 4 | 19 | 10 |
| m5n | 24 | 3 | 27 | 14 |
| m6n | 13 | 2 | 15 | 8 |
| m7n | 16 | 8 | 24 | 12 |

In contrast to HIV, the use of the same nonimmune library for panning with an envelope glycoprotein fragment (receptor-binding domain) from the SARS CoV resulted in a very potent bcrAb, m396, which neutralized all isolates tested including some from animals, and based on the crystal structure and known sequences is likely to neutralize all isolates (about 100) with known sequences. Note that because the SARS CoV is an RNA virus it also extensively mutates in spite of the relatively short duration of the infection and the epidemic. Panning of the same library also resulted in potent cross-reactive antibodies against Nipah and Hendra viruses. The extent of maturation of these antibodies was low (on average 5%) (Table 4). Note that the anti-SARS CoV antibody m396 has low level of maturation (3%) and its light chain is virtually identical to the germline. Several other SARS CoV-specific antibodies (Sui et al., 2004) identified in Marasco group have somewhat higher level of maturation (about 10%) but still significantly lower than that of the bnAbs against HIV (data not shown). Note that these antibodies are not cross-reactive but have about the same level of maturation although the light chains are more extensively matured. Thus it appears that for viruses which have not elaborated extensive mechanisms of protection from immune responses the level of maturation is not necessary to be very high to achieve high and broad neutralizing activity, and in general the extent of maturation doesn't correlate with neutralizing activity. An analysis of antibody sequences in humans immunized with tethanus toxoid (Meijer et al., 2006) also revealed similar extent of maturation, in general about 10%, i.e., about 2-fold lower than that for bnAbs against HIV.

TABLE 4
Relatively low level of maturation of potent cross-reactive antibodies
against SARS CoV, Nipah and Hendra viruses selected
from a large non-immune library

| Antibodies | VL | VH | Total | % change |
|---|---|---|---|---|
| m101 | 9 | 1 | 10 | 5 |
| m102 | 8 | 6 | 14 | 7 |
| m106 | 0 | 1 | 1 | 0.5 |
| m396 | 2 | 5 | 7 | 3 |

Antibodies against other pathogenic microbes selected from the same library also have relatively low level of maturation (on average 3%) (Table 5).

TABLE 5
Relatively low level of maturation of antibodies specific for other
pathogenic microbes selected from a large non-immune library

| Antibodies | % change |
|---|---|
| Henipaviruses | 4 |
| CCHFV | 2.5 |
| Vaccinia | 4.5 |
| Monkeypox virus | 1 |
| Yersinia pestis | 4 |

These results indicate that it is possible to select potent cross-reactive antibodies with low level of maturation against some pathogens but not against HIV-1. In a preliminary attempt to reconstitute the pathways of maturation Dimitrov and his associates also amplified from an immune library a number of clones with sequences similar to those of the cross-reactive HIV-1-neutralizing antibody, m14, by using primers specific for the CDR3 of its heavy chain (H3). In another study (Choudhry et al., 2007) the inventor and his associates found tens of antibodies with H3s similar or identical to those of m14 and another bcrAb, m18, but with a number of mutations in the other variable regions and in the frameworks; some of the mutations were repeated (hot spots). The analysis of the pathways of maturation could provide important clues of how bnAbs against HIV are developed in vivo.

### Example 2

### Antibody Diversity

An estimate of the germline antibody diversity in humans based on the number of different antibodies that could be formed from the germline V, D, and J sequences is known to be about 10⁴ combinations for the heavy chain and several hundred for the light chain (Max, 2003). This estimate assumes that there are 40 VH regions, 27 D regions, and 6 JH regions, resulting in 6,480 possible combinations for the heavy chain. If the three reading frames available for the D regions are taken into account, the total comes to 19,440 combinations of amino acid sequences. However, at least in one of the reading frames there are numerous stop codons. Thus the actual number for the heavy chain could be on the order of 10⁴. For the light chain, there are 145 κ combinations (29 Vκ×5 Jκ) plus 120% combinations (30 Vλ×4 Jλ), or 265 total light-chain combinations.

Other estimates have yielded similar although not identical estimates, e.g. 1.1×10 4 variable domain heavy chains and 320 light chains—see e.g. a citation in a recent article (Clark et al., 2006). If the pairing of the heavy and light chains occurs randomly, several million combinations could be calculated. This estimate has neglected additional sources of diversity that are difficult to estimate, including e.g. the insertion of N and P nucleotides. However, this vast combinatorial diversity could not be entirely functional in vivo. It seems unlikely, for example, that every possible combination of light and heavy chains yields a functional antibody molecule, because in vitro light- and heavy-chain reassociation experiments show that certain hybrid molecules (formed from light and heavy chains derived from different antibodies) are relatively unstable. Similarly, association of V and J (or V, D, and J) is conceivably not completely random. In addition, fetal and newborn V-D-J junctions show a paucity of N nucleotides and a tendency to form V-D-J junctions across short stretches of sequence identity between the recombining sequences (“homology mediated” recombination).

Experimental verification of these type of estimates which would require sequencing of thousands of antibodies has never been reported in spite of its fundamental significance. Of note is that the actual number of antibody genes in the human genome is significantly higher, but only a portion of them are functional. There are no experimental studies of what is the total number of productive antibodies with recombined heavy chains and light chains.

Thus, this example outlines a strategy to measure the number of expressed heavy and light chain variable domains.

Two libraries were prepared from cord blood which appear to contain mostly germline sequences. These libraries could be a starting point for evaluation of the expressed germline repertoire in humans. DNA minipreps from these libraries will be prepared and sequenced.

Somatic hypermutation can generate a number of mutants limited only by the number of B cells which for adult humans is on the order of 10¹⁰. Thus the germline repertoire is limited and additional diversification could not lead to more sequences than the number of B cells. This suggest that “holes” in the germline repertoire as well as in the acquired repertoires are possible and they could be used by HIV to evade immune responses against its vulnerable functionally important epitopes that bind bcrnAbs.

### Example 3

### Identification of the Maturational Pathway or Portions of Maturational Pathways for One or More Specific Anti-HIV bcrnAb

A direct approach to identify portions of plausible pathways of maturation of antibodies with known sequences is to obtain B cell samples from non-infected and non-immunized humans that could be analyzed for antibodies that are close in sequence to those germline sequences that correspond to the sequences of the known bcrnAbs. Such antibodies are expressed, purified and characterized in terms of their binding ability which typically should be very low even to oligomeric Env. Even better approach although more difficult is to obtain sequential samples from HIV-infected individuals with high levels of bcrnAbs and analyzed them as above; sequential samples from any infected or immunized human could be also useful although the probability to obtain information for maturational pathways of bcrnAbs would be lower. Two other approaches are based on antibody libraries as a source. One approach to identify possible maturational pathways is to explore the source library for the bcrnAb of interest, and by using PCR with primers against the CDR3 of the heavy chain to identify a panel of antibodies with almost identical CDR3s; similarly for the light chain with primers against the CDR3; other CDRs could also be used. The resulting sequences are analyzed and plausible pathways are reconstituted. Note that multiple pathways are possible; increasing the number of possible pathways increases the immunogenicity of the antigen that leads to maturation of the antibody. Second approach is based on the generation of a library of all possible mutants from the corresponding germline. The library is screened against the HIV Env and the resulting antibodies are arranged according to their increasing affinity. Those antibodies with intermediate number of mutations that show some binding to the Env will be selected and used as intermediate antibodies. Good intermediate antibodies are those which are as close to the germline as possible and still show some (typically small) affinity (avidity) for the epitope of the bcrnAb to be elicited.

### Example 4

### The Identification of a Panel of Antibodies Having an Intermediate Extent of Somatic Mutational Diversity

Intermediate antibodies are those antibodies in the maturational pathway of a matured antibody identified as described above that have one or more mutations but less mutations than the corresponding desired mature antibody, e.g., a bcrnAb. Typically such antibodies have several mutations from the closest corresponding germline antibody. They will be identified by their relatively weak binding to the Env even in a bivalent (e.g. IgGs) or multivalent formats. They will be further characterized and produced in quantities sufficient for the identification of corresponding primary antigens/immunogens.

### Example 5

### Screening and Identification of Primary Antigen/Immunogen

The primary immunogen(s) can be any immunogen that binds the germline antibody corresponding to the antibody, e.g., a bcrnAb, of interest and binds also the intermediate antibody (see slide 2 from the power point file). It can be identified by using the intermediate antibody as a tool. Libraries of proteins, antibodies and peptides can be screen against the intermediate antibody(ies) for identification of candidate antigens that bind with high affinity to the intermediate antibody and with lower affinity to the corresponding germline antibody. For example, one can screen phage-displayed libraries of human CH2 domains with randomly mutated loops or with grafted CDRs from antibody libraries against the intermediate antibody. This system has advantage that the framework is human and may not induce immune response if used as vaccine immunogen. In addition, it could have prolonged half-life in the circulation. Another example is panning of libraries of phage-displayed peptides against the intermediate antibody. The selected peptides could be used as primary immunogens. A variation of this method is to use libraries of peptides based on randomly mutated MPER (membrane proximal external region)—two of the known bcrnAbs (2F5 and 4E10) bind to MPER thus the intermediate antibody could cross-react and bind weakly to portions of their epitopes. cDNA libraries of human and non-human proteins can be also screened with the intermediate antibody. The selected primary immunogen must also bind the germline antibody although typically with low affinity even in multivalent format in order to be able to initiate immune response. It should be constructed to have minimal number of epitopes different from those binding to the germline and the intermediate antibody, i.e. to focus the immune response on the important epitopes.

### Example 6

### Identification of the Epitopes on the Primary Antigen/Immunogen

The epitopes of the germline and intermediate antibodies on the primary immunogen can be identified by using standard methods including competitive binding of known antibodies with known epitopes in scFv or Fab formats to avoid steric hindrance as much as possible. Whenever possible, the best method is to co-crystallize the germline and intermediate antibodies (typically antibody fragments, Fab or scFvs) with the primary immunogen. This would allow to develop structures that are devoided of most of the other possible epitopes and focus the immune response on elicitation of the intermediate antibody. Regions of these structures could mimic portions of the epitope of the final matured antibodies because the intermediate antibody is cross-reactive with the secondary immunogen (e.g. the Env).

### Example 7

### Construction of Primary Immunogen Sufficient to Elicit the Intermediate Antibody

The construction of the primary immunogen is based on the identification of a panel of possible primary immunogens as described above. Knowledge of the epitopes of the germline and intermediate antibody could help to design primary immunogen containing structures with exposed epitopes for these antibodies and lacking as much as possible other epitopes. Such immunogens could be produced as DNA vaccines or proteins or protein fragments using standard methods.

### Example 8

### Administration of Combination of Primary Immunogen and HIV Immunogen to Elicit bcrnAb in Test Animal

The primary and secondary immunogens can be administered simultaneously or sequentially. Because the first somatic mutational diversification events can occur typically in a week, therefore, if the secondary immunogen is administered sequentially it should be administered in a week or two, and typically not later than a month. In case that the primary immunogen is constructed as a long-lived molecule in the circulation, e.g., by making a fusion protein with Fc, then sequential administration could have some advantages. However, probably the simplest and most effective way is if the primary and secondary immunogens are fused in one molecule and that molecule is fused e.g. with Fc to increase the half-life in vivo and to allow binding to receptor on immune cells. The immunogens are administered using standard protocols and adjuvants.

### Example 9

### Germline-Like Predecessors of Broadly Neutralizing Antibodies Lack Measurable Binding to HIV-1 Envelope Glycoproteins

Potent broadly cross-reactive neutralizing antibodies (bnAbs) 48 are relatively rarely found in patients with HIV-1 infection. Possible 49 causes include protection of conserved structures of the virus envelope glycoprotein (Env) by variable loops, extensive glycosylation, occlusion within the oligomer, and conformational masking, as well as the rapid generation of HIV-1 mutants that outpace the development of such antibodies and immunoregulatory mechanisms.

The Env is immunogenic and a number of Env-specific hmAbs have 54 been identified. However, only several hmAbs, including IgG b12, IgG 2G12, and IgG 2F5, have been extensively characterized and found to exhibit relatively potent and broad neutralizing activity to isolates from different clades. The existence of these antibodies has fueled the hope that the development of efficacious HIV vaccine is achievable provided that an immunogen containing the epitopes of these antibodies is appropriately designed.

However, in spite of the large amount of research an antibody-based vaccine capable of eliciting broadly neutralizing antibodies has not been achieved. The inability to achieve elicitation of such bnAbs in humans indicates that there are still unknown fundamental immunological mechanisms that allow HIV to evade elicitation of bnAbs.

Previous studies have found relatively extensive antigen-driven maturation and non-restricted use of the V genes in several HIV-specific antibodies. Later, an analysis of non-neutralizing HIV gp41-specific human antibodies showed an average mutation frequency of approximately 10% (Binley et al., 1996). A more recent study of the gene usage and extent of maturation of CD4-induced (CD4i) antibodies suggested a restricted VH1-69 gene usage for CD4i antibodies with long CDR3 and VH1-24 for CD4i antibodies with short CDR3s (Huang et al., 2004). It has been observed that two of the best characterized anti-gp120 bnAbs, b12 and 2G12, have nearly 2-fold higher somatic hypermutation (about 20% mutation frequency) than other gp120-reactive antibodies.

This Example investigates whether the high divergence of the known bnAbs from their corresponding germline antibodies may indicate that the germline antibodies lack the capability to bind the epitopes of the mature antibodies. Germline-like antibodies corresponding to b12, 2G12, and 2F5, as well as antibodies to several human HIV-1-specific hmAbs (X5, m44, and m46) were designed for this Example.

Fab X5 is a potent CD4i bnAb but as a full-size (IgG1) antibody exhibits on average significantly decreased potency likely due to size-restricted access to its epitope. IgG1 m44 and IgG1 m46 are gp41-specific cross-reactive HIV-1-neutralizing hmAbs with relatively modest potency. Germline-like b12, 2G12 and 2F5 did not bind to any of the Envs although the corresponding mature antibodies did bind with relatively high level of activity. In contrast the germline-like X5, m44, and m46 bound with relatively high affinity to all tested Envs. These results provide initial evidence that germline-like antibodies corresponding to known bnAbs antibodies may not be capable of binding to the Env to initiate and/or maintain an immune response leading to their elicitation in vivo.

### Materials and Methods

Analysis of Antibody Sequences and Design of Germline-Like Anti-Bodies.

The heavy and light chain nucleotide sequences were analyzed with JOINSOLVER®. The mAb V(D)J alignments were assigned to the germline gene that yielded the fewest nucleotide mismatches. Values of p<0.05 were used to compare D segment alignments to that expected from random chance. The minimum requirement for D segment alignment was 9 or 10 (depending on the length of the V to J region) matching nucleotides and at least 2 additional matches for every mismatch. Germline-like sequences were determined by reverting mutations to the germline sequence while retaining the original CDR3 junctions and terminal deoxynucleotidyl transferase (TdT) N nucleotides.

Gene Synthesis and Expression Plasmid Constructions.

ScFv DNAs corresponding to mature and germline-like X5, m44, m46, b12, 2G12, and 2F5 were synthesized by Genescript (Genescript, Piscatawy, N.J.) and their accuracies were confirmed by sequencing. The VH of each of the antibodies was followed by a (GGGGS)₃ linker and the VL. SfiI restriction site was added to both N and C termini for each scFv during gene synthesis for cloning into pCOM3X plasmid for expression in bacteria. The pCOM3X vector adds a His tag to the C terminus of each inserted scFv. The His tag was used subsequently for scFv purification and detection in ELISA. The DNA fragments encoding selected scFv antibodies were fused with Fc of human IgG1 and cloned into the mammalian cell expression vector pSecTag2B (Invitrogen, Carlsbad, Calif.) for expression of the fusion proteins.

Antibody Expression and Purification.

For scFv expression, Escherichia coli strain HB2151 was transformed by the scFv constructs described above. A single clone was inoculated into 2YT supplemented with 100 U of ampicillin, 0.2% glucose and incubated at 37° C. with shaking When the OD600 reached 0.9, IPTG was added to achieve a final concentration of 1 mM and the culture continued overnight at 30° C. with shaking Cells were then collected, lysed with polymyxin B (Sigma, St. Louis) in PBS, and the supernatant was subjected to the Ni-NTA agarose bead (Qiagen, Hilden, Germany) purification for the soluble scFvs. The scFv-Fc constructs were transfected into the 293 freestyle cells with polyfectin transfection agent (Invitrogen). Four days after transfection, the culture medium was collected and the secreted scFv-Fc proteins were purified using a protein-A Sepharose column (GE Healthcare, Piscataway, N.J.).

ELISA.

Protein antigens diluted in PBS buffer in concentrations ranging from 1 to 4 μg/ml were added to the 96 well plate and left at 4° C. overnight to coat the plate. The plate was then blocked with PBS+5% dry milk buffer. ScFv and scFv-Fc in different concentrations were diluted in the same blocking buffer and applied to the ELISA plate. The mouse-anti-His-HRP was used to detect the His tag at the C terminus end of each of the scFv clones and the mouse-anti-human Fc-HRP was used to detect the Fc tag of the scFv-Fcs in most of the ELISA unless indicated otherwise. The HRP substrate ABTS (Roche, Mannheim, Germany) was then added to each well and OD 405 was taken 5-10 min afterward.

### Results

High Divergence of HIV-1-Neutralizing hmAbs from Germline Antibodies.

This Example has identified and characterized a number of hmAbs against HIV-1, some of which exhibit cross-reactive neutralizing activity against primary isolates from different clades as well as a number of hmAbs against the SARS CoV, Hendra and Nipah viruses. One of the antibodies (m396) potently neutralizes SARS CoV isolates from humans and animals and others (m102 and m102.4) both henipaviruses, Nipah and Hendra. The identification of many hmAbs against various infectious agents has provided an opportunity to analyze and compare their antibody sequences.

The closest germline Ig genes were identified and the antibody gene divergence was calculated as the number of amino acid changes from the corresponding germline antibodies (using mostly the VH gene for comparison). We found that all of our HIV-1 specific antibodies and three bnAbs with publicly available DNA sequences, b12, 2G12 and 2F5, were hypermutated more than normal donor memory B cells which average 13 mutations per VH sequence (FIG. 7 and data not shown). In contrast, the antibodies against the SARS CoV and henipaviruses including m396, m102, and m102.4 had only several mutations from the closest germline (on average <5%, data not shown). Potent antibody against a bacterial pathogen (Yersinia pestis) also had relatively low (3%) number of mutations (data not shown).

These results indicate that bnAbs against HIV-1 are significantly more divergent from the closest germline antibodies than hmAbs against SARS CoV and henipaviruses with potent and broad neutralizing activity.

Design of Germline-Like X5, m44, m46, b12, 2G12, and 2F5.

To test whether the closest germline-like antibodies that presumably initiated the hypermutation process can bind the Env, corresponding germline-like antibodies were designed (FIG. 7). Because of the diversity of the D segment in the heavy chain CDR3 (H3) of m44, m46, b12, and 2G12 the germline sequence could not be determined with 95% confidence and the original D segment amino acid sequence was used for synthesizing the germline-like Ab.

Germline-Like scFvs X5, m44, and m46 Bind but b12, 2G12, and 2F5 Lack Measurable Binding to Envs

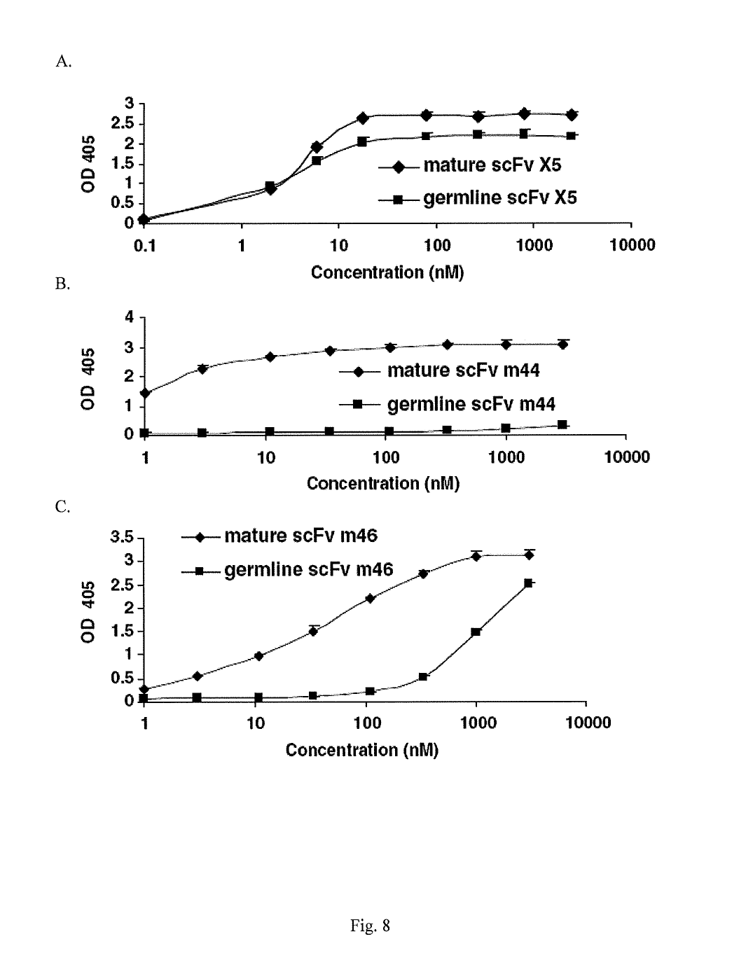

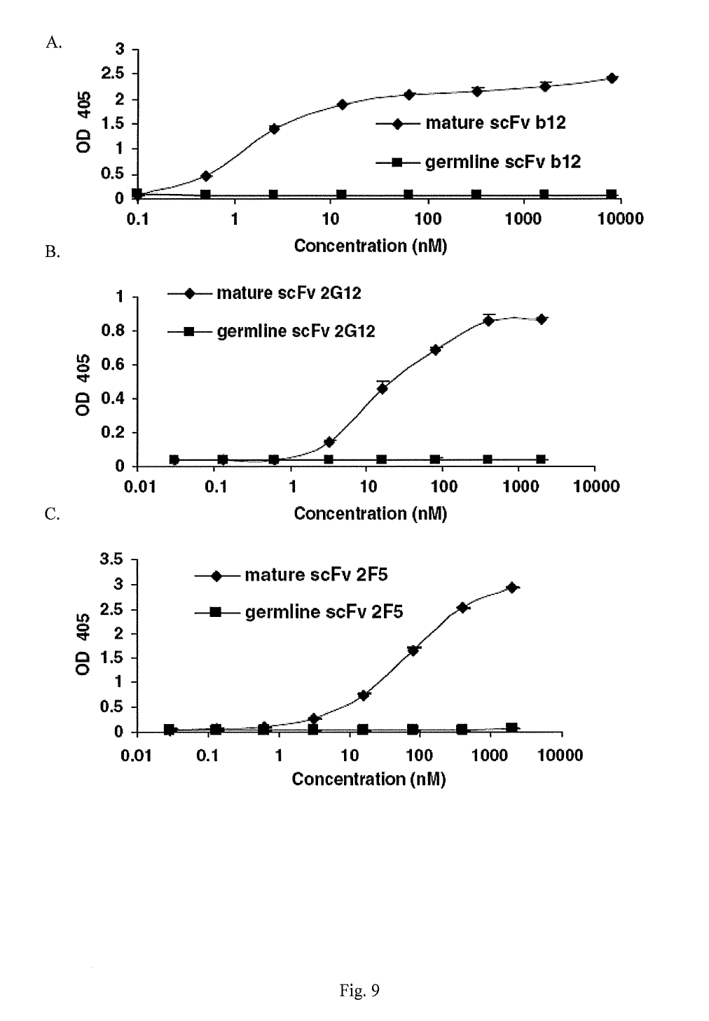

To explore the hypothesis that some germline antibodies against conserved epitopes may not bind structures containing epitopes of their corresponding mature antibodies, genes for six germline-like antibodies were synthesized in a scFv format. The purified scFvs were tested for binding in an ELISA assay where recombinant Envs (gp140s) were used as target antigens. High affinity binding of germline-like X5 and lower affinity binding for the germline-like antibodies m44 and m46 were observed (FIG. 8). In contrast, there was no measurable binding for the germline-like antibodies b12, 2G12, and 2F5 even at very high (1M range) concentrations (ELISA signal at or below negative control with irrelevant antigens) (FIG. 9).

These results demonstrate that the germline-like antibodies corresponding to these three antibodies do not bind to recombinant gp140 in our ELISA assay even at high concentrations.

Bivalent Fc Fusion Proteins of Germline-Like b12, 2G12, and 2F5 Lack Measurable Binding to Envs

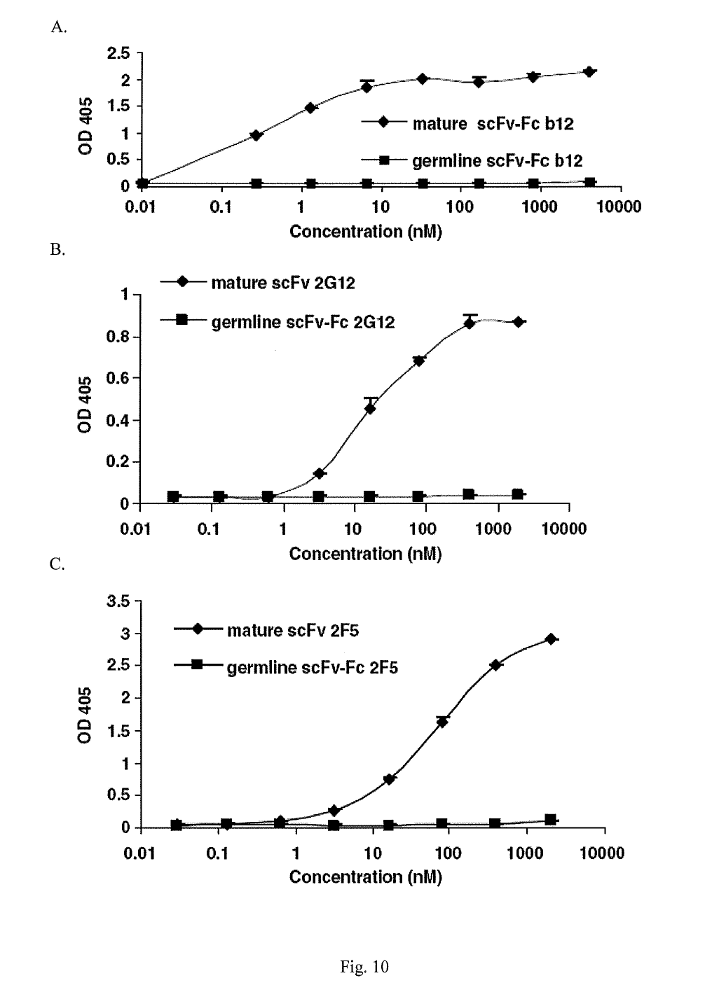

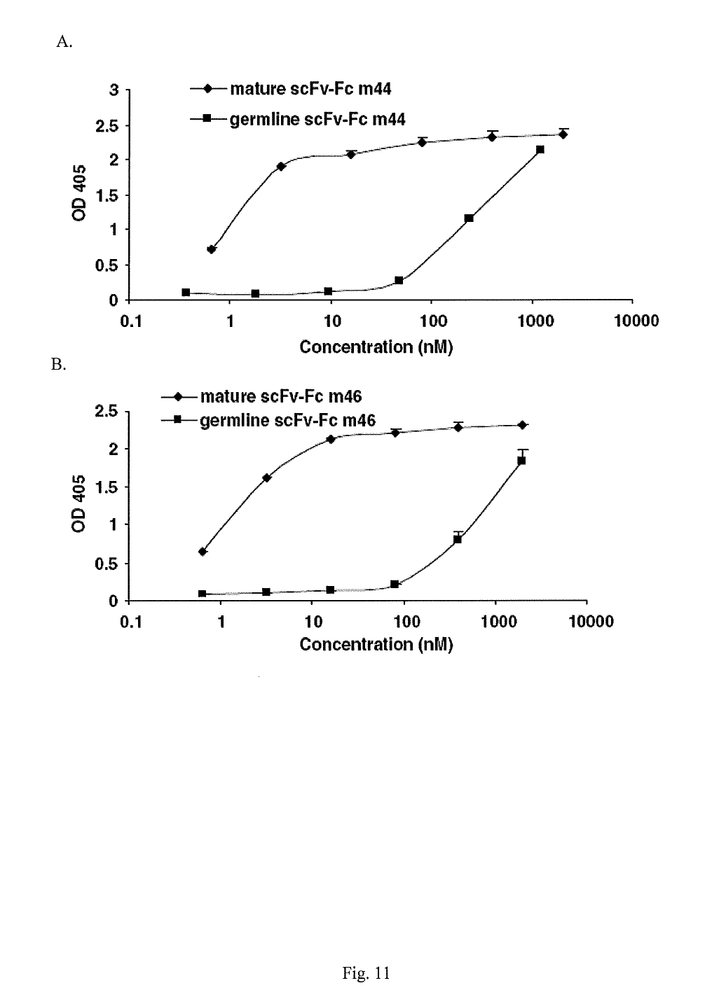

To test whether avidity effects could lead to measurable binding of the germline-like b12, 2G12, and 2F5, bivalent scFv-Fc fusion proteins were constructed, expressed and purified. These antibodies did not exhibit measurable binding in the same ELISA assay even at very high (1M range) concentrations (FIG. 10). As expected, due to avidity effects the binding of the Fc fusion proteins with germline-like m44 and m46 was enhanced (FIG. 11). These results indicate that bivalent avidity effects do not lead to measurable binding of germline-like b12, 2G12, and 2F5 in our ELISA assay.

### Discussion

A number of HIV-1-specific neutralizing antibodies have been found to have unusually high frequencies of somatic hypermutation. The increase in somatic hypermutation was associated with an increase in nonsynonymous amino acid substitutions. In contrast, the neutralizing hmAbs against several viruses causing acute infections contain fewer amino acid substitutions. Notably, the potent bnAbs against SARS CoV and henipaviruses were selected by screening a large non-immune antibody library derived from ten healthy volunteers against the respective Envs, as a method method for resembling to a certain extent in vivo immunization. To better mimic the B cells that respond to primary immunization, the heavy chains of the antibodies in this library from normal donors were of 1 type corresponding to IgM+B cells. When the same library and screening methodology was used against HIV-1 Envs, only weakly neutralizing non-cross-reactive antibodies resulted (data not shown). Panning with another IgM library from large number of healthy individuals resulted in non-neutralizing or even infection-enhancing antibodies (data not shown).

Previous attempts to select HIV-specific antibodies from non-immune libraries have also resulted in antibodies with modest neutralizing activity and limited breadth of neutralization. This Example indicates that HIV-1 has developed a strategy to protect its highly conserved epitopes against initial immune responses. In contrast, SARS CoV and henipaviruses appear to lack such a mechanism and their Envs contain exposed, conserved receptor binding sites that can bind IgM+B cells with sufficient affinity to induce class switch and affinity maturation. Therefore, unlike HIV-1, Env-based vaccine immunogens and in particular the receptor binding domains of SARS CoV and henipaviruses can be highly effective in eliciting bnAbs.

Further support for this line of reasoning is that germline-like b12, 2G12, and 2F5 lack measurable binding to Envs, as shown here. No binding was detected even at relatively high (up to 10 μM) antibody concentrations. Although in general the threshold for B cell activation is believed to be on the order of μM equilibrium dissociation constants, it was demonstrated that even lower affinity/avidity interactions can trigger B cell activation in mice. However, even if binding occurs with very low avidity activated B cells expressing such BCRs are likely to be outcompeted by B cells expressing BCRs that bind to other epitopes with higher affinity/avidity. Such epitopes include those of X5 as a representative of a CD4i epitope and m44 and m46 as representatives of gp41 epitopes. X5 and other CD4i antibodies target a highly conserved and immunogenic structure overlapping with the coreceptor binding site; such antibodies are abundant in patients with HIV-1 infection. It has been demonstrated that the differences in responses of high and low affinity B cells can be relatively small but in competition experiments only the high-affinity B cells respond to antigen.

It can be hypothesized that during lengthy chronic infections, HIV has evolved mechanisms to protect its most vulnerable but functionally important conserved structures including the CD4 binding site, conserved carbohydrates and gp41 membrane proximal external region (MPER) by using “holes” in the human germline BCR repertoire, i.e., these structure do not bind or bind very weakly to germline antibodies. At the same time HIV has evolved other structures which are either not accessible for full-size antibodies (e.g., some CD4i epitopes including the X5 one) or are not functionally important but can bind with relatively high affinity to B cells expressing germline antibodies that can out-compete those B cells expressing BCRs against conserved epitopes, if any.

In conclusions, the results here indicate another possible mechanism used by HIV-1 to evade neutralizing immune responses. HIV-1 may be able to protect its vulnerable exposed conserved epitopes by using “holes” in the human germline repertoire. Germline BCRs that can recognize these epitopes and initiate and/or maintain immune responses by competing with BCRs that bind to other nonessential or non-accessible epitopes with high affinity may be missing from the naïve repertoire. With knowledge of this mechanim, the design of effective vaccine immunogens capable of eliciting potent bnAbs against HIV-1 may be possible.

In summary, several human monoclonal antibodies (hmAbs) including b12, 2G12, and 2F5 exhibit relatively potent and broad HIV-1-neutralizing activity. However, their elicitation in vivo by vaccine immunogens based on the HIV-1 envelope glycoprotein (Env) has not been successful. One concept is that HIV-1 has evolved a strategy to reduce or eliminate the immunogenicity of the highly conserved epitopes of such antibodies by using “holes” (absence or very weak binding to these epitopes of germline antibodies that is not sufficient to initiate and/or maintain an efficient immune response) in the human germline B cell receptor (BCR) repertoire. To test this concept germline-like antibodies were designed which correspond most closely to b12, 2G12, and 2F5 as well as to X5, m44, and m46 which are cross-reactive but with relatively weak neutralizing activity as natively occurring antibodies due to size and/or other effects. The germline-like X5, m44, and m46 bound with relatively high affinity to all tested Envs. In contrast, germline-like b12, 2G12, and 2F5 lacked measurable binding to Envs in an ELISA assay although the corresponding mature antibodies did. These results provide initial evidence that Env structures containing conserved vulnerable epitopes may not initiate humoral responses by binding to germ-line antibodies. Even if such responses are initiated by very weak binding undetectable in this assay it is likely that they will be outcompeted by responses to structures containing the epitopes of X5, m44, m46, 39 and other antibodies that bind germline BCRs with much higher affinity/avidity.

### Example 10

### Maturation Pathways of Cross-Reactive HIV-1 Neutralizing Antibodies

Elicitation of broadly cross-reactive HIV-1 neutralizing antibodies (bnAbs) in vivo is rare. This is likely due to protection of conserved structures of the virus envelope glycoprotein (Env) by variable loops, extensive glycosylation, occlusion within the oligomer, and conformational masking, and the rapid generation of HIV-1 mutants that outpace the development of such antibodies. A number of Env-specific hmAbs have been identified but only several exhibit neutralizing activity to primary isolates from different clades including IgG b12, IgG 2G12, m14, m18. 447-52D, IgG 2F5, IgG 4E10, IgG m46, IgG m48, Fab X5 and Fab Z13.

Of those, b12, 2G12, 2F5, 4E10 are best characterized and exhibit on average the broadest and most potent neutralizing activity. X5 exhibits comparable or even more potent and broad neutralizing activity which however is dependent on size—the smallest fragment (scFv) is the most potent followed by Fab and IgG. The full-size X5 antibody in the IgG1 format is significantly less potent although it can still neutralize some isolates. The existence of bnAbs suggests the possibility of the development of an efficacious HIV vaccine, provided that an immunogen containing the epitopes of these antibodies is appropriately designed.

As mentioned previously, however, the goal of an antibody-based effective vaccine based on appropriately designed and exposed or empirically found vaccine immunogen has not been achieved. The inability to achieve elicitation of such bnAbs in humans and the very low frequency of HIV-infected humans with potent bnAbs strongly suggest that there are still unknown fundamental immunological mechanisms that allow HIV to evade elicitation of bnAbs.

Example 9 analyzed the sequences of known bnAbs and found that they are highly divergent from germline antibodies. B12 is especially highly somatically hypermutated while X5 is relatively less divergent from germline antibodies. The relatively high degree of specific somatic hypermutations may preclude binding of the HIV-1 envelope glycoprotein (Env) to closest germline antibodies, and that identifying antibodies that are intermediates in the pathways to maturation could help design novel vaccine immunogens to guide the immune system for their enhanced elicitation. In support of this hypothesis, Example 9 showed a germline-like b12 (monovalent and bivalent scFv as an Fc fusion protein or IgG) to lack measurable binding to an Env as measured by ELISA with a sensitivity in the μM range. In contrast, a germline-like scFv X5 bound Env with high (nM) affinity.

This Example presents identifies possible b12 intermediate antibodies that could serve as reagents for the identification of new vaccine immunogens that can help guide the immune system through the b12 maturation pathway.

### Materials and Methods

Primers, Peptide and Proteins

Codon-optimized SCD4 D12 was cloned into the expression vector pSecTag2B (Invitrogen) attaching a His tag to the C terminus of the sCD4 D1-2, transfected into 293 freestyle cells and expressed according to the manufacturer's suggested protocol. The secreted sCD4 D12 was purified using a Nickle column from the culture medium (Qiagen, Hilden, Germany). All the primers were commercially obtained.

Gene Synthesis and Expression Plasmid Constructions

ScFv DNAs corresponding to mature and germline X5 and b12 were synthesized by Genescript (Genescript, Piscatawy, N.J.). The VH of each of the antibodies was linked to the VH via (GGGGS)₃ linker. The scFv fragment was cloned into pCOM3X for expression in bacteria. The DNA fragments encoding various b12 scFv antibodies were fused with Fc of human IgG1 and cloned into the mammalian cell expression vector pSecTag2B (Invitrogen) for expression of the scFv-Fc fusion proteins. The Vh and Vl of the germline b12 were further grafted to pDR12 vector for conversion to IgG format.

Identification of Intermediate Affinity B12 Binders

The degenerate primer B12H2 primer 5′ ATG GGA TGG ATC AAC SCT KRC AAT GGT AAC AMA AAA TAT TCA CAG 3′ was used in an overlapping PCR to replace the residues at positions 52, 53, and 57 of the germline b12 H2 with corresponding residues from the b12 mature form. A collection of germline b12 variants containing one, two or three residues from mature b12 form was generated through this process.

Antibody Expression and Purification

For scFv expression, E. coli strain HB2151 was transformed by the X5 and b12 scFv constructs described above. Single clone was inoculated into 2YT supplemented with 100 units of ampicillin, 0.2% glucose and incubated at 37° C. with shaking. When the OD600 reached 0.9, IPTG was added to achieve a final concentration of 1 mM and the culture was continued with shaking for overnight at 30° C. Cells were then collected, lysed with polymyxin B (Sigma, St Louis) in PBS, and the supernatant was subjected to the Ni-NTA agarose bead (Qiagen) purification for the soluble scFvs. The various b12 scFv-Fc constructs as well as the germline b12 IgG construct were transfected into the 293 freestyle cells with polyfectin transfection agent (Invitrogen). Four days after transfection, the culture medium was collected and the secreted scFv-Fc and IgG proteins were purified using a protein-A sepharose column (GE Healthcare, Piscataway, N.J.)

ELISA

Different protein antigens were diluted in the PBS buffer in concentrations ranging from 1-4 μg/ml and coated to the 96 well plate at 4° C. for overnight. The plate was then blocked with PBS+5% dry milk buffer. Antibodies in various formats were diluted in the same blocking buffer and applied to the ELISA plate. The mouse-anti-His-HRP was used to detect the His tag at the C terminus end of each of the scFvs in most of the ELISA and the mouse-anti-human Fc-HRP was used to detect the scFv and IgG bindings. ABTS was then added to each well and OD 405 was taken 5-10 minutes afterward.

Pseudovirus Neutralization Assay

HIV Env pseudotyped virus preparation and neutralization was performed as previously described (Choudhry et al., 2007).

### Results

Binding of Mature and Germline-Like scFv X5 to Env—Dominant Role of the Heavy Chain

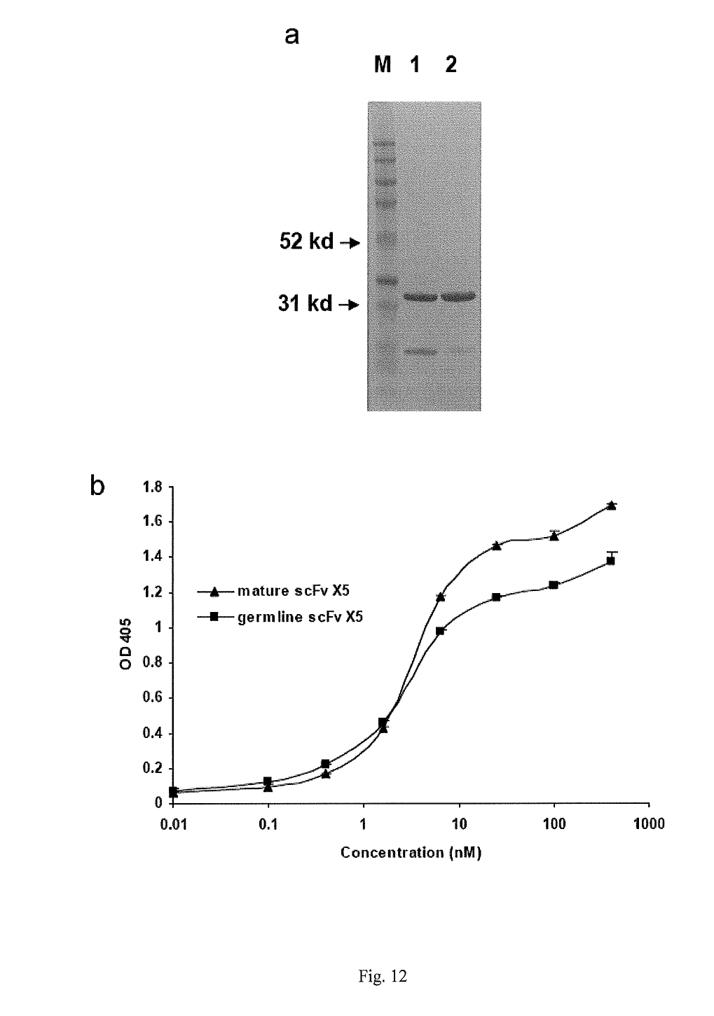

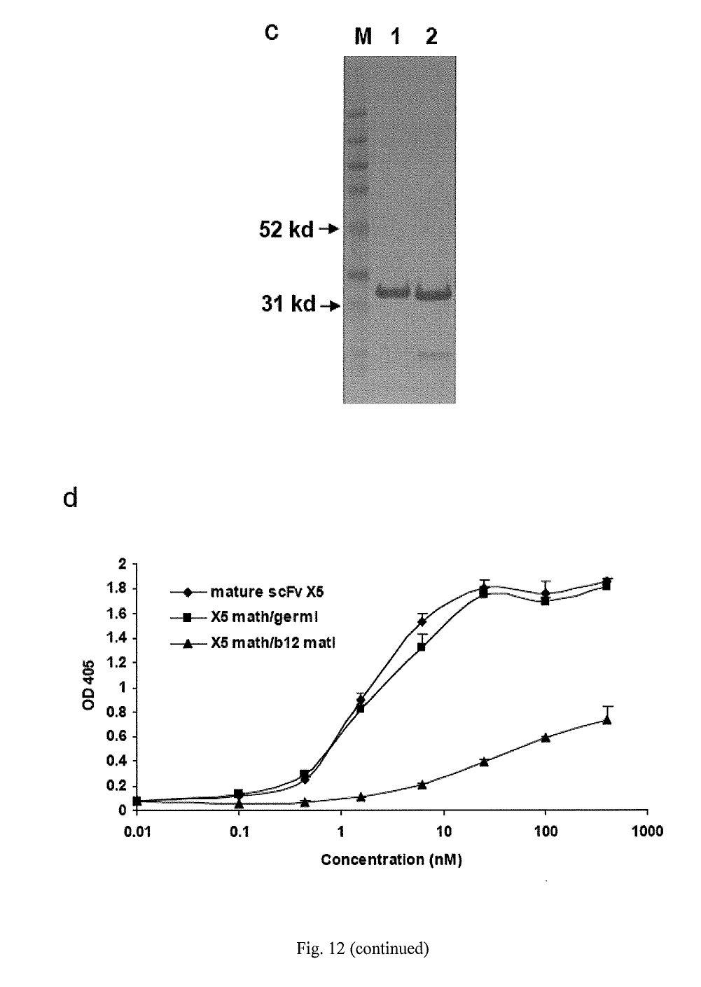

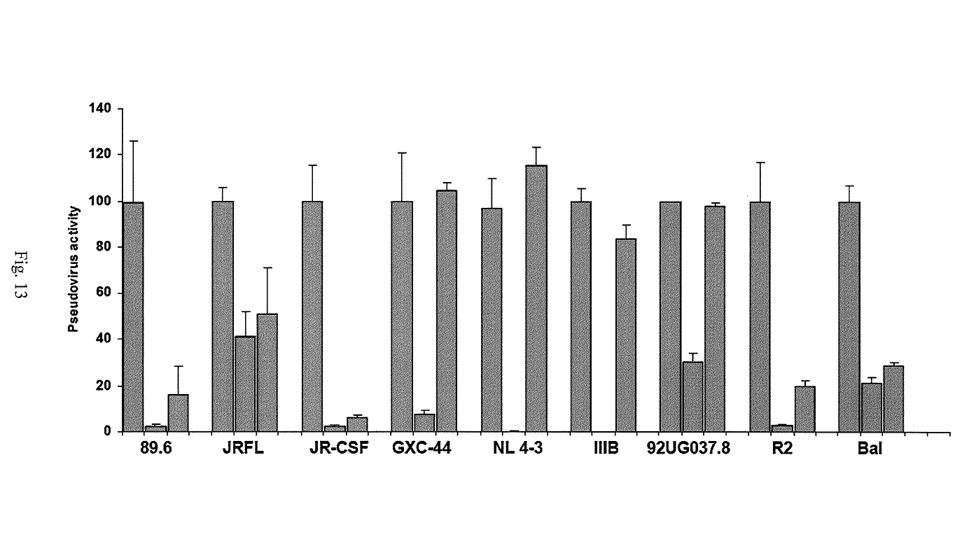

It has previously been found above that germline-like X5 binds to Env with relatively high strength (low EC50) which is only slightly lower than that for the mature antibody (FIG. 12 and Example 9). To further characterize this interaction and explore the relative contributions of the heavy and light chains to the interaction, hybrid molecules containing the heavy chain of X5 combined with the light chain of the germline-like X5 were generated, expressed and purified (FIG. 12c). The hybrid between the mature X5 heavy chain and its germline-like light chain bound better than did the germline X5 but similarly to the mature X5, underlying the dominant role of the heavy chain in determining the binding specificity and affinity (FIG. 12d).

Another hybrid between mature X5 heavy chain and mature b12 light chain was generated. This hybrid bound weaker than the germline X5 (FIG. 12d). In all cases the hybrid molecules bound the Env suggesting that the heavy chain of the mature X5 and likely of the germline-like X5 dominates the interaction (FIG. 12).

Germline-Like X5 Neutralized a Subset of Pseudoviruses Neutralized by the Mature X5

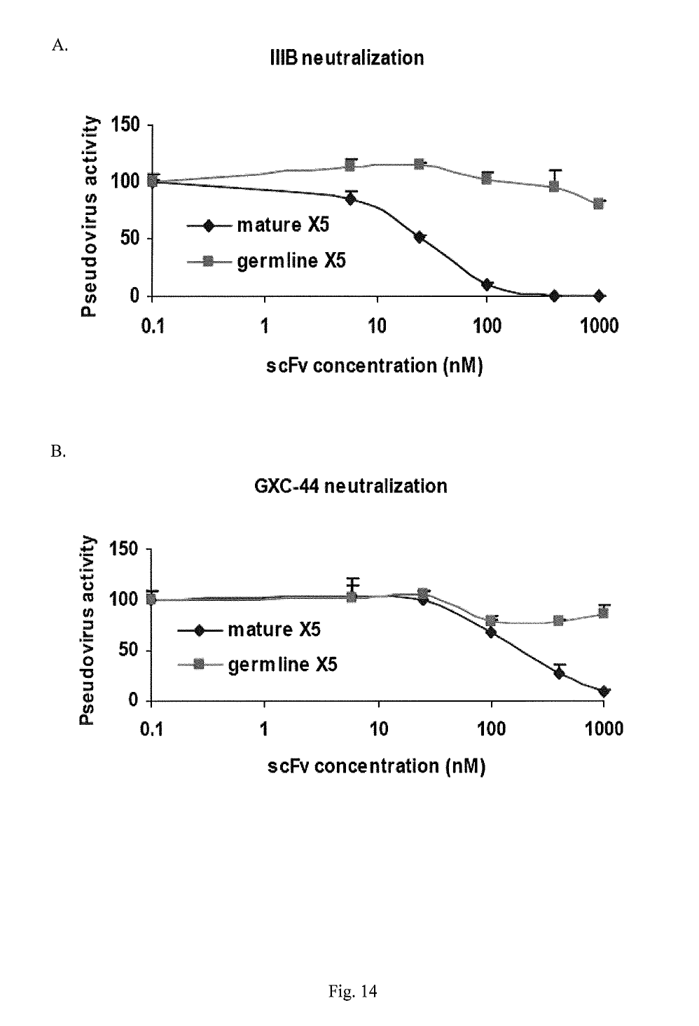

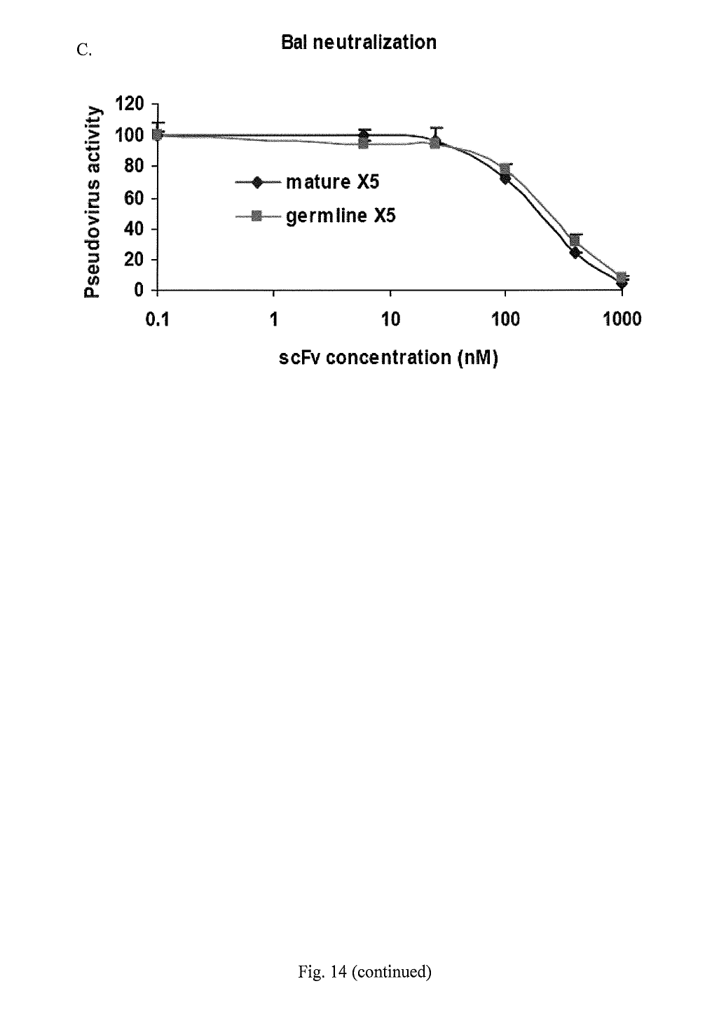

To test the neutralizing activity of germline-like scFv X5 relative to the mature antibody, a panel of pseudoviruses with Envs from isolates from Clades A, B, and C were used. The mature X5 neutralized all of them efficiently at the concentration used. The germline X5 neutralized all B Glade, R5 or dual tropic isolates almost as efficiently as the mature one, but lost completely the neutralizing ability against B Glade X4 tropic isolates as well as isolates from other clades (FIG. 13). To confirm this observation, dose response curves were constructed for both mature and germline-like X5 against three representative isolates (FIG. 14). While the mature and germline-like X5 exhibited similar IC50s against Bal pseudovirus, there was complete lack of neutralization by the germline-like X5 against IIIB and GXC-44. These data, although based on a limited number of isolates tested, indicate a possible mechanism of how X5 could have evolved from a Glade and tropism specific neutralizing antibody to a relatively broadly neutralizing antibody by acquiring somatic mutations. However, the X5 activity is measured for X5 in a scFv format. As discussed above full-size X5 is less potent than scFv X5 and its maturation pathway in vivo is likely to be complex.

Lack of Measurable Binding of Germline-Like b12 as scFv and as a Bivalent Fc Fusion Protein to a Panel of Envs

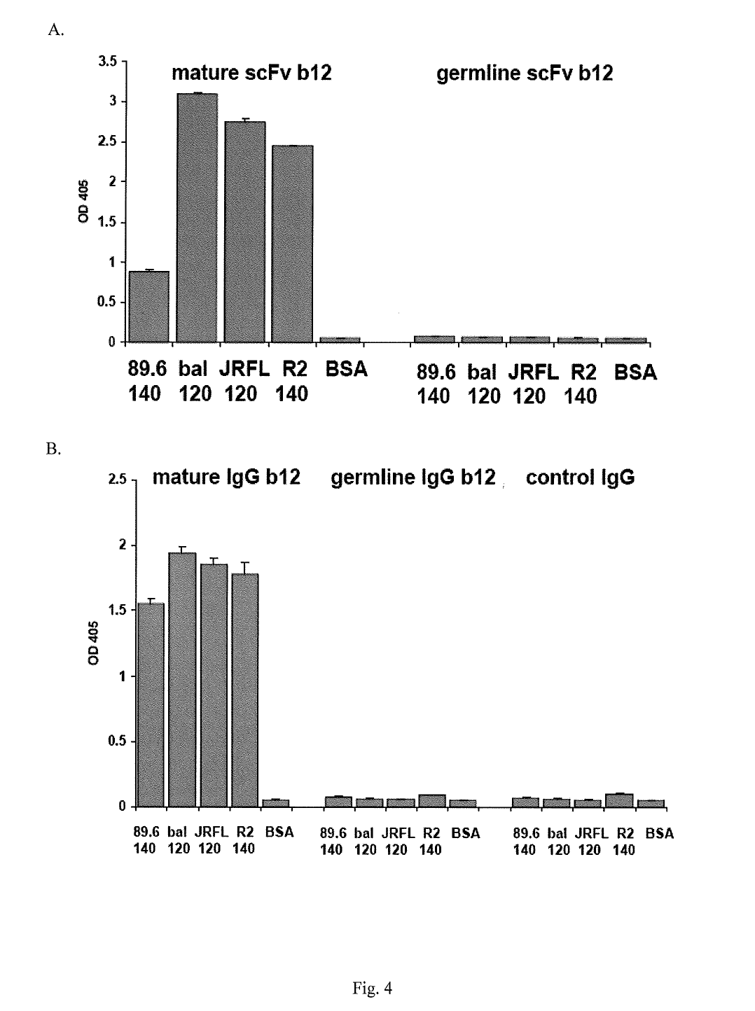

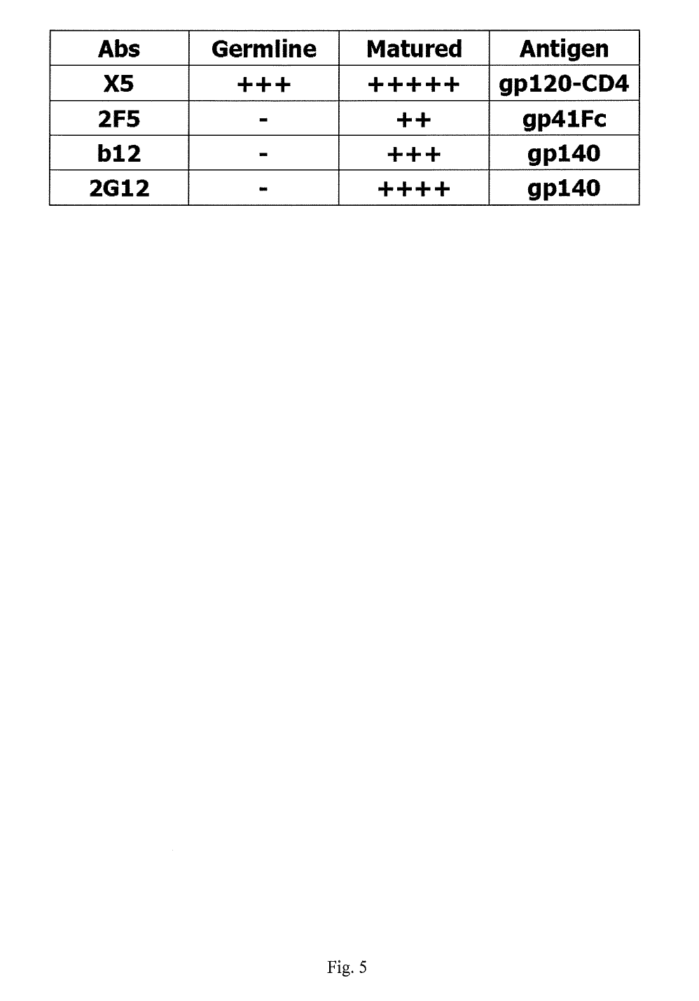

It has previously been found that germline-like b12 lacks measurable binding to an Env in the ELISA assay of Example 9. This observation is extended and confirmed using a panel of Envs. In all cases we found that germline-like b12 in both scFv and IgG formats lacks measurable binding to this panel of Envs (FIG. 4). In contrast, as expected the mature b12 bound strongly to all tested Envs (FIG. 4).

Identification of Possible Intermediates in the Maturation Pathway of b12

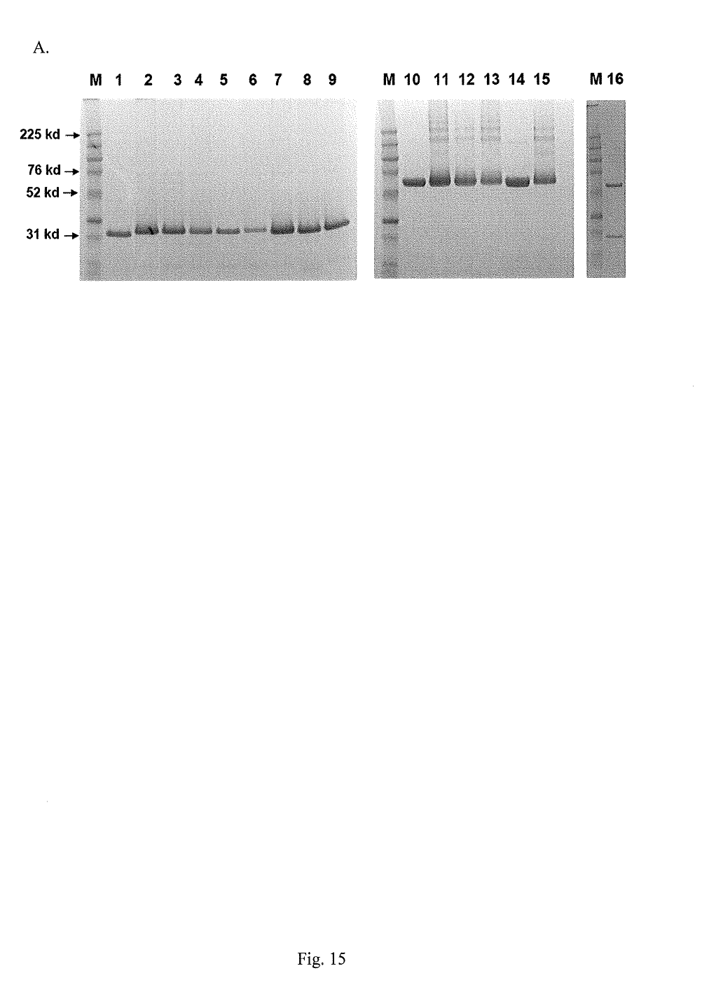

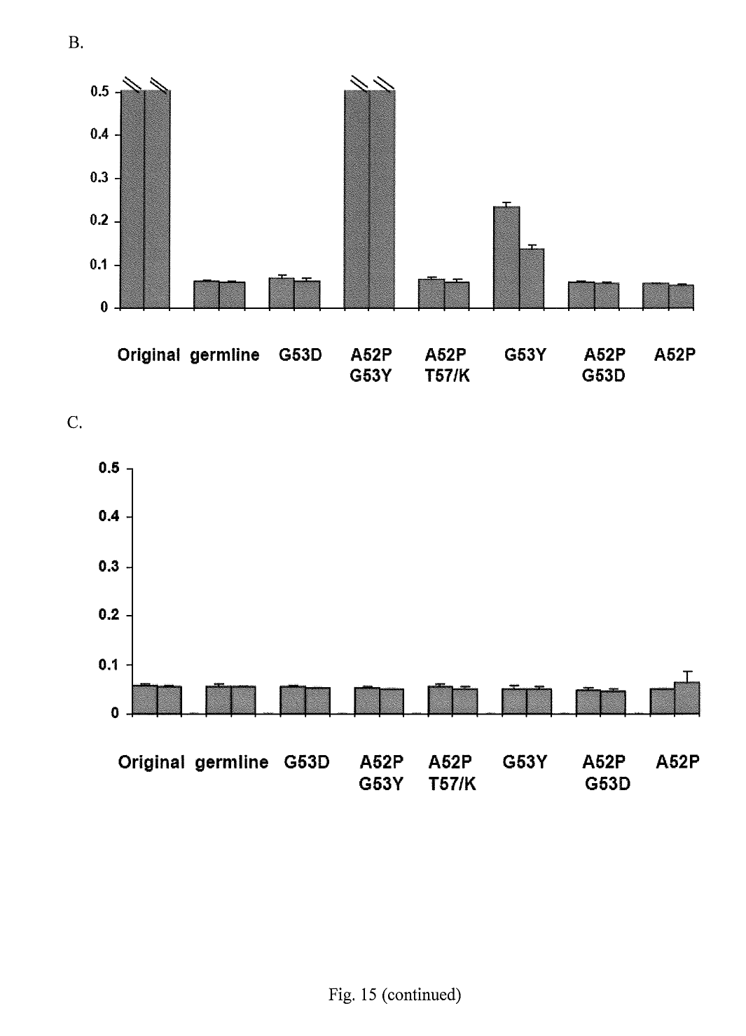

Intermediates in maturation pathway of b12 were sought by introducing critical residues found in mature b12 back into the germline framework of b12. H2 was used as the starting point for several reasons. First, the heavy chain is likely the major determinant for binding as indicated by the crystal structure of b12 in complex with gp120. Secondly, most of the amino acid substitutions in H1(4 of 5) between germline and mature forms are similar in nature. In contrast, all three mutations in H2 of the mature b 12 resulted in amino acids that are very different from their germline counterparts. A series of mutants were generated surrounding the germline b12 H2 region as described above in the Methods. In order to prevent potential poor expression or folding of certain mutants from distorting the data interpretation, all the mutants investigated were expressed and purified to homogeneity both as scFv and scFv-Fc soluble fusion proteins (FIG. 15a). The b12 germline-like antibdy was also expressed in the IgG format (FIG. 15a). In an initial screening with one single high antibody concentration, a single mutation G53Y was found to be sufficient to confer binding ability to germline b12. Additional mutations, such as A52P, increased the binding ability significantly (FIG. 15b).

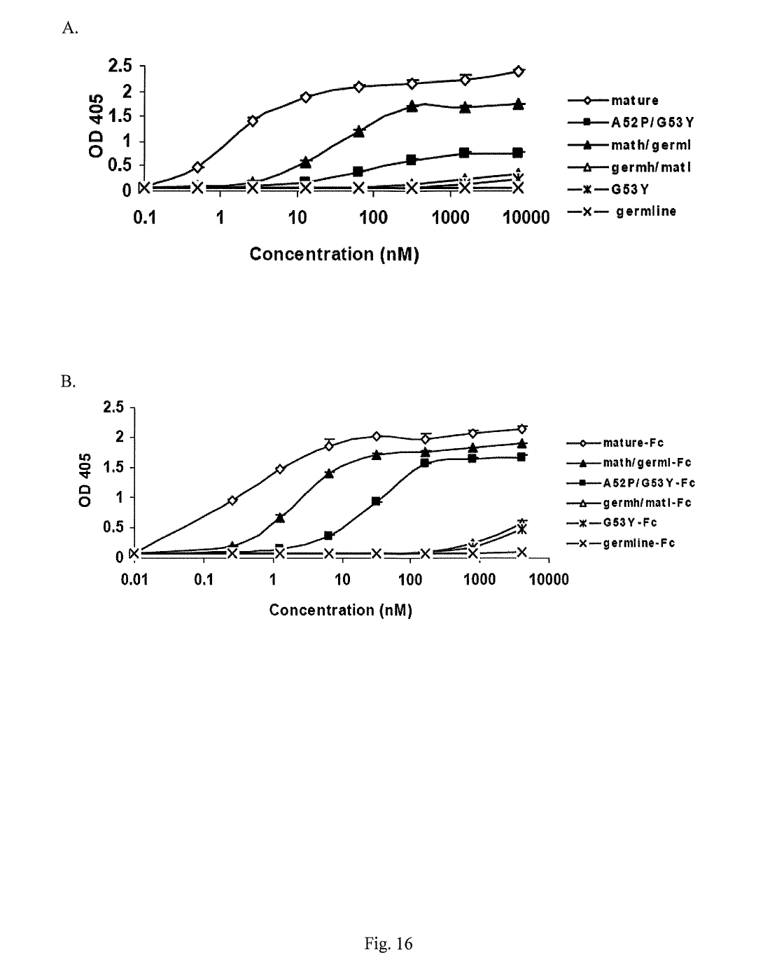

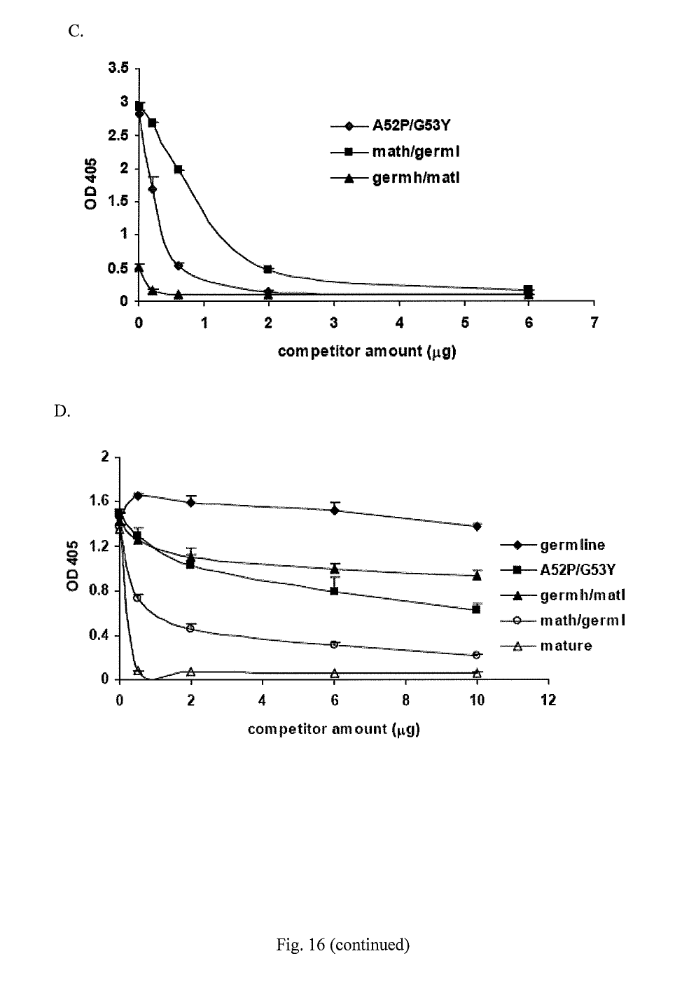

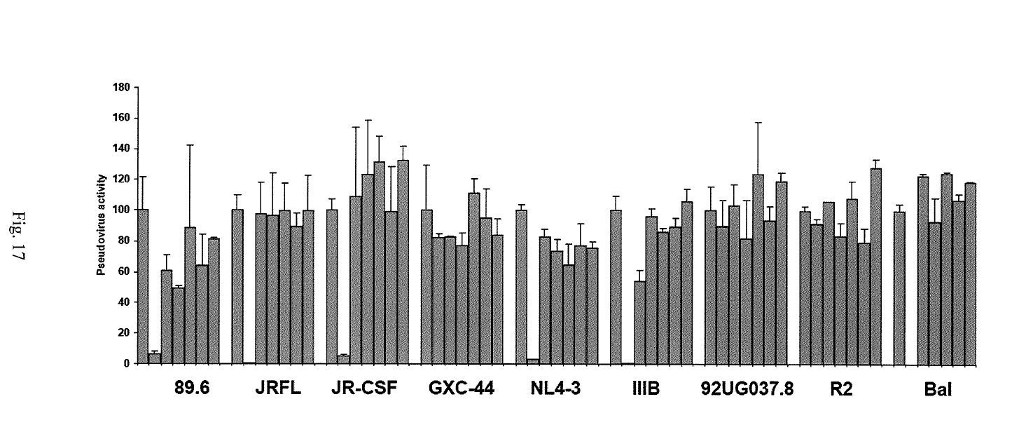

To confirm the data obtained in the initial screening with a single high concentration, an ELISA was performed using a range of concentrations of various scFv and scFv-Fc b12. The binding by the germline mutant G53Y was consistently detectable (FIGS. 16 a,b). A hybrid between mature b12 light chain and germline b12 heavy chain also displayed binding ability, albeit weak (FIGS. 16 a,b). The avidity effect was evident when the bindings by scFv and corresponding scFv-Fc were compared (FIGS. 16 a,b). b12 germline consistently showed no binding even at the highest concentration.

Two more layers of specificity control were used in addition to BSA, which was used as a control antigen in all experiments. First, it was found that all the weak bindings detected by the b12 intermediates can be completely competed out by the mature b12 (FIG. 16c). Further, all the b12 intermediates competed with sCD4 for binding to gp120, and the competition was proportional to their binding abilities. Germline b12, on the other hand, did not show any competition with sCD4 (FIG. 6d). These results indicate that the two mutations, G53Y and A52P, could play a role in the pathway from germline b12 to mature b12. See Table 6.

TABLE 6
Summary of the binding characteristics of the mature, germline and
intermediate scFv and scFv-Fc b12 as determined by ELISA. The antigen is bal gp120.

| b12 | | | A52P/ | | A52P/ | germh/ | math/ | |
|---|---|---|---|---|---|---|---|---|
| construct | germline | A52P | T57K | G53Y | G53Y | matl | germl | mature |
| scFv | DB | DB | DB | >μM | 87.3 nM | >μM | 34.5 nM | 1.3 nM |
| scFv-Fc | DB | N/A | N/A | >μM | 29.3 nM | >μM | 2.6 nM | 0.4 nM |

DB, did not bind;
N/A, did not test;
“>μM”, binding affinity in the range above μM.
Germh/matl, germline heavy chain fused with mature light chain.
Math/germl, mature heavy chain fused with germline light chain.

Inhibition of Pseudovirus Infection by B12 Intermediates

The neutralizing abilities of mature, germline and various intermediate b12s in their scFv-Fc format were tested against a panel of HIV Env pseudotyped viruses. The mature b12-Fc neutralized efficiently all isolates from Glade B except R2, which is a CD4 independent isolate. The mature b12-Fc also failed to inhibit two isolates from Glade A and C including isolates GXC-44 and 92UG037.8. This is in agreement with previous findings that b12 is most efficient against B Glade isolates. None of the b12 intermediates displayed significant neutralizing ability with the exception of the hybrid mature heavy chain/germline light chain (math/germl), which has relatively high binding affinity (34.5 nM) but neutralized IIIB with modest activity at a concentration significantly higher than the concentration needed for 50% binding (FIG. 17). These results indicate that some potential b12 intermediate antibodies may not exert selection pressure for generation of HIV-1 mutants.

Binding of Germline-Like and Intermediate b12 Antibodies to Human Cell Lines

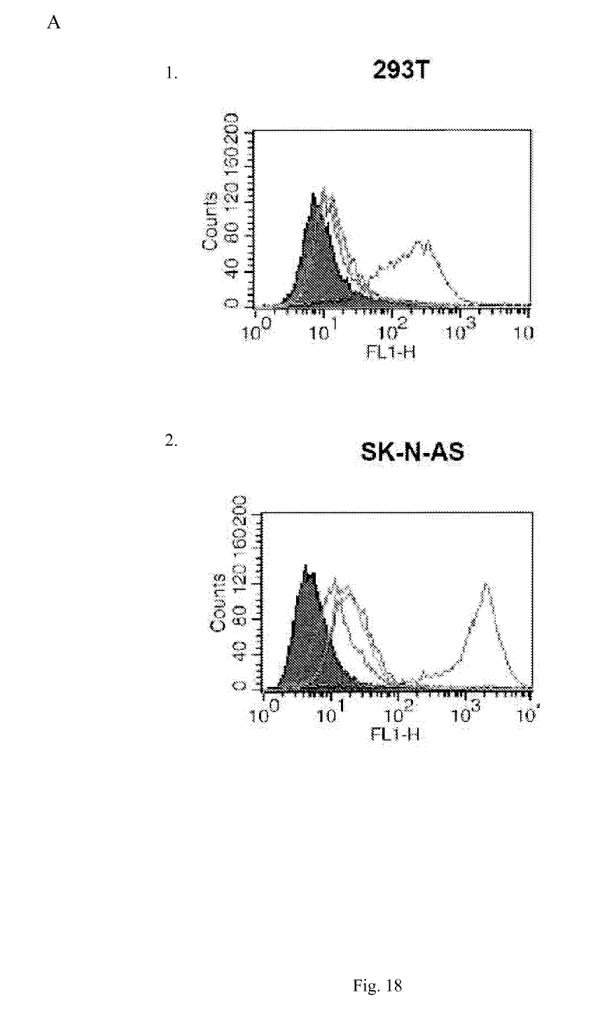

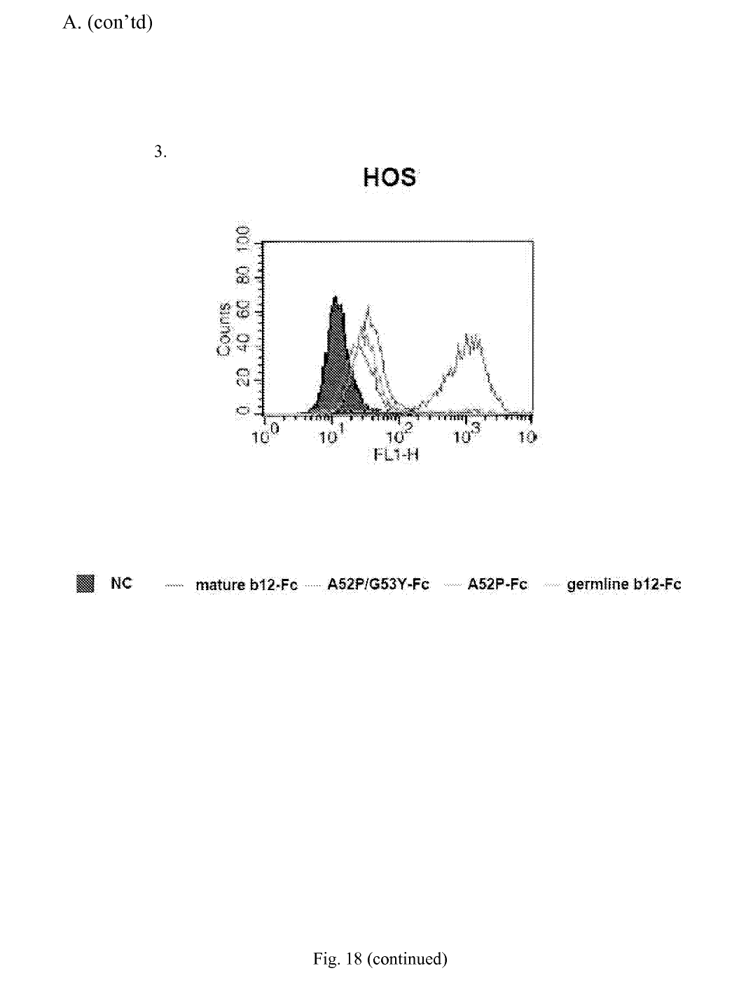

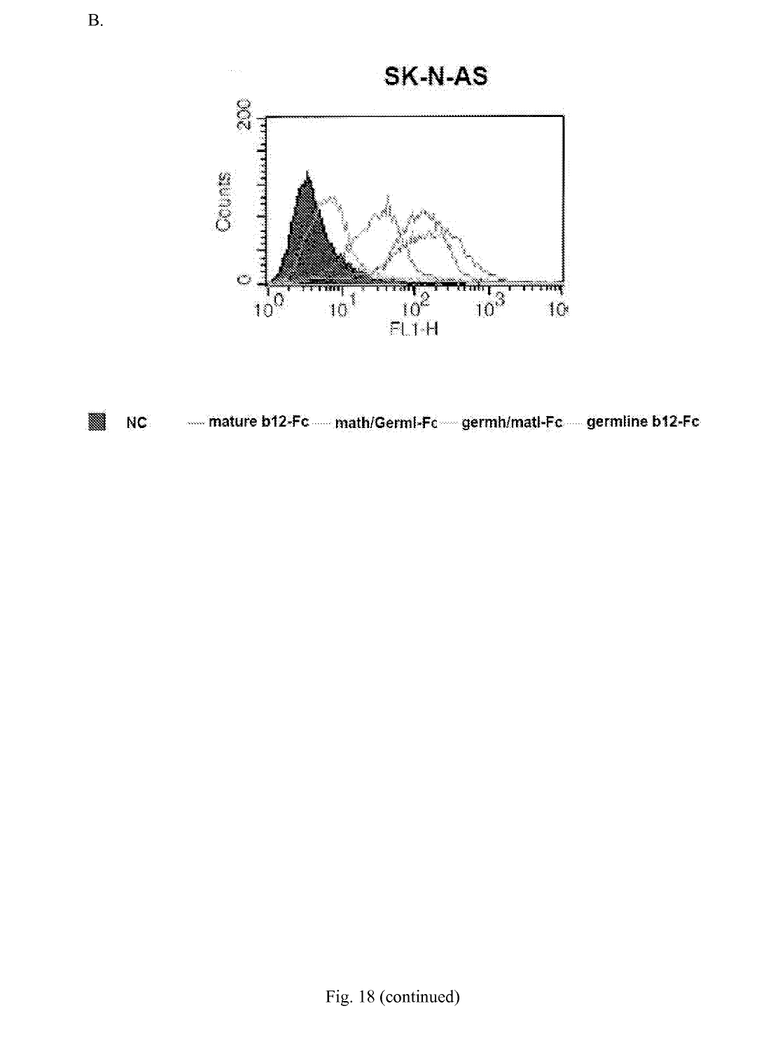

The accumulating numbers of somatic mutations during the b12 maturation contributed to the incremental increase in its binding to Envs. To test how various b12 somatic mutations contribute to self antigen bindings, three human cells lines were used in a flow cytometry analysis. The data show that the mature b12 binds strongly to these cells (FIG. 18). The germline-like b12 and intermediate b12 with small number of mutations (A52P/G53Y and A52/P) displayed much lower although measurable activities to these cell lines (FIG. 18). When the intermediates with large number of mutations, such as hybrid math/germl and germh/matl antibodies were tested, they showed significantly higher human cell binding approaching the level displayed by the mature b12. These findings indicate that specific binding to Env and self antigens were probably acquired concomitantly through the b12 maturation process.

### Discussion

Extensive somatic mutations have been found in all identified broadly neutralizing HIV antibodies and in most other HIV specific antibodies. This is in contrast to some of the potent neutralizing antibodies against acute infections (see above in Example 9). These antibodies possess few if any mutations compared to their germline sequences. Knowledge of whether germline-like antibodies, corresponding to the HIV-1-neutralizing antibodies, possess neutralizing activity and how the somatic mutations can contribute to their binding and neutralizing function is limited. To better understand the antibody maturation pathways, the binding and neutralizing abilities of mature and germline forms of two HIV-1 neutralizing antibodies, X5 and b12 were analyzed.

IgG1 X5 is a modestly neutralzing antibody targeting a highly conserved CD4i epitope. Its corresponding germline antibody in a scFv format displayed high affinity to the Env tested in this study and neutralized efficiently several isolates. All these neutralized isolates belong to B clade and are either R5 tropic or dual tropic. On the other hand, the mature scFv X5 neutralized all isolates tested. These isolates are from A, B and C clades. These data suggest first that germline antibodies against certain epitopes on the Env, similarly to antibodies against other acute infections, do possess neutralizing ability. Secondly, the mutational process shifted or expanded the antibody binding epitope so that it became more inclusive leading to a more broadly neutralizing antibody. This notion is supported by a previous observation that a synthetic HIV-1 inhibitor based on CH2, m1a1, has an epitope that partially overlaps with that of germline X5 as revealed by competition ELISA. M1a1 has a tendency to neutralize only B clades, X4 tropic viruses in contrast to germline X5. The epitope of m1a1 overlaps significantly more with that of the mature X5 than with germline X5 as also revealed by competition ELISA.

Based on these data, it can be speculated that X5 originated from a germline antibody recognizing a B Glade, R5 tropic isolate. The subsequent mutations expanded its targets to include B Glade, X4 tropic isolates and those from other clades. X5 seems to follow a typical antibody maturation pathway, and this might explain partially the predominant presence of CD4i antibodies in HIV patients due to the fact that this epitope appears to be readily available for germline antibody recognition. It remains to be seen if germline counterparts of other CD4i antibodies possess antigen binding and neutralizing abilities as observed with X5.

In contrast to X5 b12 appears to follow a different pathway. The germline b12 lacks observable binding to a panel of Envs confirming and expanding the observation of a lack of measurable binding of germline-like b12 to Env (Example 9). By systemic mutation of amino acid residues in the mature b12 into the corresponding locations on germline b12, several possible intermediates at different stages along the maturation pathway of b12 were identified. Importantly, the increase in binding against the Env associated with the increasing number of somatic mutations in these intermediates seems to be closely related to their increase in binding to human antigens.

These data reveal a possible interplay between the Env and human self antigens in the origination and maturation of b 12. One or more alternative antigens, self-antigens included, were likely responsible for the initial activation of the B cells expressing b12 germline like antibodies. The somatic mutations ensued after the activation may have enabled b12 intermediate(s) to bind to other antigens including Envs. The fact that a single mutation, G53Y, conferred detectable germline-like b12 binding to Env as found in this Example indicates the possibility of this scenario. These intermediate antibodies are currently used as reagents to identify molecules that could serve as primary immunogens for initiation of the maturation of b12 or b12-like antibodies. The primary immunogens to be found could be used in combination with appropriately designed Envs exposing the b12 epitopes and lacking other immunodominant epitopes. This conceptually new two (or more) immunogen approach for guiding the immune system through the complex maturation pathways of known antibodies with high activity is more general and could be used to help design of vaccine immunogens also for other diseases including cancer.

Interestingly, the data also revealed some unexpected molecular features of the light chains. First, the hybrid between mature b12 heavy chain and germline like b12 showed efficient bindings to Envs tested. This is surprising given previous findings that single mutations targeting R residues within the b12 μl essentially eliminated b12 binding ability (Zwick et al., 2003). These R residues are completely lacking in the germline like b12 light chain. Secondly, the hybrid between germline-like b12 heavy chain and mature b12 light chain displayed consistently specific binding. This seems to suggest that b12 light chain in its mature form can form binding paratopes independent of the heavy chain, even though the possibility cannot be ruled out that matured b12 light chain can assist the heavy chain in its germline-like form to bind. Finally, even though all the intermediates, in particular the math/germl bound HIV Env, none possessed the neutralizing ability reflecting that of matured b12. This can not be simply explained by affinity alone due to the very high concentrations of antibodies used in neutralization assay.

In summary, several human monoclonal antibodies (hmAbs) and antibody fragments, including the best characterized in terms of structure-function b12 and Fab X5, exhibit relatively potent and broad HIV-1 neutralizing activity. However, the elicitation of b12 or b12-like antibodies in vivo by vaccine immunogens based on the HIV-1 envelope glycoprotein (Env) has not been successful. B12 is highly divergent from the closest corresponding germline antibody while X5 is less divergent. The relatively high degree of specific somatic hypermutations may preclude binding of the HIV-1 envelope glycoprotein (Env) to closest germline antibodies, and that identifying antibodies that are intermediates in the pathways to maturation could help design novel vaccine immunogens to guide the immune system for their enhanced elicitation. It was found that a germline-like b12 (monovalent and bivalent scFv as an Fc fusion protein or IgG) lacks measurable binding to an Env as measured by ELISA with a sensitivity in the μM range (see Example 9). This Example presents evidence confirming and expanding these findings for a panel of Envs. In contrast, a germline-like scFv X5 bound Env with high (nM) affinity. To begin to explore the maturation pathways of these antibodies, several possible b12 intermediate antibodies were designed and their neutralizing activity was tested. These intermediate antibodies neutralized only some HIV-1 isolates and with relatively weak potency. In contrast, germline-like scFv X5 neutralized most of the tested HIV-1 isolates with comparable efficiencies to that of the mature X5. These results could help explain the relatively high immunogenicity of the coreceptor binding site on gp120 and the abundance of CD4-induced (CD4i) antibodies in HIV-1-infected patients (X5 is a CD4i antibody) as well as the maturation pathway of X5. They also can help identify antigens that can bind specifically to b12 germline and intermediate antibodies that together with Envs could be used as a conceptually novel type of candidate vaccines. Such candidate vaccines based on two or more immunogens could help guide the immune system through complex maturation pathways for elicitation of antibodies that are similar or identical to antibodies with known properties.

### REFERENCES

The following references, which are cited in the above paragraphs, are hereby incorporated by reference.
Alam, S. Munir, McAdams, M., Boren, D., Rak, M., Scearce, R. M., Gao, F., Camacho, Z. T., Gewirth, D., Kelsoe, G., Chen, P., Haynes, B. F. The Role of Antibody Polyspecificity and Lipid Reactivity in Binding of Broadly Neutralizing anti-HIV-1 Envelope Human Monoclonal Antibodies 2F5 and 4E10 to gp41 Membrane Proximal Envelope Epitopes. J. Immunol., 178:4424-4435, 2007.
Andris, J. S., Johnson, S., Zolla-Pazner, S., Capra, J. D., 1991. Molecular characterization of five human antihuman immunodeficiency virus type 1 antibody heavy chains reveals extensive somatic mutation typical of an antigen-driven immune response. Proc. Natl. Acad. Sci. U.S.A. 88, 7783-7787.
Binley, J. M., Ditzel, H. J., Barbas, C. F. 3., Sullivan, N., Sodroski, J., Parren, P. W., Burton, D. R., 1996. Human antibody responses to HIV type 1 glycoprotein 41 cloned in phage display libraries suggest three major epitopes are recognized and give evidence for conserved antibody motifs in antigen binding. AIDS Res. Hum. Retroviruses 12, 911-924. Binley, J. M., Wrin, T., Korber, B., Zwick, M. B., Wang, M., Chappey, C., Stiegler, G., Kunert, R., Zolla-Pazner, S., Katinger, H., Petropoulos, C. J., Burton, D. R., Comprehensive cross-clade neutralization analysis of a panel of anti-human immunodeficiency virus type 1 monoclonal antibodies J. Virol. 78, (2004) 13232-13252
Burton, D. R., 2002. Antibodies, viruses and vaccines. Nat. Rev. Immunol. 2, 706-713.
Burton, D. R., Montefiori, D. C., 1997. The antibody response in HIV-1 infection. AIDS 11 Suppl A, S87-S98.
Burton, D. R., Pyati, J., Koduri, R., Sharp, S. J., Thornton, G. B., Parren, P. W., Sawyer, L. S., Hendry, R. M., Dunlop, N., Nara, P. L., et al., 1994. Efficient neutralization of primary isolates of HIV-1 by a recombinant human monoclonal antibody. Science 266, 1024-1027.
Chapal, N., Bouanani, M., Embleton, M. J., Navarro-Teulon, I., Biard-Piechaczyk, M., Pau, B., Peraldi-Roux, S., 1997. In-cell assembly of scFv from human thyroid-infiltrating B cells. Biotechniques 23, 518-524.
Chapal, N., Chardes, T., Bresson, D., Pugniere, M., Mani, J. C., Pau, B., Bouanani, M., Peraldi-Roux, S., 2001. Thyroid peroxidase autoantibodies obtained from random single chain FV libraries contain the same heavy/light chain combinations as occur in vivo. Endocrinology 142, 4740-4750.
Chen, W, Z Zhu, Z., Zhang, M, Macagno, A, Prabakaran, P., Owens, J., Longo, N. S., Markowitz, M., Lanzavecchia, A., Haynes, B. F., Dimitrov, D. S. All known cross reactive HIV-1 neutralizing antibodies are highly divergent from germline and their elicitation may require prolonged periods of time AIDS Res Hum Retroviruses 24(Suppl. 1), (2008) 11-12.
Choudhry, V., Zhang, M. Y., Dimitrova, D., Prabakaran, P., Dimitrov, A. S., Fouts, T. R., Dimitrov, D. S., 2006. Antibody-based inhibitors of HIV infection. Expert. Opin. Biol. Ther. 6, 523-531.
Choudhry, V., Zhang, M. Y., Sidorov, I. A., Louis, J. M., Harris, I., Dimitrov, A. S., Bouma, P., Cham, F., Choudhary, A., Rybak, S. M., Fouts, T., Montefiori, D. C., Broder, C. C., Quinnan, G. V., Jr., Dimitrov, D. S., 2007. Cross-reactive HIV-1 neutralizing monoclonal antibodies selected by screening of an immune human phage library against an envelope glycoprotein (gp140) isolated from a patient (R2) with broadly HIV-1 neutralizing antibodies. Virology 363, 79-90.
Clark, L. A., Ganesan, S., Papp, S., van Vlijmen, H. W., 2006. Trends in antibody sequence changes during the somatic hypermutation process. J. Immunol. 177, 333-340.
Costante, G., Portolano, S., Nishikawa, T., Jaume, J. C., Chazenbalk, G. D., Rapoport, B., McLachlan, S. M., 1994. Recombinant thyroid peroxidase-specific autoantibodies. II. Role of individual heavy and light chains in determining epitope recognition. Endocrinology 135, 25-30.
Dal Porto, J. M., Haberman, A. M., Kelsoe, G., Shlomchik, M. J., Very low affinity B cells form germinal centers, become memory B cells, and participate in secondary immune responses when higher affinity competition is reduced, J. Exp. Med. 195 (2002) 1215-1221.
Dal Porto, J. M., Haberman, A. M., Shlomchik, M. J., Kelsoe, G., Antigen drives very low affinity B cells to become plasmacytes and enter germinal centers, J. 473 Immunol. 161 (1998) 5373-5381.
Decker, J. M., Bibollet-Ruche, F., Wei, X., Wang, S., Levy, D. N., Wang, W., Delaporte, E., Peeters, M., Derdeyn, C. A., Allen, S., Hunter, E., Saag, M. S., Hoxie, J. A., Hahn, B. H., Kwong, P. D., Robinson, J. E.,
Shaw, G. M., 2005. Antigenic conservation and immunogenicity of the HIV coreceptor binding site. J. Exp. Med. 201, 1407-1419.
Felgenhauer, M., Kohl, J., Ruker, F., 1990. Nucleotide sequences of the cDNAs encoding the V-regions of H- and L-chains of a human monoclonal antibody specific to HIV-1-gp41. Nucleic Acids Res. 18, 4927.
Ferrantelli, F., Ruprecht, R. M., 2002. Neutralizing antibodies against HIV—back in the major leagues? Curr. Opin. Immunol. 14, 495-502.
Garber, D. A., Silvestri, G., Feinberg, M. B., 2004. Prospects for an AIDS vaccine: three big questions, no easy answers. Lancet Infect. Dis. 4, 397-413.
Gorny, M. K., Conley, A. J., Karwowska, S., Buchbinder, A., Xu, J. Y., Emini, E. A., Koenig, S., Zolla-Pazner, S., 1992. Neutralization of diverse human immunodeficiency virus type 1 variants by an anti-V3 human monoclonal antibody. J. Virol. 66, 7538-7542.
Haynes, B. F., Fleming, J., St Clair, W. E., Katinger, H., Stiegler, G., Kunert, R., Robinson, J., Scearce, R. M., Plonk, K., Staats, H. F., Ortel, T. L., Liao, H. X., Alam, M. S., 2005a. Cardiolipin Polyspecific Autoreactivity in Two Broadly Neutralizing HIV-1 Antibodies. Science.
Haynes, B. F., Montefiori, D. C., 2006. Aiming to induce broadly reactive neutralizing antibody responses with HIV-1 vaccine candidates. Expert. Rev. Vaccines. 5, 579-595.
Haynes, B. F., Moody, M. A., Verkoczy, L., Kelsoe, G., Alam, S. M., 2005b. Antibody polyspecificity and neutralization of HIV-1: a hypothesis. Hum. Antibodies 14, 59-67.
Huang, C. C., Venturi, M., Majeed, S., Moore, M. J., Phogat, S., Zhang, M. Y., Dimitrov, D. S., Hendrickson, W. A., Robinson, J., Sodroski, J., Wyatt, R., Choe, H., Farzan, M., Kwong, P. D., 2004. Structural basis of tyrosine sulfation and VH-gene usage in antibodies that recognize the HIV type 1 coreceptor-binding site on gp120. Proc. Natl. Acad. Sci. U.S.A 101, 2706-2711.
Jaume, J. C., Costante, G., Portolano, S., McLachlan, S. M., Rapoport, B., 1994. Recombinant thyroid peroxidasespecific autoantibodies. I. How diverse is the pool of heavy and light chains in immunoglobulin gene libraries constructed from thyroid tissue-infiltrating plasma cells. Endocrinology 135, 16-24.
Johnson, W. E., Desrosiers, R. C., 2002. Viral persistance: HIV's strategies of immune system evasion. Annu Rev. Med. 53, 499-518.
Kalinke, U., Oxenius, A., Lopez-Macias, C., Zinkemagel, R. M., Hengartner, H. Virus neutralization by germ-line vs. hypermutated antibodies Proc. Natl. Acad. Sci U.S.A 97, (2000) 10126-10131.
Koelsch, K., Zheng, N. Y., Zhang, Q., Duty, A., Helms, C., Mathias, M. D., Jared, M., Smith, K., Capra, J. D., Wilson, P. C., 2007. Mature B cells class switched to IgD are autoreactive in healthy individuals. J. Clin. Invest 117, 1558-1565.
Labrijn, A. F., Poignard, P., Raja, A., Zwick, M. B., Delgado, K., Franti, M., Binley, J., Vivona, V., Grundner, C., Huang, C. C., Venturi, M., Petropoulos, C. J., Wrin, T., Dimitrov, D. S., Robinson, J., Kwong, P. D., Wyatt, R. T., Sodroski, J., Burton, D. R., 2003. Access of antibody molecules to the conserved coreceptor binding site on glycoprotein gp120 is sterically restricted on primary human immunodeficiency virus type 1. J. Virol. 77, 1055710565.
Longo, N. S., Lugar, P. L., Yavuz, S., Zhang, W., Krijger, P. H., Russ, D. E., Jima, D. D., Dave, S. S., Grammer, A. C., Lipsky, P. E., Analysis of somatic hypermutation in X-linked hyper-IgM syndrome shows specific deficiencies in mutational targeting, Blood 113 (2009) 3706-3715.
Louis, J. M., Bewley, C. A., Gustchina, E., Aniana, A., Clore, G. M., 2005. Characterization and HIV-1 fusion inhibitory properties of monoclonal Fabs obtained from a human non-immune phage library selected against diverse epitopes of the ectodomain of HIV-1 gp41. J Mol. Biol. 353, 945-951.
Marasco, W. A., Bagley, J., Zani, C., Posner, M., Cavacini, L., Haseltine, W. A., Sodroski, J., 1992. Characterization of the cDNA of a broadly reactive neutralizing human anti-gp120 monoclonal antibody. J. Clin. Invest 90, 1467-1478.
Max, E. E., 2003. Immunoglobulins: Molecular Genetics. In: W. E. Paul (Ed.), Fundamental Immunology. Lippincott Williams & Wilkins, Philadelphia, pp. 107-158.
Meijer, P. J., Andersen, P. S., Haahr, H. M., Steinaa, L., Jensen, A., Lantto, J., Oleksiewicz, M. B., Tengbjerg, K., Poulsen, T. R., Coljee, V. W., Bregenholt, S., Haurum, J. S., Nielsen, L. S., 2006. Isolation of human antibody repertoires with preservation of the natural heavy and light chain pairing. J. Mol. Biol. 358, 764-772.
Miller, M. D., Geleziunas, R., Bianchi, E., Lennard, S., Hrin, R., Zhang, H., Lu, M., An, Z., Ingallinella, P., Finotto, M., Mattu, M., Finnefrock, A. C., Bramhill, D., Cook, J., Eckert, D. M., Hampton, R., Patel, M., Jarantow, S., Joyce, J., Ciliberto, G., Cortese, R., Lu, P., Strohl, W., Schleif, W., McElhaugh, M., Lane, S., Lloyd, C., Lowe, D., Osbourn, J., Vaughan, T., Emini, E., Barbato, G., Kim, P. S., Hazuda,
D. J., Shiver, J. W., Pessi, A., 2005. A human monoclonal antibody neutralizes diverse HIV-1 isolates by binding a critical gp41 epitope. Proc Natl. Acad Sci U.S.A 102, 14759-14764.
Moran, M. J., Andris, J. S., Matsumato, Y., Capra, J. D., Hersh, E. M., 1993. Variable region genes of anti-HIV human monoclonal antibodies: non-restricted use of the V gene repertoire and extensive somatic mutation. Mol. Immunol. 30, 1543-1551.
Moulard, M., Phogat, S. K., Shu, Y., Labrijn, A. F., Xiao, X. D., Binley, J. M., Zhang, M. Y., Sidorov, I. A., Broder, C. C., Robinson, J., Parren, P. W. H. I., Burton, D. R., Dimitrov, D. S., 2002. Broadly cross-reactive HIV1-neutralizing human monoclonal Fab selected for binding to gp120-CD4-CCR5 complexes. Proceedings of the National Academy of Sciences of the United States of America 99, 6913-6918.
Moulard, M., Zhang, M. Y., Dimitrov, D. S., 2003. Novel HIV neutralizing antibodies selected from phage display libraries. In: G. Subramanian (Ed.), Biopharmaceutical antibodies. Kluwer, New York, pp. 105-117.
Muster, T., Steindl, F., Purtscher, M., Trkola, A., Klima, A., Himmler, G., Ruker, F., Katinger, H., 1993. A conserved neutralizing epitope on gp41 of human immunodeficiency virus type 1. J. Virol. 67, 6642-6647.
Parren, P. W., Fisicaro, P., Labrijn, A. F., Binley, J. M., Yang, W. P., Ditzel, H. J., Barbas, C. F., III, Burton, D. R., 1996. In vitro antigen challenge of human antibody libraries for vaccine evaluation: the human immunodeficiency virus type 1 envelope. J. Viol. 70, 9046-9050.
Poignard, P., Saphire, E. O., Parren, P. W., Burton, D. R., 2001. Gp120: biologic aspects of structural features. Annu Rev. Immunol. 19, 253-274.
Prabakaran, P., Gan, J., Feng, Y., Zhu, Z., Choudhry, V., Xiao, X., Ji, X., Dimitrov, D. S., 2006a. Structure of severe acute respiratory syndrome coronavirus receptor-binding domain complexed with neutralizing antibody. J. Biol. Chem. 281, 15829-15836.
Prabakaran, P., Gan, J., Wu, Y. Q., Zhang, M. Y., Dimitrov, D. S., Ji, X., 2006b. Structural Mimicry of CD4 by a Cross-reactive HIV-1 Neutralizing Antibody with CDR-H2 and H3Containing Unique Motifs. J. Mol. Biol. Prabakaran, P., Zhu, Z., Xiao, X., Biragyn, A., Dimitrov, A. S., Broder, C. C., Dimitrov, D. S, Potent human monoclonal antibodies against SARS CoV, Nipah and Hendra viruses, Expert Opin. Biol. Ther. 9 (2009) 355-368.
Richman, D. D., Wrin, T., Little, S. J., Petropoulos, C. J., 2003. Rapid evolution of the neutralizing antibody response to HIV type 1 infection. Proc. Natl. Acad. Sci. U.S.A 100, 4144-4149.
Roben, P., Moore, J. P., Thali, M., Sodroski, J., Barbas, C. F. 3., Burton, D. R., 1994. Recognition properties of a panel of human recombinant Fab fragments to the CD4 binding site of gp120 that show differing abilities to neutralize human immunodeficiency virus type 1. J. Virol. 68, 4821-4828.
Sanders, R. W., Venturi, M., Schifiher, L., Kalyanaraman, R., Katinger, H., Lloyd, K. O., Kwong, P. D., Moore, J. P., 2002. The mannose-dependent epitope for neutralizing antibody 2G12 on human immunodeficiency virus type 1 glycoprotein gp120. J. Virol. 76, 7293-7305.
Scanlan, C. N., Pantophlet, R., Wormald, M. R., 011mann, S. E., Stanfield, R., Wilson, I. A., Katinger, H., Dwek, R. A., Rudd, P. M., Burton, D. R., 2002. The broadly neutralizing anti-human immunodeficiency virus type 1 antibody 2G12 recognizes a cluster of alpha1->2 mannose residues on the outer face of gp120. J. Virol. 76, 73067321.
Shih, T. A., Meffre, E., Roederer, M., Nussenzweig, M. C., Role of BCR affinity in T cell dependent antibody responses in vivo, Nat. Immunol. 3 (2002) 570-575.
Shih, T. A., Roederer, M., Nussenzweig, M. C., Role of antigen receptor affinity in T cell-independent antibody responses in vivo, Nat. Immunol. 3 (2002) 399-406.
Scott, M. G., Crimmins, D. L., McCourt, D. W., Chung, G., Schable, K. F., Thiebe, R., Quenzel, E. M., Zachau, H. G., Nahm, M. H., 1991. Clonal characterization of the human IgG antibody repertoire to Haemophilus influenzae type b polysaccharide. IV. The less frequently expressed VL are heterogeneous. J. Immunol. 147, 40074013.
Scott, M. G., Crimmins, D. L., McCourt, D. W., Zocher, I., Thiebe, R., Zachau, H. G., Nahm, M. H., 1989. Clonal characterization of the human IgG antibody repertoire to Haemophilus influenzae type b polysaccharide. HI. A single VKII gene and one of several JK genes are joined by an invariant arginine to form the most common L chain V region. J. Immunol. 143, 4110-4116.
Souto-Carneiro, M. M., Longo, N. S., Russ, D. E., Sun, H. W., Lipsky, P. E., Characterization of the human Ig heavy chain antigen binding complementarity determining region 3 using a newly developed software algorithm, JOINSOLVER, J. Immunol. 172 (2004) 6790-6802.
Stiegler, G., Kunert, R., Purtscher, M., Wolbank, S., Voglauer, R., Steindl, F., Katinger, H., 2001. A potent crossclade neutralizing human monoclonal antibody against a novel epitope on gp41 of human immunodeficiency virus type 1. AIDS Res. Hum. Retroviruses 17, 1757-1765.
Sui, J., L1, W., Murakami, A., Tamin, A., Matthews, L. J., Wong, S. K., Moore, M. J., Tallarico, A. S., Olurinde, M., Choe, H., Anderson, L. J., Bellini, W. J., Farzan, M., Marasco, W. A., 2004. Potent neutralization of severe acute respiratory syndrome (SARS) coronavirus by a human mAb to 51 protein that blocks receptor association. Proc Natl Acad Sci USA 101, 2536-2541.
Trkola, A., Purtscher, M., Muster, T., Ballaun, C., Buchacher, A., Sullivan, N., Srinivasan, K., Sodroski, J., Moore, J. P., Katinger, H., 1996. Human monoclonal antibody 2G12 defines a distinctive neutralization epitope on the gp120 glycoprotein of human immunodeficiency virus type 1. J. Virol. 70, 1100-1108.
Volkheimer, A. D., Weinberg, J. B., Beasley, B. E., Whitesides, J. F., Gockerman, J. P., Moore, J. O., Kelsoe, G., Goodman, B. K., Levesque, M. C., 2007. Progressive immunoglobulin gene mutations in chronic lymphocytic leukemia: evidence for antigen-driven intraclonal diversification. Blood 109, 1559-1567.
Wardemann, H., Yurasov, S., Schaefer, A., Young, J. W., Meffre, E., Nussenzweig, M. C., 2003. Predominant autoantibody production by early human B cell precursors. Science 301, 1374-1377.
Wei, X., Decker, J. M., Wang, S., Hui, H., Kappes, J. C., Wu, X., Salazar-Gonzalez, J. F., Salazar, M. G., Kilby, J. M., Saag, M. S., Komarova, N. L., Nowak, M. A., Hahn, B. H., Kwong, P. D., Shaw, G. M., 2003. Antibody neutralization and escape by HIV-1. Nature 422, 307-312.
Weller, S., Braun, M. C., Tan, B. K., Rosenwald, A., Cordier, C., Conley, M. E., Plebani, A., Kumararatne, D. S., Bonnet, D., Tournilhac, 0., Tchernia, G., Steiniger, B., Staudt, L. M., Casanova, J. L., Reynaud, C. A., Weill, J. C., 2004. Human blood IgM “memory” B cells are circulating splenic marginal zone B cells harboring a prediversified immunoglobulin repertoire. Blood 104, 3647-3654.
Weller, S., Reynaud, C. A., Weill, J. C., 2005. Splenic marginal zone B cells in humans: where do they mutate their Ig receptor? Eur. J. Immunol. 35, 2789-2792.
Xiao, X., Feng, Y., Vu, B. K., Ishima, R., Dimitrov, D. S., A large library based on a novel (CH2) scaffold: identification of HIV-1 inhibitors Biochem. Biophys. Res Commun. 387, (2009) 387-392.
Xiao, X et al., Germline-like predecessors of broadly neutralizing antibodies lack measurable binding to HIV-1 envelope glycoproteins: Implications for evasion of immune responses and design of vaccine immunogens, Biochem. Biophy. Res. Commun. (2009). Article in press.
Zhang, M. Y., Choudhry, V., Sidorov, I. A., Tenev, V., Vu, B. K., Choudhary, A., Lu, H., Stiegler, G. M., Katinger, H. W., Jiang, S., Broder, C. C., Dimitrov, D. S., 2006. Selection of a novel gp41-specific HIV-1 neutralizing human antibody by competitive antigen panning J. Immunol. Methods 317, 21-30.
Zhang, M. Y., Dimitrov, D. S., 2007. Novel approaches for identification of broadly cross-reactive HIV-1 neutralizing human monoclonal antibodies and improvement of their potency. Curr. Pharm. Des 13, 203-212.
Zhang, M. Y., Shu, Y., Phogat, S., Xiao, X., Cham, F., Bouma, P., Choudhary, A., Feng, Y. R., Sanz, I., Rybak, S., Broder, C. C., Quinnan, G. V., Evans, T., Dimitrov, D. S., 2003. Broadly cross-reactive HIV neutralizing human monoclonal antibody Fab selected by sequential antigen panning of a phage display library. J. Immunol. Methods 283, 17-25.
Zhang, M. Y., Shu, Y., Rudolph, D., Prabakaran, P., Labrijn, A. F., Zwick, M. B., Lal, R. B., Dimitrov, D. S., 2004a. Improved breadth and potency of an HIV-1-neutralizing human single-chain antibody by random mutagenesis and sequential antigen panning. J. Mol. Biol. 335, 209-219.
Zhang, M. Y., Shu, Y., Sidorov, I., Dimitrov, D. S., 2004b. Identification of a novel CD4i human monoclonal antibody Fab that neutralizes HIV-1 primary isolates from different clades. Antiviral Res. 61, 161-164.
Zhang, M. Y., Xiao, X., Sidorov, I. A., Choudhry, V., Cham, F., Zhang, P. F., Bouma, P., Zwick, M., Choudhary, A., Montefiori, D. C., Broder, C. C., Burton, D. R., Quinnan, G. V., Jr., Dimitrov, D. S., 2004c. Identification and characterization of a new cross-reactive human immunodeficiency virus type 1-neutralizing human monoclonal antibody. J. Virol. 78, 9233-9242.
Zhang, M. Y., Vu, B. K., Choudhary, A., Lu, H., Humbert, M., Ong, H., Alam, M., Ruprecht, R. M., Quinnan, G., Jiang, S., Montefiori, D. C., Mascola, J. R., Broder, C. C. Haynes, B. F., Dimitrov, D. S., Cross-reactive human immunodeficiency virus type 1-neutralizing human monoclonal antibody which recognizes a novel conformational epitope on gp41 and lacks reactivity against self antigens, J. Virol. (2008).
Zhu, Z., Bossart, K. N., Bishop, K. A., Crameri, G., Dimitrov, A. S., J. A. McEachern, J. A., Feng, Y., Middleton, D., Wang, L. F., Broder, C. C., Dimitrov, D. S., Exceptionally Potent Cross-Reactive Neutralization of Nipah and Hendra Viruses by a Human Monoclonal Antibody J. Infect. Dis. 197, (2008) 846-853.
Zhu, Z., Chakraborti, S., He, Y., Roberts, A., Sheahan, T., Xiao, X., Hensley, L. E., Prabakaran, P., Rockx, B., Sidorov, I. A., Corti, D., Vogel, L., Feng, Y., Kim, J. O., Wang, L. F., Baric, R., Lanzavecchia, A., Curtis, K. M., Nabel, G. J., Subbarao, K., Jiang, S., Dimitrov, D. S., 2007. Potent cross-reactive neutralization of SARS coronavirus isolates by human monoclonal antibodies. Proc. Natl. Acad. Sci. U.S.A.
Zhu, Z., Dimitrov, A. S., Bossart, K. N., Crameri, G., Bishop, K. A., Choudhry, V., Mungall, B. A., Feng, Y. R., Choudhary, A., Zhang, M. Y., Feng, Y., Wang, L. F., Xiao, X., Eaton, B. T., Broder, C. C., Dimitrov, D. S., 2006a. Potent neutralization of Hendra and Nipah viruses by human monoclonal antibodies. J. Virol. 80, 891-899.
Zhu, Z., Dimitrov, A. S., Chakraborti, S., Dimitrova, D., Xiao, X., Broder, C. C., Dimitrov, D. S., 2006b. Development of human monoclonal antibodies against diseases caused by emerging and biodefense-related viruses. Expert. Rev. Anti. Infect. Ther. 4, 57-66.
Zolla-Pazner, S., 2004. Identifying epitopes of HIV-1 that induce protective antibodies. Nat. Rev. Immunol. 4, 199210.
Zwick, M. B., Labrijn, A. F., Wang, M., Spenlehauer, C., Saphire, E. O., Binley, J. M., Moore, J. P., Stiegler, G., Katinger, H., Burton, D. R., Parren, P. W., 2001. Broadly neutralizing antibodies targeted to the membrane-proximal external region of human immunodeficiency virus type 1 glycoprotein gp41. J. Virol. 75, 10892-10905.
Zwick, M. B., Parren, P. W., Saphire, E. O., Church, S., Wang, M., Scott, J. K., Dawson, P. E., Wilson, I. A., Burton, D. R., Molecular features of the broadly neutralizing immunoglobulin G1b12 required for recognition of human immunodeficiency virus type 1 gp120 J. Virol. 77, (2003) 5863-5876.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A vaccine effective to elicit a desired antibody against a target antigen comprising a primary immunogen and a secondary immunogen, wherein the primary immunogen is effective to elicit B cell receptors (BCRs) that are on the maturational pathway of the desired antibody and have an intermediate degree of somatic mutational diversity, and the secondary immunogen comprises an epitope of the desired target antibody and is effective to further diversify the BCRs sufficient to form mature BCRs having the identical or substantially identical sequence as the desired antibody.

2. The vaccine of claim 1, wherein the desired target antigen is an HIV antigen.

3. The vaccine of claim 1, wherein the desired antibody is a known HIV-specific bcrnAb.

4. The vaccine of claim 3, wherein the known HIV-specific bcrnAb is b12, 2F5, 4E10, 2G12, m14, m18, m43, m44, m45, m46, m47 or m48.

5. The vaccine of claim 1, wherein the desired target antigen is a cancer antigen.

6. The vaccine of claim 1, wherein the BCRs with an intermediate degree of somatic mutational diversification have between 1% and 5% mutations relative to the corresponding germline immunoglobulin amino acid sequence.

7. The vaccine of claim 1, wherein the BCRs with an intermediate degree of somatic mutational diversification have between 5% and 10% mutations relative to the corresponding germline immunoglobulin sequence.

8. The vaccine of claim 1, wherein the BCRs with an intermediate degree of somatic mutational diversification have between 10% and 50% mutations relative to the corresponding germline immunoglobulin sequence.

9. The vaccine of claim 1, wherein the amino acid sequences of the mature BCRs are at least 90% identical to the amino acid sequence of the desired bcrnAb.

10. The vaccine of claim 1, wherein the secondary immunogen is an HIV-derived immunogen.

11. The vaccine of claim 10, wherein the HIV-specific immunogen is Env, gp160, gp140, gp120, gp41 or fragments thereof.

12. The vaccine of claim 1, wherein the secondary immunogen is a cancer-specific immunogen.

13. A method for eliciting an antibody against a desired target antigen comprising co-administering a primary immunogen and a secondary immunogen, wherein the primary immunogen is effective to elicit B cell receptors (BCRs) that are on the maturational pathway of the desired antibody and have an intermediate degree of somatic mutational diversity, and the secondary immunogen contains an epitope of the desired antibody and is effective to further diversify the BCRs to form mature BCRs having the identical or substantially identical sequence as the desired antibody.

14. The method of claim 13, wherein the desired target antigen is an HIV antigen.

15. The method of claim 13, wherein the bcrnAb is a known HIV-specific bcrnAb.

16. The method of claim 13, wherein the known HIV-specific bcrnAb is b12, 2F5, 4E10, 2G12, m14, m18, m43, m44, m45, m46, m47 or m48.

17. The method of claim 13, wherein the desired target antigen is a cancer antigen.

18. The method of claim 13, wherein the BCRs with an intermediate degree of somatic mutational diversification have between 1% and 5% mutations relative to the corresponding germline immunoglobulin amino acid sequence.

19. The method of claim 13, wherein the BCRs with an intermediate degree of somatic mutational diversification have between 5% and 10% mutations relative to the corresponding germline immunoglobulin sequence.

20. The method of claim 13, wherein the BCRs with an intermediate degree of somatic mutational diversification have between 10% and 50% mutations relative to the corresponding germline immunoglobulin sequence.

21. The method of claim 13, wherein the amino acid sequences of the mature BCRs are at least 90% identical to the amino acid sequence of the desired bcrnAb.

22. The method of claim 13, wherein the secondary immunogen is an HIV-specific immunogen.

23. The method of claim 22, wherein the HIV-derived immunogen is Env, gp160, gp140, gp120, gp41 or fragments thereof.

24. The method of claim 13, wherein the secondary immunogen is a cancer-related immunogen.

25. A method for vaccinating a subject against a disease comprising a target antigen, the method comprising co-administering a primary immunogen and a secondary immunogen, wherein the primary immunogen is effective to elicit B cell receptors (BCRs) that are on the maturational pathway of a desired antibody specific for the target antigen and which have an intermediate degree of somatic mutational diversity, and the secondary immunogen contains an epitope of the desired antibody and is effective to further diversify the BCRs to form mature BCRs having the identical or substantially identical sequence as the desired antibody.

26. The method of claim 25, wherein the disease is HIV and the desired target antigen is an HIV antigen.

27. The method of claim 25, wherein the antibody is a known HIV-specific bcrnAb.

28. The method of claim 27, wherein the known HIV-specific bcrnAb is b12, 2F5, 4E10, 2G12, m14, m18, m43, m44, m45, m46, m47 or m48.

29. The method of claim 25, wherein the disease is cancer and the desired target antigen is a cancer or cancer-related antigen.

30. The method of claim 25, wherein the BCRs with an intermediate degree of somatic mutational diversification have between 1% and 5% mutations relative to the corresponding germline immunoglobulin amino acid sequence.

31. The method of claim 25, wherein the BCRs with an intermediate degree of somatic mutational diversification have between 5% and 10% mutations relative to the corresponding germline immunoglobulin sequence.

32. The method of claim 25, wherein the BCRs with an intermediate degree of somatic mutational diversification have between 10% and 50% mutations relative to the corresponding germline immunoglobulin sequence.

33. The method of claim 25, wherein the amino acid sequences of the mature BCRs are at least 90% identical to the amino acid sequence of the desired bcrnAb.

34. The method of claim 25, wherein the secondary immunogen is an HIV-derived immunogen.

35. The method of claim 34, wherein the HIV-derived immunogen is Env, gp160, gp140, gp120, gp41 or fragments thereof.

36. The method of claim 25, wherein the secondary immunogen is a cancer-related immunogen.
